(19) United States
(12) Patent Application Publication
(10) Pub. No.: US 2024/0018141 A1
(43) Pub. Date: Jan. 18, 2024

(54) IMMUNOMODULATORS AND IMMUNOMODULATOR CONJUGATES

(71) Applicant: Regents of the University of Minnesota, Minneapolis, MN (US)

(72) Inventors: David M. FERGUSON, Minneapolis, MN (US); Peter Gustav LARSON, Saint Paul, MN (US)

(21) Appl. No.: 18/236,004

(22) Filed: Aug. 21, 2023

Related U.S. Application Data
(63) Continuation of application No. PCT/US2022/017348, filed on Feb. 22, 2022.
(60) Provisional application No. 63/152,003, filed on Feb. 22, 2021.
(60) Provisional application No. 63/273,081, filed on Oct. 28, 2021.

(51) Int. Cl. C07D 471/04; C07D 513/04; C07D 491/048; A61P 37/04

(52) U.S. Cl. CPC C07D 471/04; C07D 513/04; C07D 491/048; A61P 37/04

(57) ABSTRACT

The present disclosure provides compounds of Formula I: (I), pharmaceutically acceptable salts thereof, conjugates thereof, as well as methods of synthesizing and using such compounds, e.g., in a method of treating a pathological condition in a subject. The compounds described herein can have immunomodulatory properties and can interact with a Toll-like receptor as described herein.

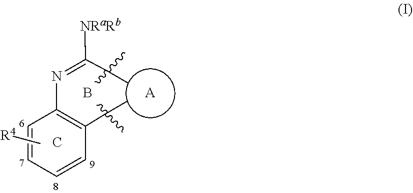

### CROSS REFERENCE TO RELATED APPLICATIONS

This application claims priority from U.S. Provisional Application No. 63/152,003, filed on Feb. 22, 2021, and U.S. Provisional Application No. 63/273,081, filed on Oct. 28, 2021, which are hereby incorporated by reference in their entireties.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE DISCLOSURE

Toll-like receptor (TLR) modulators can be used in various therapeutic settings, e.g., as vaccine adjuvants. However, many factors have limited the efficacy of TLR modulating compounds such as imiquimod. As an example, although such compounds can induce proinflammatory cytokines, they may also concurrently induce significant levels of anti-inflammatory cytokines such as IL-10. Thus, there exists an unmet need to develop TLR modulating compounds that can trigger a more desirable ratio of pro- to anti-inflammatory cytokines.

### SUMMARY OF THE DISCLOSURE

In some embodiments, TLR modulating compounds that can trigger a more desirable ratio of pro- to anti-inflammatory cytokines are disclosed. In some instances, such TLR modulating compounds can be a compound of Formula I:

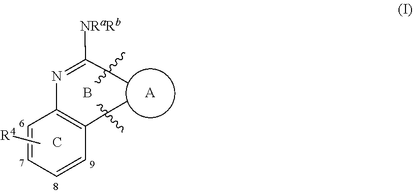

wherein:
the fused ring A is selected from the group consisting of:

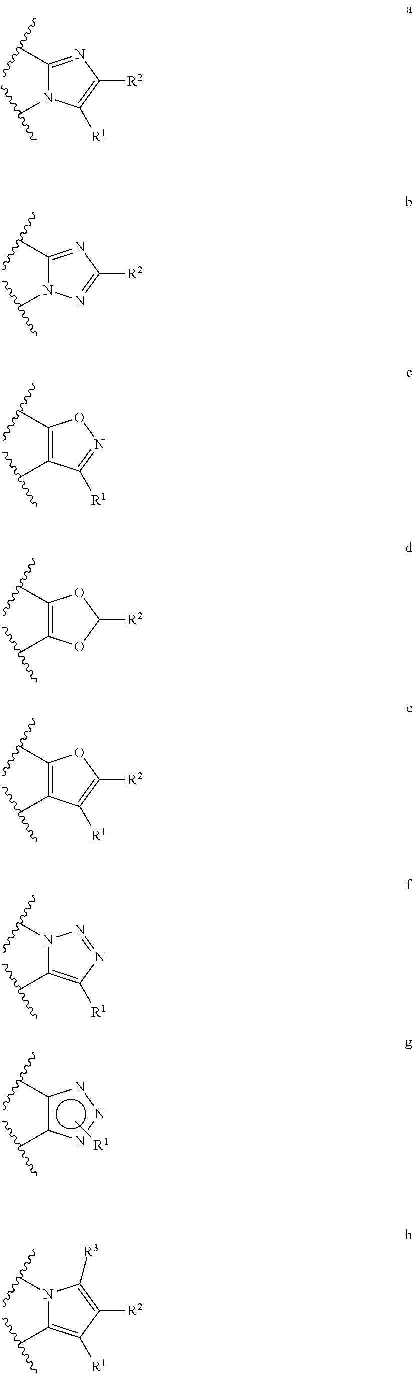

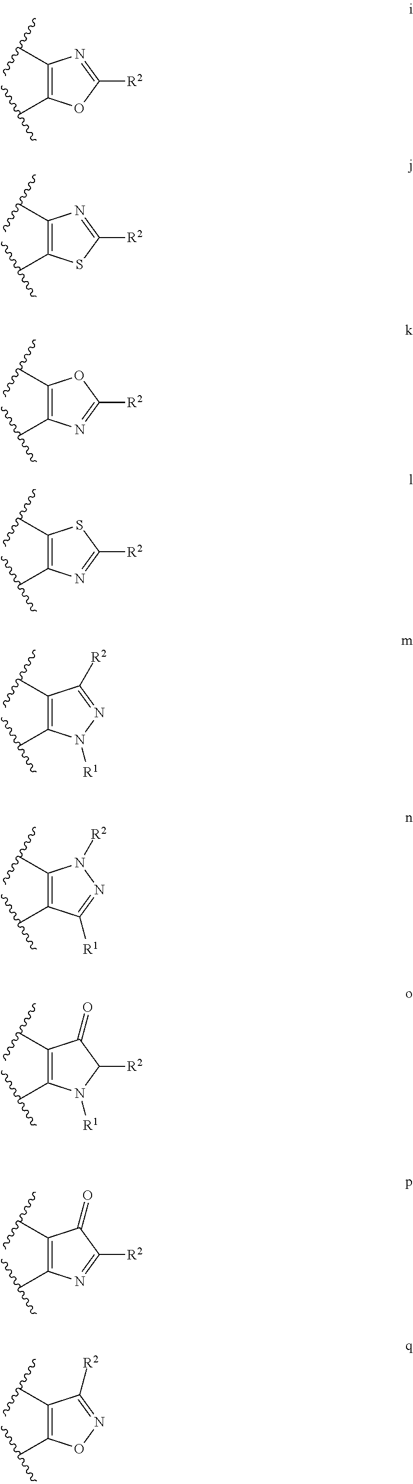

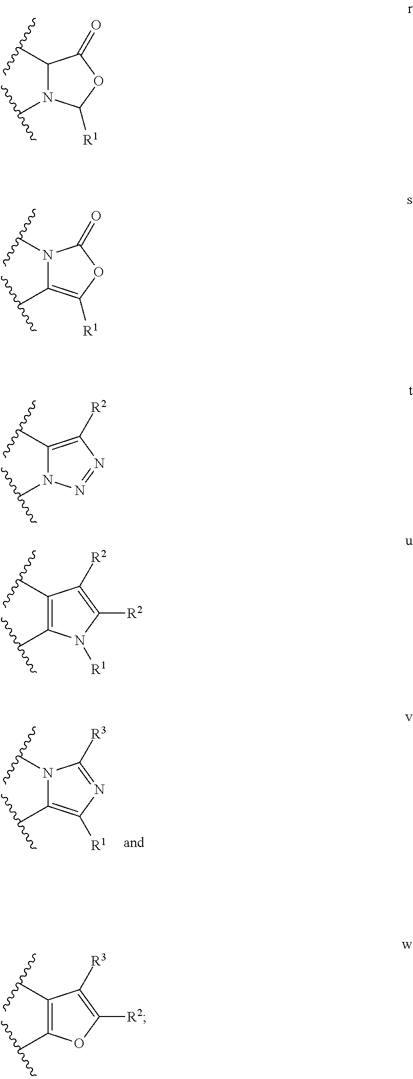

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in Formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some embodiments, Y is an antigen.

The present disclosure also provides a pharmaceutical composition comprising a compound of Formula I, or a pharmaceutically acceptable salt thereof, and a pharmaceutically acceptable diluent and/or carrier.

Further provided herein is a method for treating a pathological condition (e.g. a viral infection, a bacterial infection, or a cancer) in an animal (e.g., a mammal such as a human) comprising administering to the animal a compound of Formula I, or a pharmaceutically acceptable salt thereof, and/or a pharmaceutical composition comprising such compound.

Further provided herein is a method for stimulating an immune response in an animal (e.g., a mammal such as a human), the method comprising administering to the animal a compound of Formula I, or a pharmaceutically acceptable salt thereof, and/or a pharmaceutical composition comprising such compound.

Further provided herein is a compound of Formula I, or a pharmaceutically acceptable salt thereof, for use in the prophylactic and/or therapeutic treatment of a pathological condition (e.g. a viral infection, a bacterial infection, or a cancer) in an animal (e.g., a mammal such as a human). Further provided herein is a compound of Formula I, or a pharmaceutically acceptable salt thereof, for use in medical therapy. The present disclosure also provides the use of a compound of Formula I, or a pharmaceutically acceptable salt thereof, for the manufacture of a medicament useful for the treatment of a pathological condition (e.g. a viral infection, a bacterial infection, or a cancer) in an animal (e.g., a mammal such as a human).

The disclosure further provides processes and intermediates disclosed herein that are useful for preparing compounds of Formula I-IV, or pharmaceutically acceptable salts thereof.

Further provided herein are methods for synthesizing compounds of Formulae I-IV and intermediates thereof, the methods comprising performing a reaction according to any one of reaction SCHEMES 1-5.

### DETAILED DESCRIPTION

I. Introduction

The present disclosure provides compounds (e.g., those of Formulae I-IV) capable of interacting with one or more Toll-like receptors (TLRs), e.g., TLR-7, TLR-8, or a combination thereof. In some embodiments, the compounds of the present disclosure are agonistic to one or more TLRs. Thus, in some embodiments, the compounds provided herein can initiate an immune response in a subject, which can be used to treat a disease or pathological condition in the subject. Further provided herein are conjugates that can comprise a compound of the present disclosure (e.g., one of Formulae I-IV) coupled to another molecule. Such other molecule can be a small molecule, a peptide, a protein, or a nucleic acid. In some cases, a conjugate herein comprises a compound of any one of Formulae I-IV coupled to a peptide or protein. The compound can be coupled (e.g., covalently coupled) to the peptide or protein via a linker, as further described herein.

Number ranges are to be understood as inclusive, i.e., including the indicated lower and upper limits. Furthermore, the term “about”, as used herein, and unless clearly indicated otherwise, generally refers to and encompasses plus or minus 10% of the indicated numerical value(s). For example, “about 10%” may indicate a range of 9% to 11%, and “about 1” may include the range 0.9-1.1.

It is noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

The term “subject,” as used herein, generally refers to an individual to which a compound as described herein, or conjugate comprising such compound, is administered. Examples of a “subject” include, but are not limited to, a mammal such as a human, rat, mouse, guinea pig, non-human primate, pig, goat, cow, horse, dog, cat, bird, and fowl. Typically, a subject is a rat, mouse, dog, non-human primate, or human. In some aspects, the subject is a human.

The term “antibody,” as used herein, covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds can be disrupted, that exhibit the desired biological activity and provided that the antigen binding antibody fragments have the requisite number of attachment sites, if applicable, for a desired number of attached groups, such as a linker moiety, as described herein. In aspects which include compounds attached to an antibody via linker, such linker can be attached via a succinimide or hydrolyzed succinimide to the sulfur atoms of cysteine residues of reduced interchain disulfide bonds and/or cysteine residues of the introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. The antibody is derivable from any suitable species. In some aspects, the antibody is of human or murine origin, and in some aspects the antibody is a human, humanized or chimeric antibody. Antibodies can be fucosylated to varying extents or afucosylated.

An “antigen” is an entity to which an antibody specifically binds. Generally, an “antigen,” as used herein, includes any substance that causes the immune system of an animal to produce antibodies or antigen-specific T cells against the substance. The term also includes haptans. An antigen may be a foreign substance from the environment such as a chemical, bacteria, virus, or pollen. An antigen may also be formed within the body such as with bacterial toxins, tissue cells, or tumor cells. The antigen is the molecular structure encoded by the substance such as the pathogen or tumor against which the immune response is directed. Examples of antigens may come from pathogens such as bacteria or viruses (e.g. influenza, HIV, or HCV). Alternatively, the antigen may come from a tumor cell or a tumor cell lysate or synthetic peptides derived from tumors or infectious organisms. In one embodiment, the antigen comprises a peptide sequence containing cysteine or lysine.

All terms, chemical names, expressions, and designations have their usual meanings which are well-known to those skilled in the art. When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

The term “optionally substituted,” refers to an indicated group being either substituted or unsubstituted. As used herein, the term “substituted” refers to a compound (e.g., an alkyl chain) wherein a hydrogen is replaced by another reactive functional group or atom, as described herein.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, a broken line in a chemical structure can be used to indicate a bond to the rest of the molecule. For example, [symbol] in

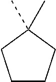

is used to designate the 1-position as the point of attachment of 1-methylcyclopentate to the rest of the molecule. Alternatively,

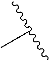

in, e.g.,

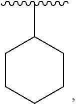 [SMILES: *C1CCCCC1]

can be used to indicate that the given moiety, the cyclohexyl moiety in this example, is attached to a molecule via the bond that is “capped” with the wavy line.

The following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane.

Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoro-methylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “acyl” refers to an alkyl, haloalkyl, alkenyl, alkynyl, aryl cycloalkyl, heteroaryl, or heterocyclyl group, as defined herein, connected to the remainder of the compound by a C═O (carbonyl) group.

The term “carboxamido” refers to a —C(═O)NRR′ group, wherein R and R′ are independently selected from the group consisting of hydrogen, alkyl, alkenyl, alkynyl, aryl cycloalkyl, heteroaryl, and heterocyclyl, as defined herein.

The term “heteroalkylene” refers to a bivalent unsubstituted straight or branched group derived from heteroalkyl (e.g., as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂— —C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N+(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “cycloalkyl” refers to a cyclic, saturated, or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated π system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group. The term “cyano” refers to a —CN group. The term “carboxy” refers to a —C(═O)OH group. The term “oxo” refers to a ═O group.

The term “alkanoyl” refers to an alkyl group, as defined herein, connected to the remainder of the molecule by a —C(═O) group. Exemplary alkanoyl groups include, but are not limited to acetyl, n-propanoyl, and n-butanoyl.

The term “alkanoyloxy” refers to an alkyl group, as defined herein, connected to the remainder of the molecule by an —OC(═O) group. Exemplary alkanoyloxy groups include, but are not limited to acetoxy, n-propanoyloxy, and n-butanoyloxy.

The term “alkoxycarbonyl” refers to an alkoxy group, as defined herein, connected to a C(═O)-alkyl group via the oxygen atom of the alkoxy (i.e., an alkyl ester group).

The terms “arylalkyl” and “cycloalkylalkyl” refer to an aryl group or a cycloalkyl group (as defined herein) connected to the remainder of the molecule by an alkyl group, as defined herein. Exemplary arylalkyl groups include but are not limited to benzyl and phenethyl. Exemplary cycloalkylalkyl groups include, but are not limited to cyclopropylmethyl, cyclobutylmethyl, cyclopentylethyl, and cyclohexylethyl.

As to any of the above groups that contain one or more substituents, it is understood that such groups do not contain any substitution or substitution patterns which are sterically impractical and/or synthetically non-feasible. In addition, as further described herein, the compounds of this disclosure can include all stereochemical isomers (and racemic mixtures) arising from the substitution of these compounds.

The term “amino acid,” as used herein, comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine.

The term “peptide,” as used herein, describes a sequence of at least about 2 and not more than about 25 amino acids and/or peptidyl residues. The peptide sequence may be linear or cyclic. As an example, a cyclic peptide can be prepared or may result from the formation of disulfide bridges between two cysteine residues in a sequence. A peptide can be linked to the remainder of a compound of Formula I through the carboxy terminus, the amino terminus, or through any other convenient point of attachment, such as, for example, through the sulfur of a cysteine. In some cases, a peptide herein comprises 3 to 25, 5 to 21, or 10 to 25 amino acids. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right. A “protein,” as used herein, generally refers to a molecule comprising a sequence of at least about 25 amino acids and/or peptidyl residues, such as from about 25 to about 750, from about 50 to about 500, from about 100 to about 500, or from about 100 to about 1000.

As used herein, the term “free drug” refers to a biologically active drug molecule (e.g., one of Formulae I-IV) that is not covalently attached to another moiety, such as a peptide or protein. Accordingly, free drug refers to a compound as it exists immediately upon cleavage from a conjugate, e.g., a drug-peptide or drug-protein conjugate as described herein. The release mechanism can be via a cleavable linker in the conjugate, or via intracellular conversion or metabolism of the conjugate. In some aspects, the free drug can be protonated and/or may exist as a charged moiety. The free drug is a pharmacologically active species which is capable of exerting a particular biological effect. In some embodiments, the pharmacologically active species is the parent drug alone. In some embodiments, the pharmacologically active species is the parent drug bonded to another molecule, e.g., in a conjugate.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment,” in some aspects, also includes prolonging survival of a subject as compared to expected survival if not receiving treatment.

In the context of cancer, the term “treating” includes any or all of: inhibiting growth of cancer cells or of a tumor; inhibiting replication of cancer cells, lessening of overall tumor burden or decreasing the number of cancer cells, and ameliorating one or more symptoms associated with the disease.

II. TLR-Modulating Compounds

In some embodiments, provided herein are Toll-like receptor (TLR) modulating compounds that can trigger a more desirable ratio of pro- to anti-inflammatory cytokines. In some instances, a compound of the present disclosure can interact and/or modulate a TLR-7, a TLR-8, or a combination of such receptors.

Compound Structure

In some instances, provided herein is a compound of Formula I capable of modulating a TLR:

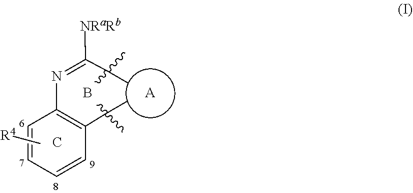

wherein:
the fused ring A is selected from the group consisting of:

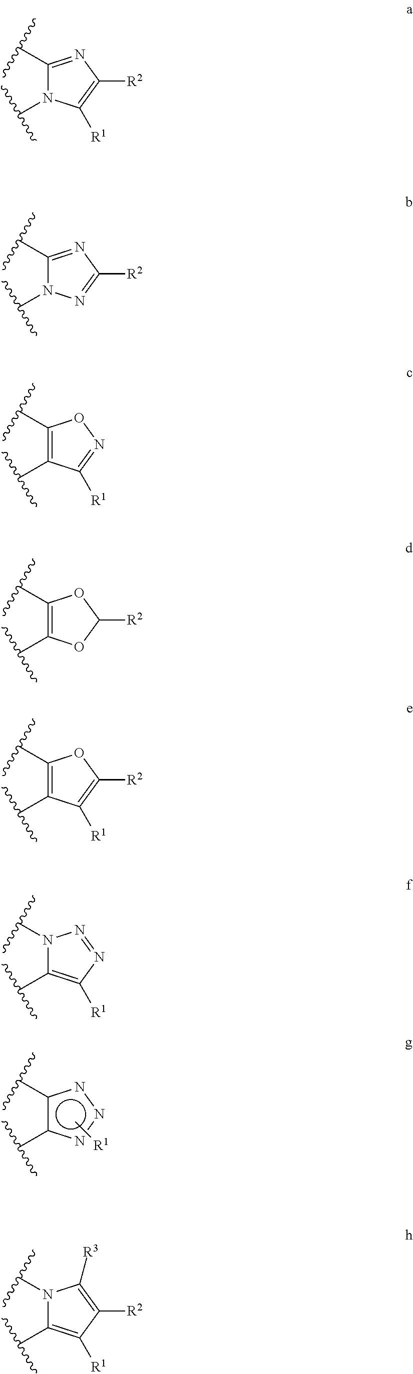

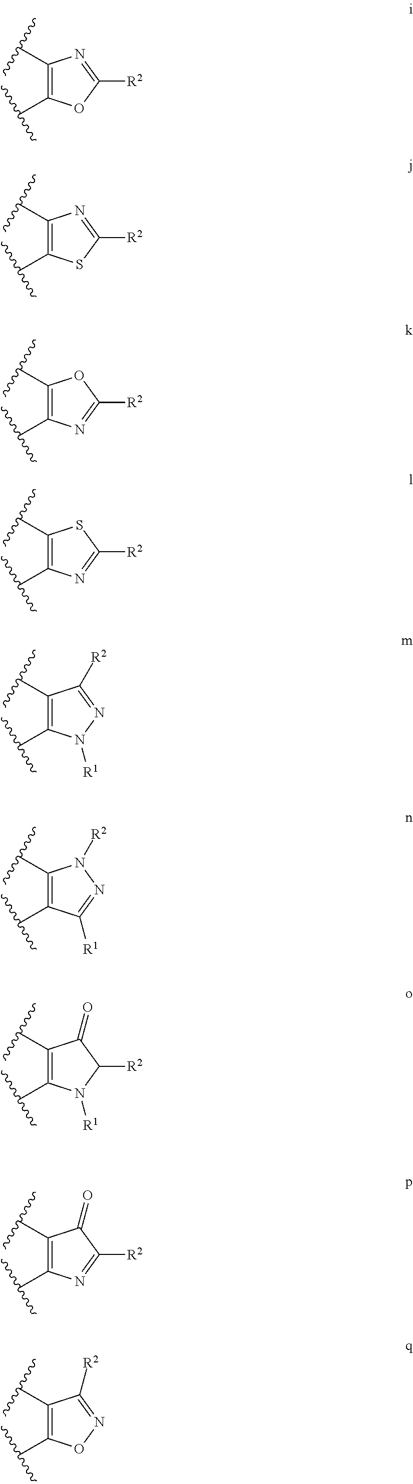

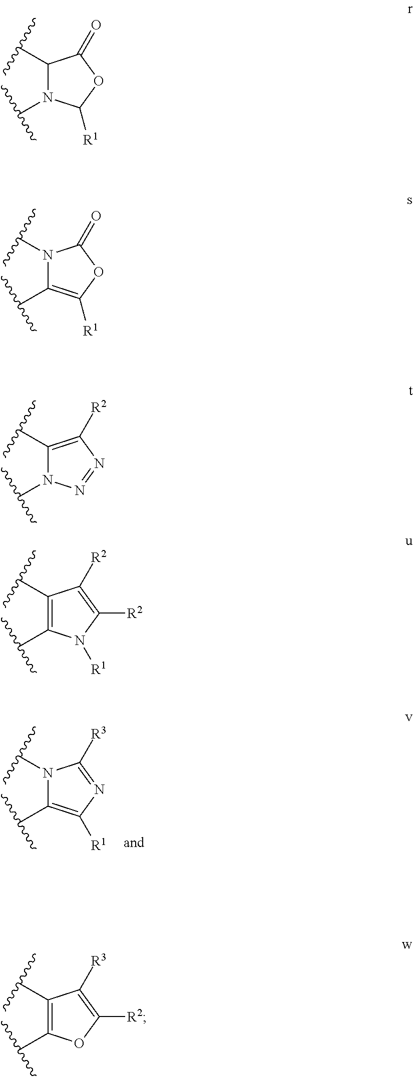

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{f}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some instances, provided herein is a compound of Formula I capable of modulating a TLR:

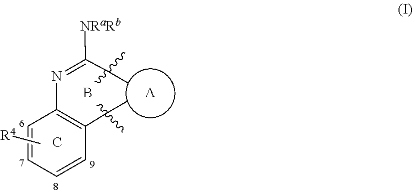

wherein:
the fused ring A is selected from the group consisting of:

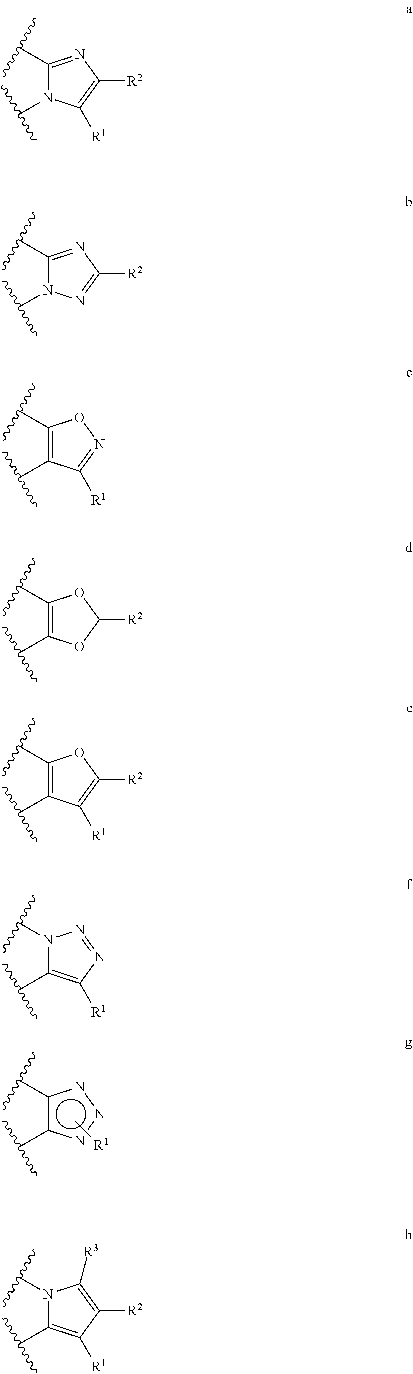

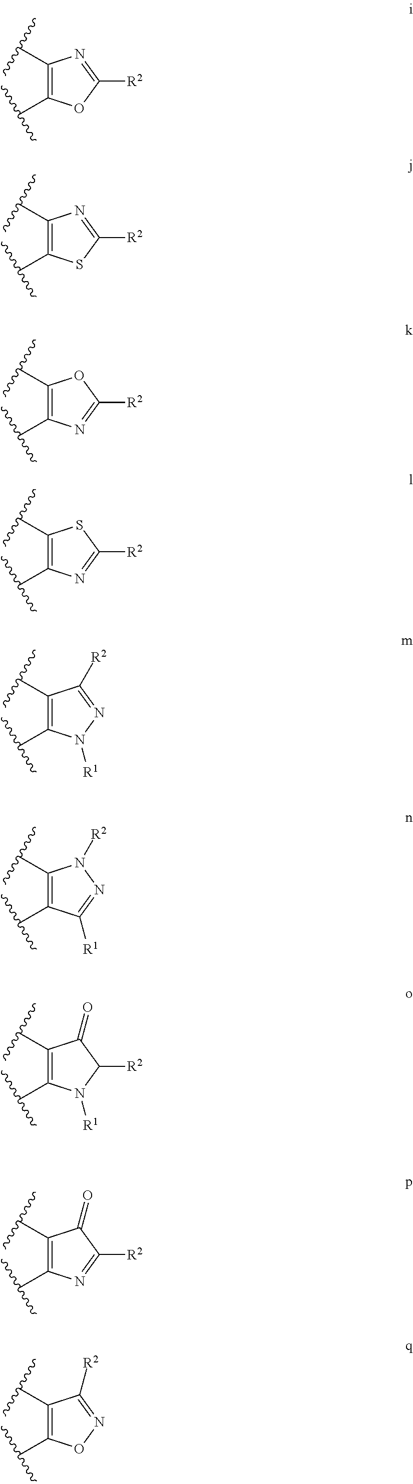

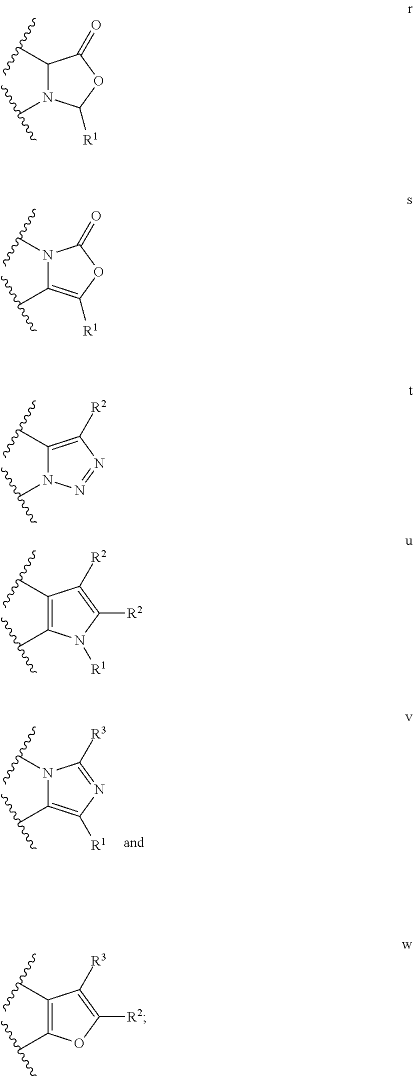

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{w}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some instances, provided herein is a compound of Formula I capable of modulating a TLR:

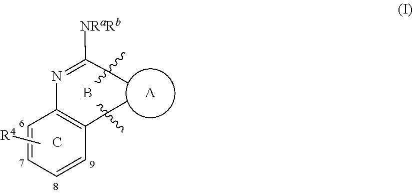

wherein:
the fused ring A is selected from the group consisting of:

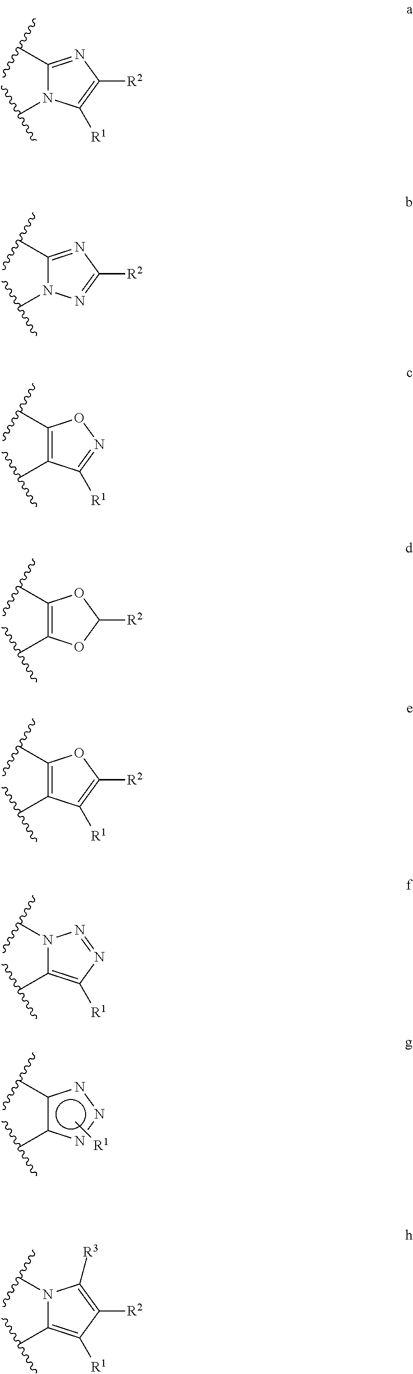

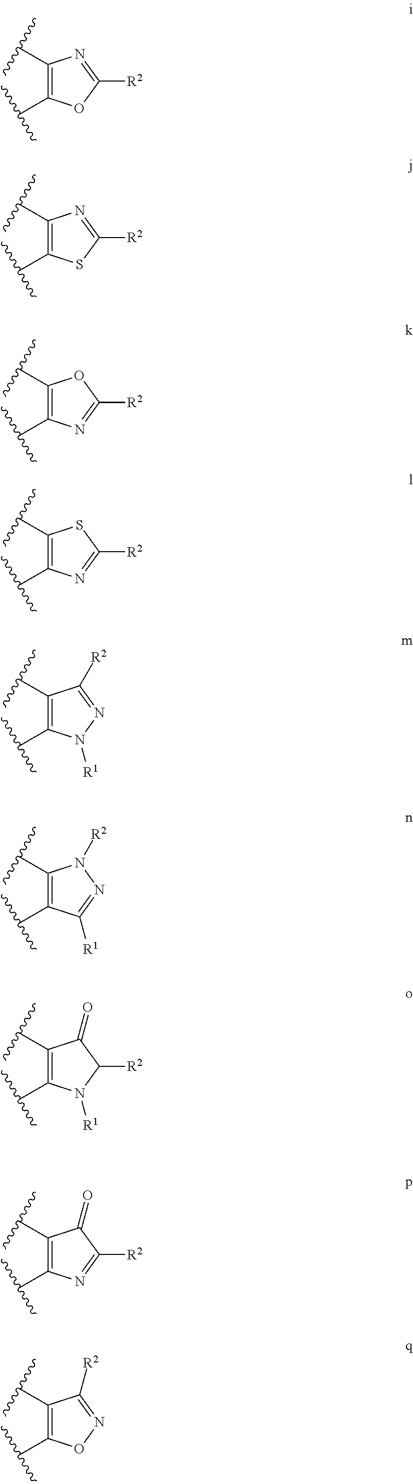

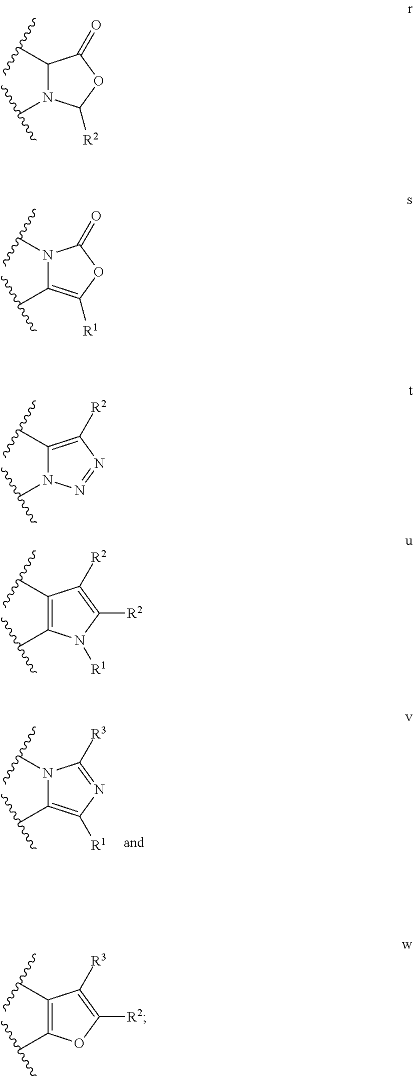

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some instances, provided herein is a compound of Formula I capable of modulating a TLR:

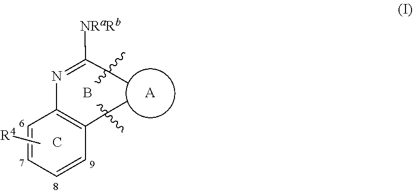

wherein:
the fused ring A is:

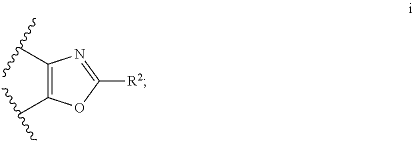

R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide

In some instances, provided herein is a compound of Formula I capable of modulating a TLR:

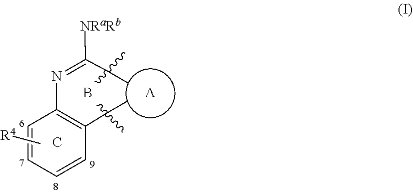

wherein:
the fused ring A is:

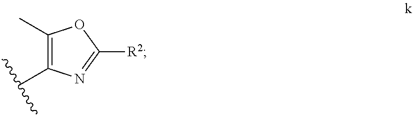

R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some instances, provided herein is a compound of Formula I capable of modulating a TLR:

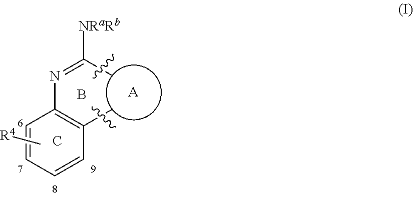

wherein:
the fused ring A is:

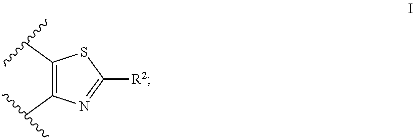

R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some instances, provided herein is a compound of Formula I capable of modulating a TLR:

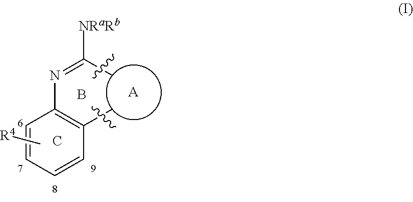

wherein:
the fused ring A is:

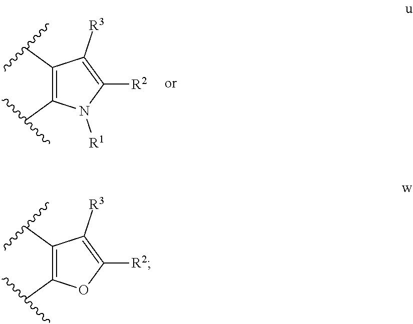

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}; and
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some instances, provided herein, a compound of Formula I comprises a structure of Formula III or IV:

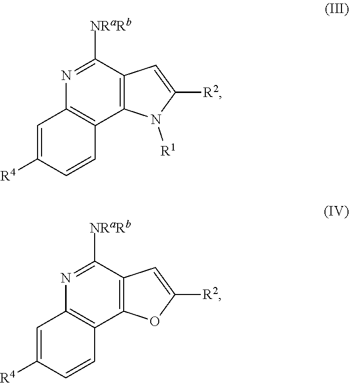

or a pharmaceutically acceptable salt thereof,
wherein:
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, or R^{k}—S(O)₂—O—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some instances, provided herein is a compound of Formula I capable of modulating a TLR:

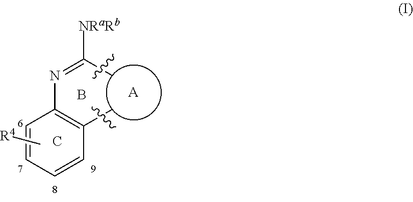

wherein:
the fused ring A is selected from the group consisting of:

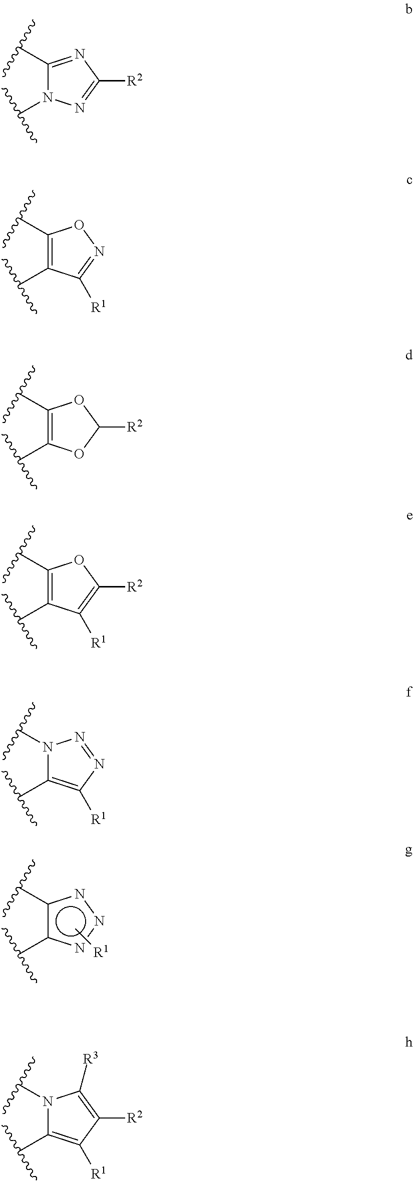

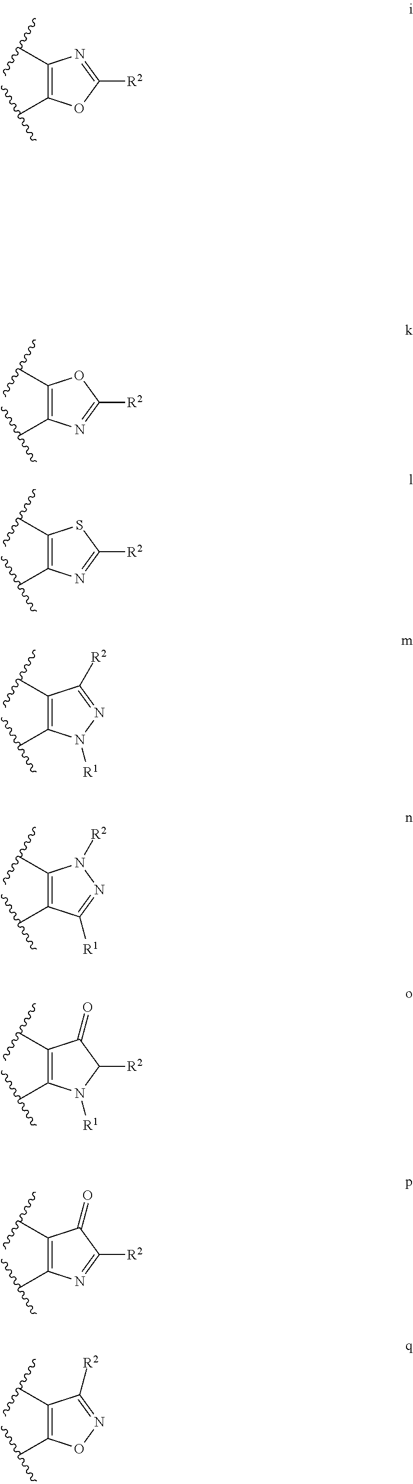

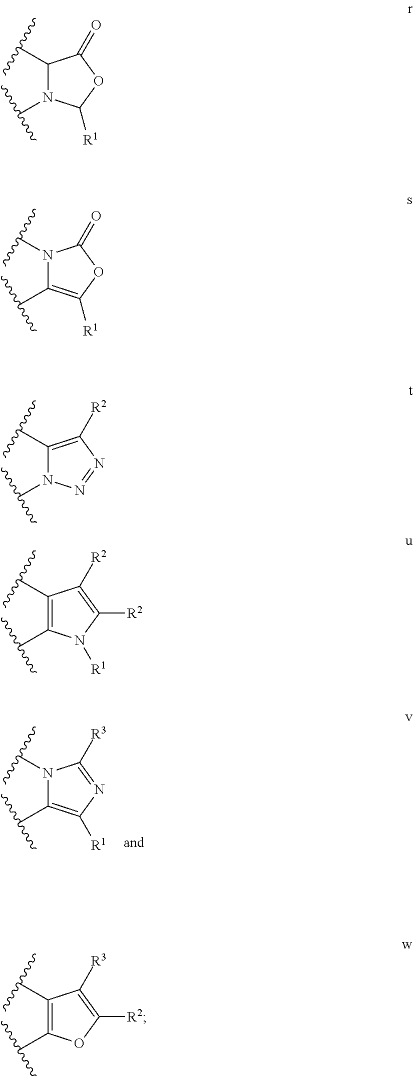

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some instances, provided herein is a compound of Formula I capable of modulating a TLR:

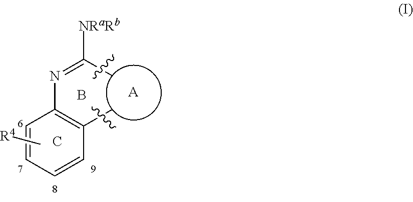

wherein:
the fused ring A is selected from the group consisting of:

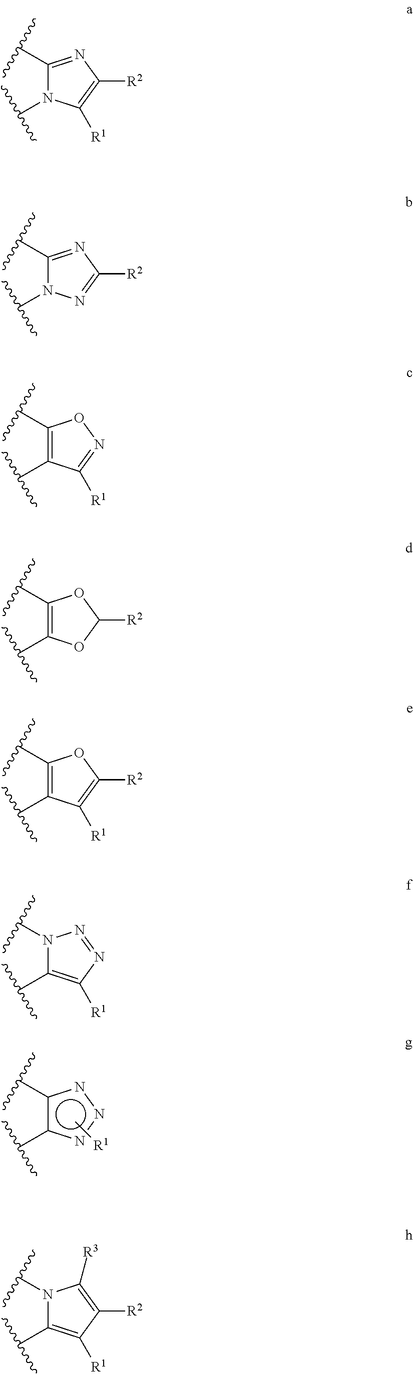

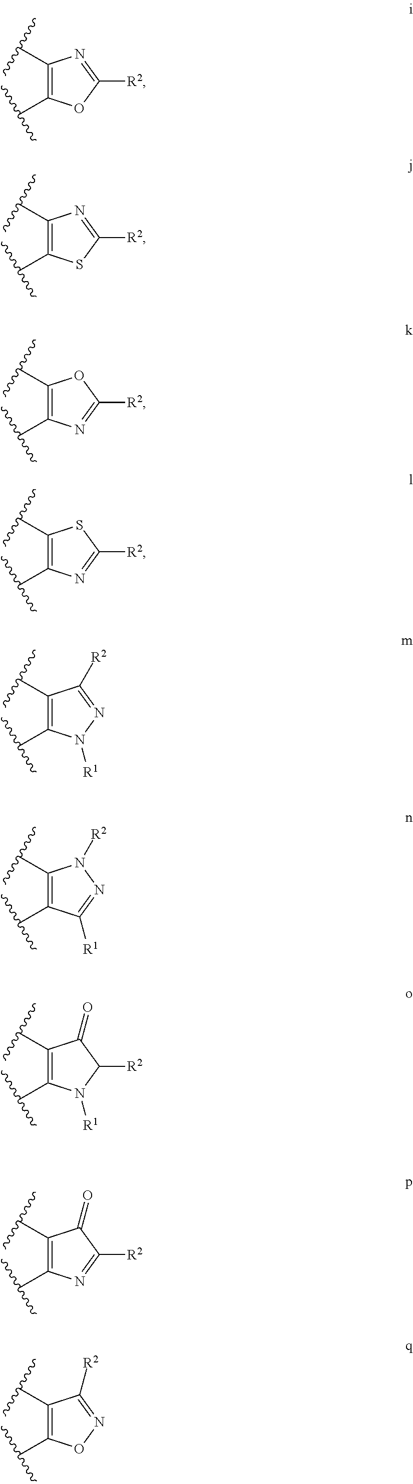

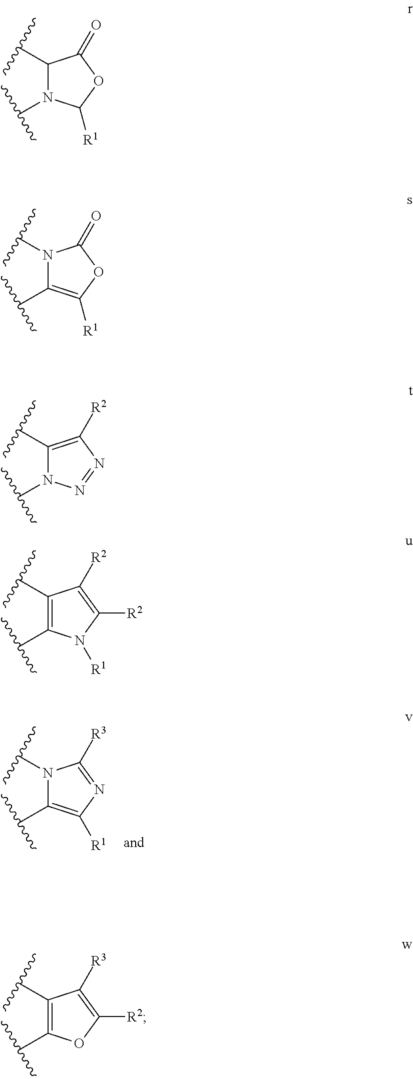

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, or (C₁-C₆)alkoxycarbonyl, wherein any (C₁-C₆)alkyl, (C₂-C₆)alkenyl, (C₂-C₆)alkynyl, (C₁-C₆)alkanoyl, and (C₁-C₆)alkoxycarbonyl, is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

In some embodiments, the compound of Formula I comprises a structure of Formula (Ia) or Formula (Ib):

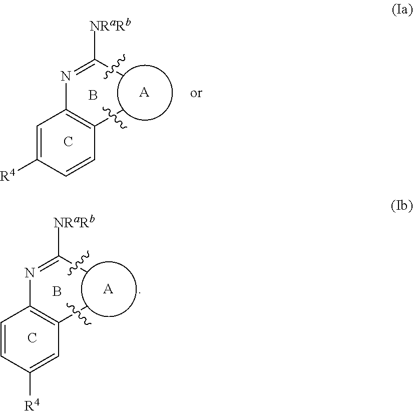

In some embodiments, the compound of Formula I comprises a structure of Formula (Ia):

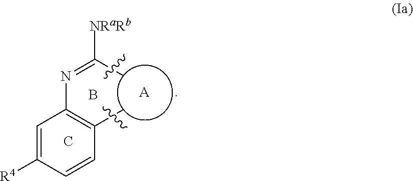

In some embodiments, the compound of Formula I comprises a structure of Formula (Ib):

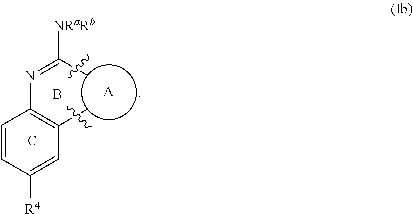

In some embodiments, the fused ring A is selected from the group consisting of:

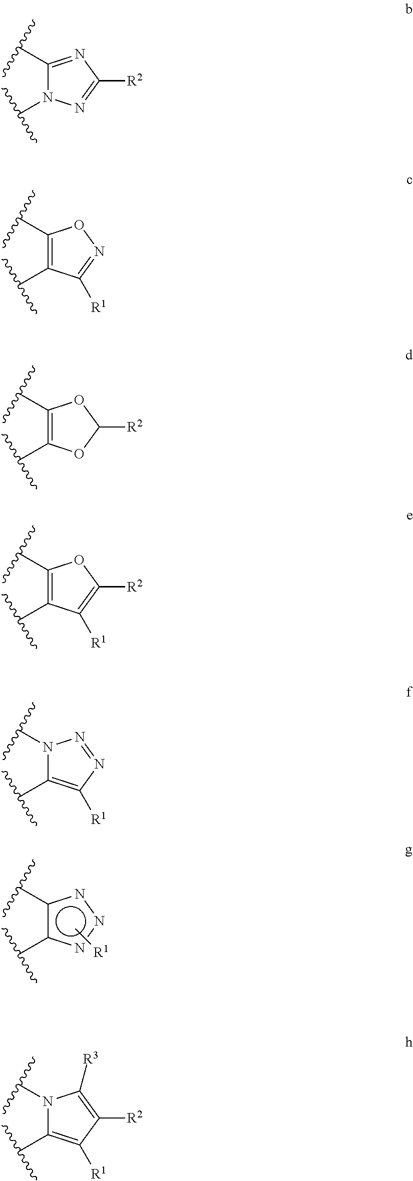

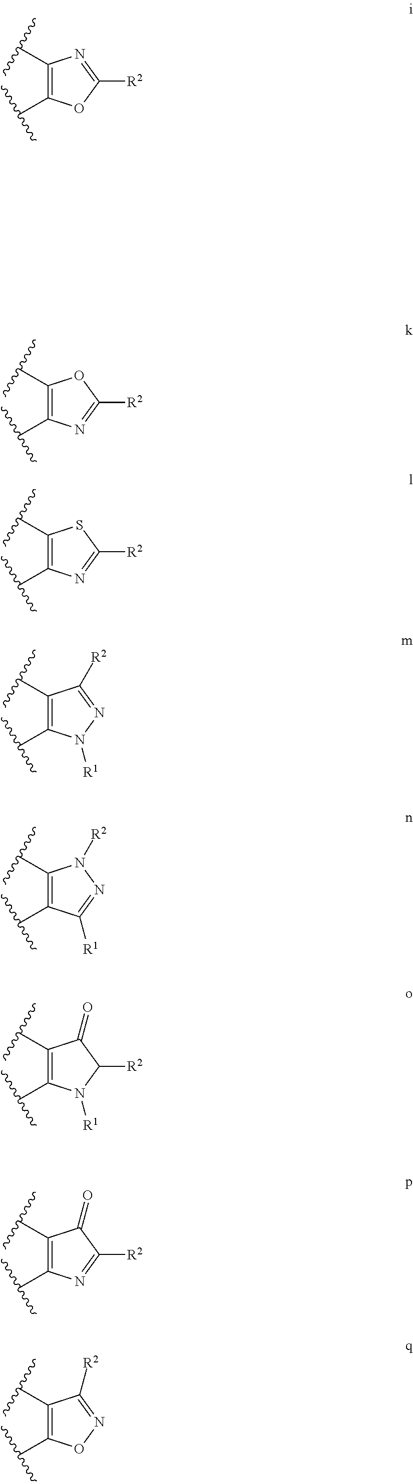

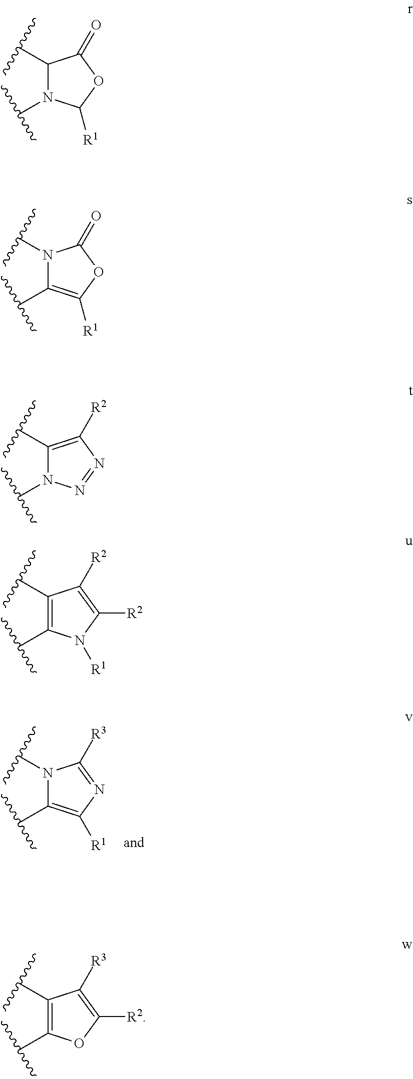

In some embodiments, the fused ring A is selected from the group consisting of:

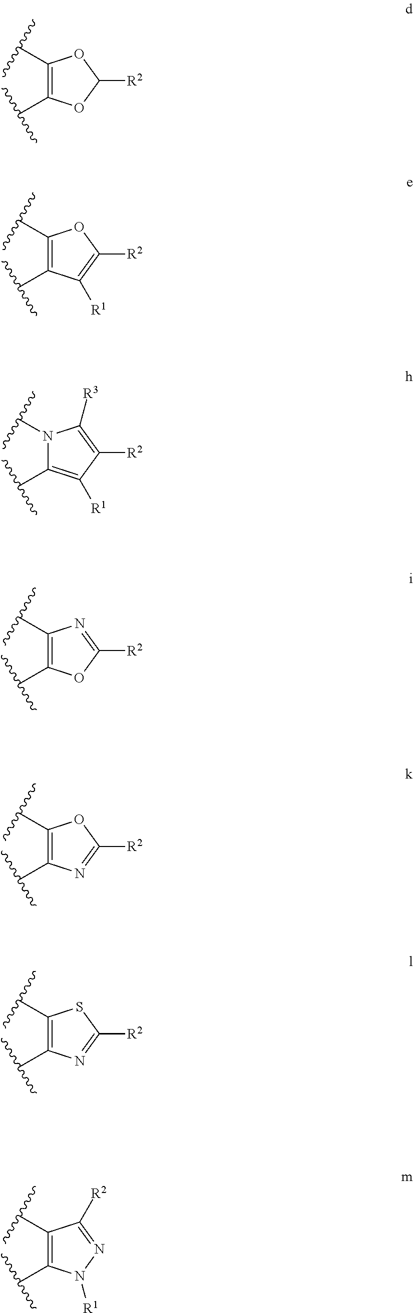

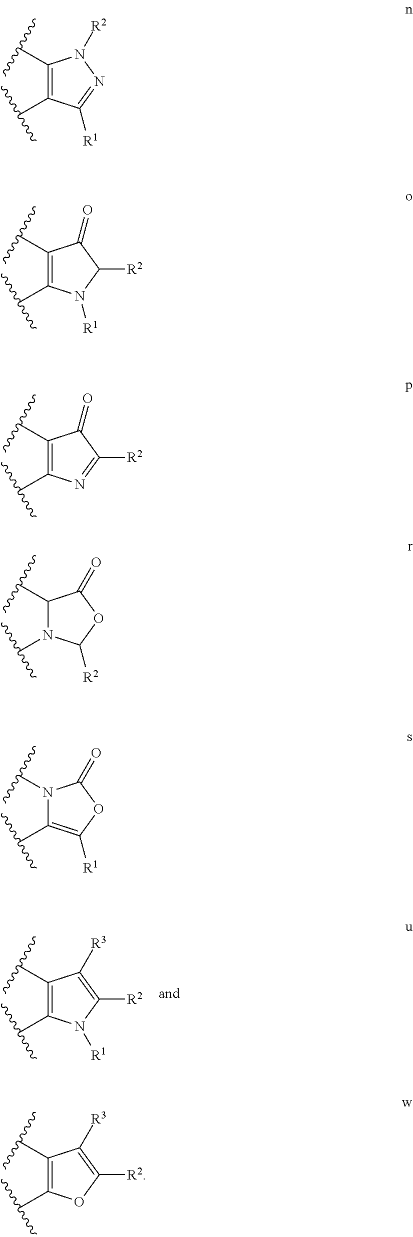

In some embodiments, the fused ring A is selected from the group consisting of:

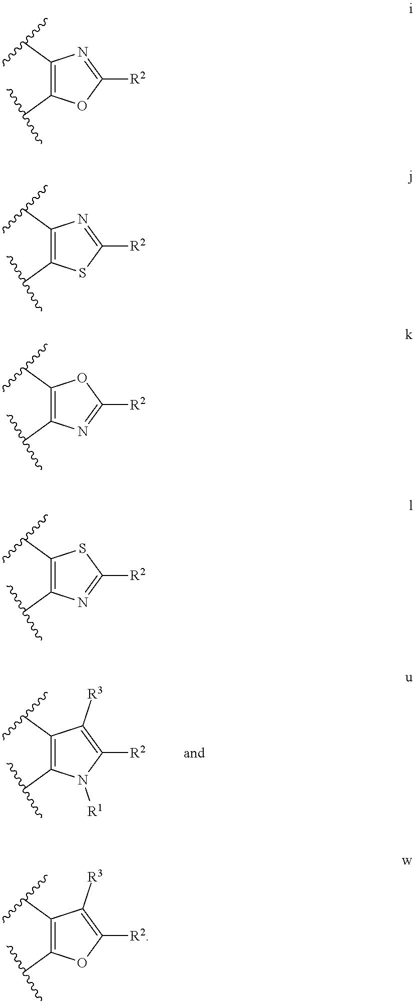

In some embodiments, the fused ring A is selected from the group consisting of:

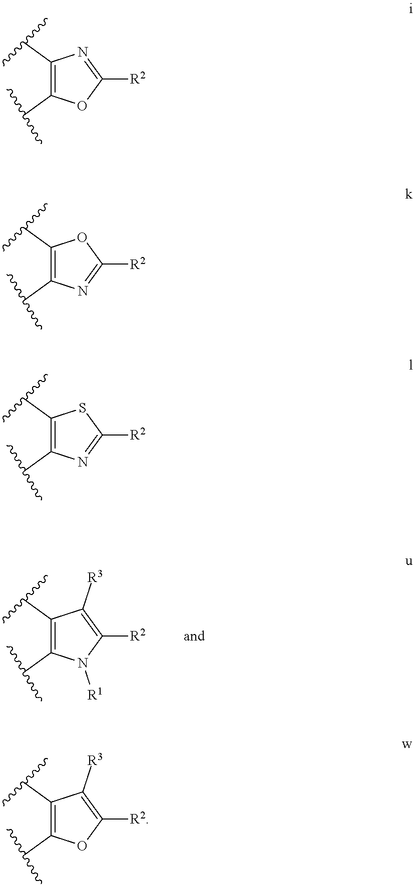

In some embodiments, the fused ring A is selected from the group consisting of:

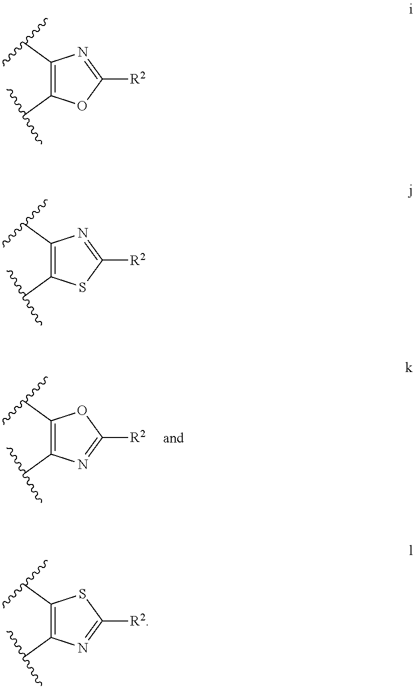

In some embodiments, the fused ring A is selected from the group consisting of:

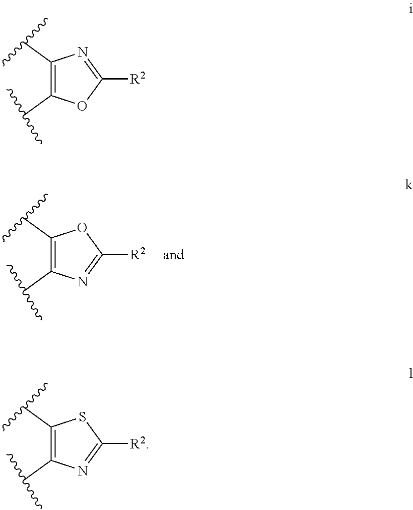

In some embodiments, the fused ring A is selected from the group consisting of:

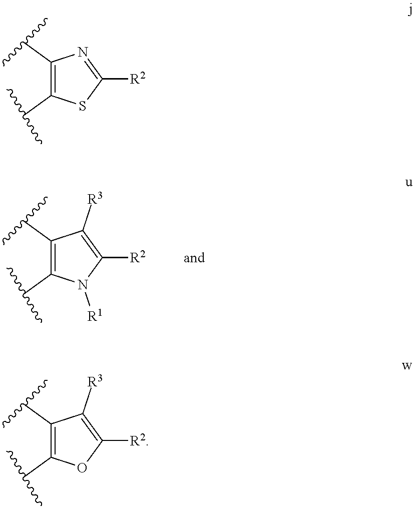

In some embodiments, the fused ring A is selected from the group consisting of:

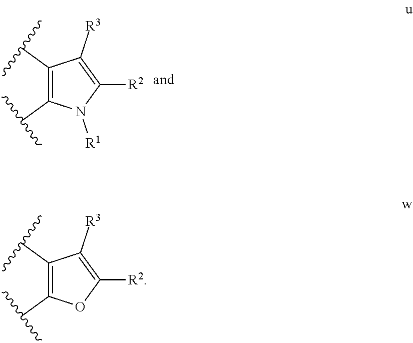

In some embodiments, the fused ring A is

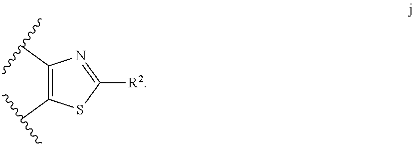

In some embodiments, the fused ring A is

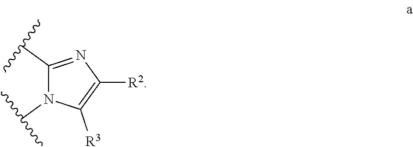

In some embodiments, the fused ring A is

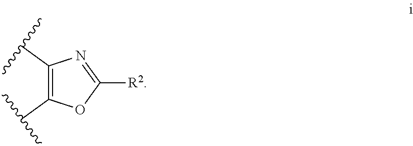

In some embodiments, the fused ring A is

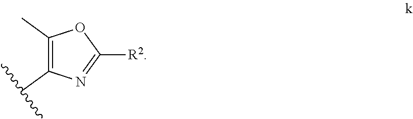

In some embodiments, the fused ring A is

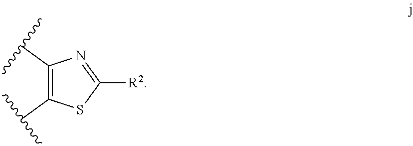

In some embodiments, the fused ring A is

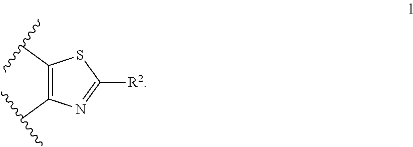

In some embodiments, R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy.

In some embodiments, R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy.

In some embodiments, R¹ is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy.

In some embodiments, R¹ is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, cyano, oxo, oxiranyl, (C₃-C₈)cycloalkyl.

In some embodiments, R¹ is H, (C₁-C₆)alkyl, (C₂-C₆)alkenyl, (C₂-C₆)alkynyl, or —NR^{g}R^{h}, wherein any (C₁-C₆)alkyl, (C₂-C₆)alkenyl, and (C₂-C₆)alkynyl is optionally substituted with one or more groups independently selected from the group consisting of halo, cyano, oxo, oxiranyl, (C₃-C₈)cycloalkyl.

In some embodiments, R¹ is H or (C₁-C₆)alkyl optionally substituted with one or more groups independently selected from the group consisting of halo and cyano. R¹ is H or (C₁-C₆)alkyl.

In some embodiments, R¹ is H. In some embodiments, R¹ is not H.

In some embodiments, R¹ is R^{c}R^{d}NS(O)₂— or R^{e}S(O)₂NR^{f}— when R² is NR^{g}R^{h}. In some cases, R¹ is R^{c}R^{d}NS(O)₂— or R^{e}S(O)₂NR^{f}— when R² is (C₁-C₆)alkyl substituted with one or more (C₁-C₆)alkylthio.

In some embodiments, R² is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

In some embodiments, R² is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, —SH, cyano, (C₃-C₈)cycloalkyl, and NR^{g}R^{h}.

In some embodiments, R² is H, (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of halo, —SH, cyano, (C₃-C₈)cycloalkyl, and NR^{g}R^{h}.

In some embodiments, R² is, (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of halo, —SH, cyano, (C₃-C₈)cycloalkyl, and NR^{g}R^{h}.

In some embodiments, R² is H or (C₁-C₆)alkyl optionally substituted with one or more groups independently selected from the group consisting of halo, —SH, cyano, and NR^{g}R^{h}.

In some embodiments, R² is wherein R² is H or (C₁-C₆)alkyl.

In some embodiments, R² is H. In some embodiments, R² is not H.

In some embodiments, R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, or NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

In some embodiments, R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, or NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, —SH, cyano, (C₃-C₈)cycloalkyl, and NR^{g}R^{h}.

In some embodiments, R³ is H, (C₁-C₆)alkyl, (C₂-C₆)alkenyl, (C₂-C₆)alkynyl, or NR^{g}R^{h}, wherein any (C₁-C₆)alkyl, (C₂-C₆)alkenyl, and (C₂-C₆)alkynyl, is optionally substituted with one or more groups independently selected from the group consisting of halo, —SH, cyano, (C₃-C₈)cycloalkyl, and NR^{g}R^{h}.

In some embodiments, R³ is H or (C₁-C₆)alkyl optionally substituted with one or more groups independently selected from the group consisting of halo, —SH, cyano, (C₃-C₈)cycloalkyl, and NR^{g}R^{h}.

In some embodiments, R³ is H or (C₁-C₆)alkyl.

In some embodiments, R³ is H. In some embodiments, R³ is not H.

In some embodiments, R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—.

In some embodiments, R⁴ is 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—.

In some embodiments, R⁴ is R^{k}—C(═O)—, R^{k}—O—C(═O)—, R^{k}—S(O)₂—O—, R^{c}R^{d}NS(O)₂—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NS(O)₂—, or R^{e}S(O)₂NR^{f}—.

In some embodiments, R⁴ is R^{k}—C(═O)—, R^{k}—O—C(═O)—, or 1-ethylene that is substituted at the 2-position with R^{k}—C(═O)—, R^{k}—O—C(═O)—.

In some embodiments, R⁴ is R^{k}—S(O)₂—O—, R^{c}R^{d}NS(O)₂—, R^{e}S(O)₂NR^{f}—, or 1-ethylene that is substituted at the 2-position with R^{k}—S(O)₂—O—, R^{c}R^{d}NS(O)₂—, or R^{e}S(O)₂NR^{f}—.

In some embodiments, R⁴ is R^{k}—C(═O)— or R^{k}—O—C(═O)—.

In some instances, provided herein is a compound of Formula I, wherein R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}. In such cases, R⁴ can be R^{e}S(O)₂NR^{f}— and R^{e} can be R^{w}. In other instances, R⁴ is 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—. A compound of Formula I herein can also be a compound where R^{b} is H. In other cases, R^{b} is X—Y. In such cases, X can be (C₁-C₆)alkyl, (C₂-C₆)alkenyl, or (C₁-C₆)alkynyl, which (C₁-C₆)alkyl, (C₂-C₆)alkenyl, or (C₁-C₆)alkynyl is optionally substituted with oxo.

In some embodiments, R^{a} is H, (C₁-C₆)alkyl, (C₂-C₆)alkenyl, (C₂-C₆)alkynyl, (C₁-C₆)alkanoyl, or (C₁-C₆)alkoxycarbonyl, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h}.

In some embodiments, R^{a} is H, (C₁-C₆)alkyl, (C₂-C₆)alkenyl, or (C₂-C₆)alkynyl, wherein any (C₁-C₆)alkyl, (C₂-C₆)alkenyl, or (C₂-C₆)alkynyl is optionally substituted with one or more groups independently selected from the group consisting of halo, —SH, cyano, oxiranyl, (C₃-C₈)cycloalkyl, (C₁-C₆)alkoxy, (C₁-C₆)alkylthio, and NR^{g}R^{h}.

In some embodiments, R^{a} is H, (C₁-C₆)alkyl, (C₂-C₆)alkenyl, or (C₂-C₆)alkynyl, wherein any (C₁-C₆)alkyl, (C₂-C₆)alkenyl, or (C₂-C₆)alkynyl is optionally substituted with one or more groups independently selected from the group consisting of halo, —SH, cyano, and NR^{g}R^{h}.

In some embodiments, R^{a} is H or (C₁-C₆)alkyl optionally substituted with one or more groups independently selected from the group consisting of halo, —SH, cyano, and NR^{g}R^{h}.

In some embodiments, R^{a} is H or (C₁-C₆)alkyl.

In some embodiments, R^{a} is H. In some embodiments, R^{a} is not H.

In some embodiments, provided herein is a compound of Formula I, wherein
ring A is selected from the group consisting of rings i, j, k, l, u, and w;
R¹, R², and R³ can independently be H or optionally substituted (C₁-C₆)alkyl;
R⁴ is R^{k}—C(═O)—, R^{k}—O—C(═O)—, or R^{k}—S(O)₂—O—;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some embodiments, provided herein are compounds of Formula I in which ring A is selected from the group consisting of:

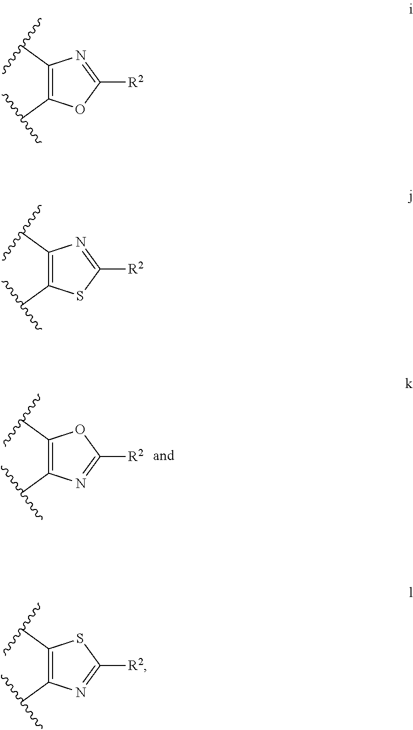

wherein R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}. In some instances, R² is H or optionally substituted (C₁-C₆)alkyl. In some instances, ring A is ring i. In some instances, ring A is ring j. In some instances, ring A is ring k. In some instances, ring A is ring 1.

In instances in which ring A is ring j, a compound can be of Formula II:

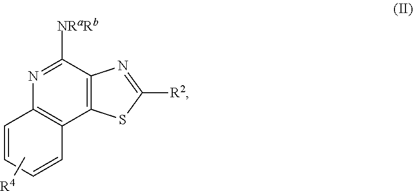

or a pharmaceutically acceptable salt thereof,
wherein:
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, or R^{k}—S(O)₂—O—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some embodiments of Formula II:

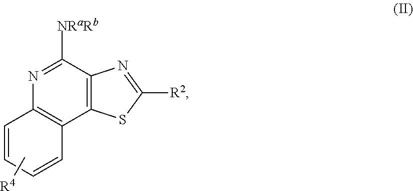

or a pharmaceutically acceptable salt thereof,
wherein:
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)— or R^{k}—O—C(═O)—;
R^{a} is H or optionally substituted (C₁-C₆)alkyl;
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

In some embodiments, provided herein is a compound of Formula II, wherein:
R² is H or optionally substituted (C₁-C₆)alkyl;
R⁴ is R^{k}—C(═O)— or R^{k}—O—C(═O)—;
R^{a} is H or optionally substituted (C₁-C₆)alkyl;
R^{b} is H or X—Y;
R^{k} is H or optionally substituted (C₁-C₆)alkyl;
X is a linking group; and
Y is an antigen or maleimide.

In some embodiments, and with reference to Formula II, R² is optionally substituted (C₁-C₆)alkyl; R^{k}—O—C(═O)—, wherein R^{k} is optionally substituted (C₁-C₆)alkyl; R^{a} is H; and R^{b} is H or X—Y, with X being a linking group, and Y being an antigen or maleimide.

Hence, in some embodiments, provided herein is a compound of Formulas I or II having the following structure of compound 1:

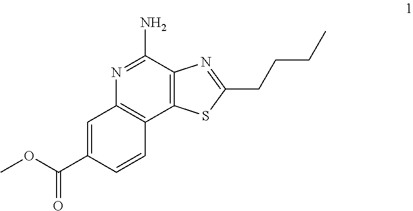 [SMILES: CCCCc1nc2c(N)nc3cc(C(=O)OC)ccc3c2s1]

In some embodiments, provided herein are compounds of Formula I in which ring A is:

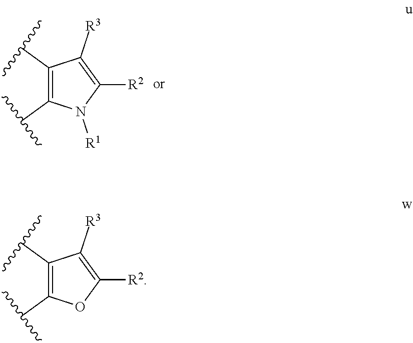

wherein
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}, as described herein for Formula I.

Hence, in same aspects, provided herein are compounds of Formulas III and IV, respectively:

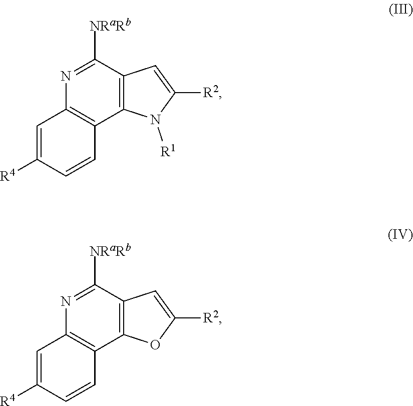

wherein,
R¹, R², and R³ can independently be H or (C₁-C₆)alkyl that can be optionally substituted with (C₁-C₃)alkyl, substituted or unsubstituted (C₃-C₈)cycloalkyl or substituted or unsubstituted aryl;
R⁴ is R^{k}—C(═O)—, R^{k}—O—C(═O)—, or R^{k}—S(O)₂—O—;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
R^{a} is H or optionally substituted (C₁-C₆)alkyl;
R^{b} is H or X—Y;
X is a linking group; and
Y is an antigen or maleimide.

Hence, in some embodiments, a compound herein is selected from the group consisting of:

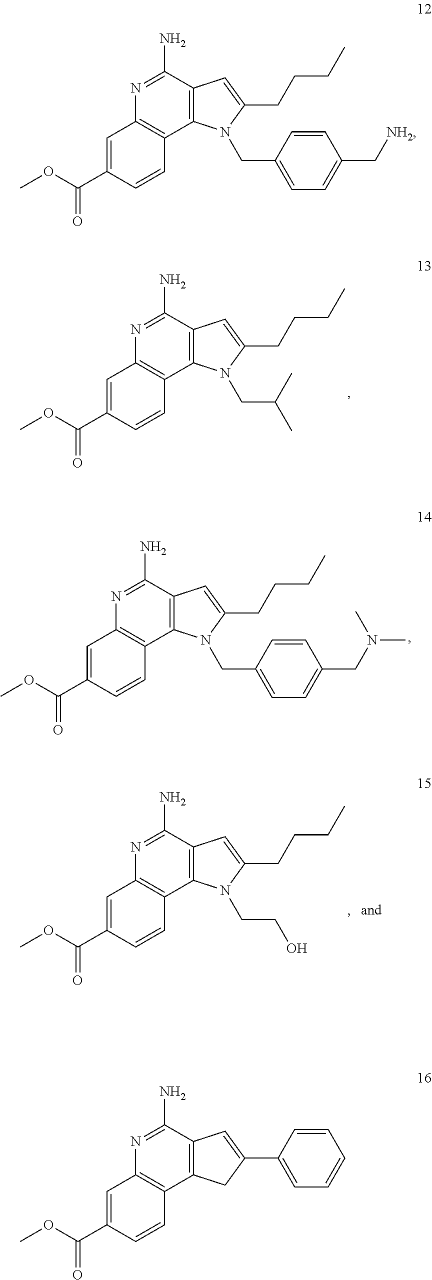

In some embodiments, a compound of the present disclosure, e.g., a compound of any one of Formulae I-IV, can be capable of binding to a toll-like receptor (TLR). In some embodiments, the binding of such compound to a TLR can exhibit an agonist effect on the TLR. In some embodiments, the binding of a compound to a TLR can exert an immunostimulatory effect.

Compound Synthesis

Processes for preparing compounds of Formulae I-IV are provided as further embodiments of the present disclosure and are illustrated by the following procedures in which the meanings of the generic radicals are as given above unless otherwise qualified. Certain compounds of Formulae I-IV can be useful as intermediates for preparing other compounds of Formulae I-IV. In cases where compounds are sufficiently basic or acidic, a pharmaceutically acceptable salt of a compound of Formulae I-IV can be useful as an intermediate for isolating or purifying a compound of Formulae I-IV.

Conventional synthetic approaches toward the tricyclic A-B-C heterocycles as shown in Formulae I-IV can be based off substituted quinoline scaffolds. However, such routes can be limited in their ability to produce versatile functional group patterns on the tricyclic A-B-C heterocycles due to harsh reaction conditions that may be required. Moreover, conventional synthetic approaches may not yield the 5N-oxide intermediate in the presence of electron-withdrawing groups as further shown below.

Thus, in some embodiments, provided herein are improved synthetic routes toward such tricyclic A-B-C heterocycles shown in Formulae I-IV, such as thiazoquinolines, that can allow installation of various substituents (e.g., R⁴) on ring C of Formula I. In various aspects, substituted quinoline compounds of the present disclosure, including those of Formulae I-IV, can be produced according to SCHEME 1 below:

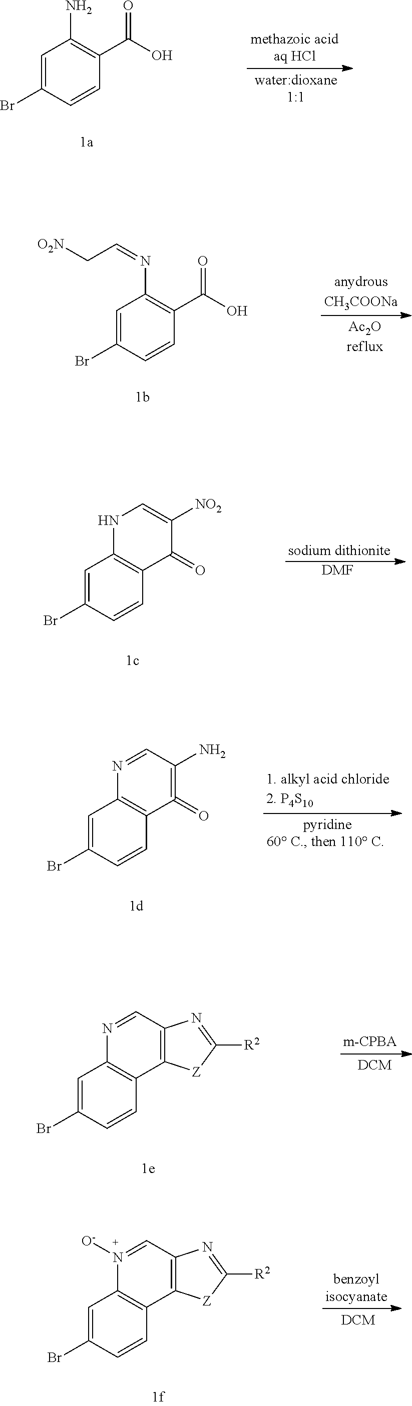

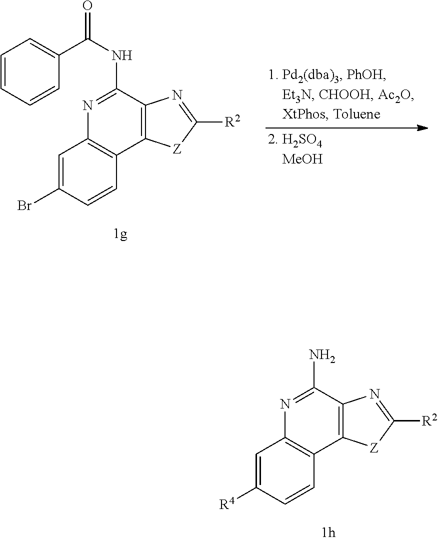

wherein:
Z is S, O, or —NR^{f},
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo.

SCHEME 2 below shows a variation on SCHEME 1 above, including alternative compound substitutions:

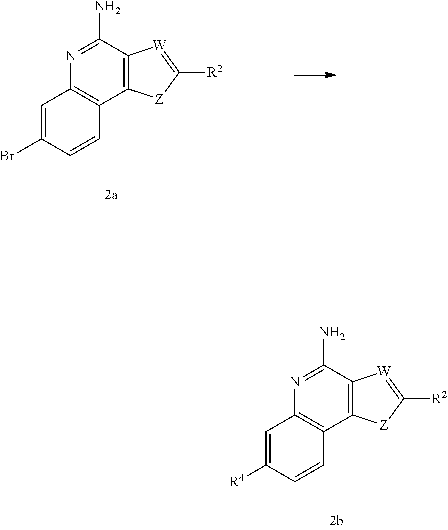

wherein:
W is N or CH;
Z is S, O, or —NR^{f};
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—.
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo.

In some embodiments and according to reaction SCHEME 1 and SCHEME 2 above, R² is (C₁-C₆)alkyl.

In some embodiments and according to reaction SCHEME 1 and SCHEME 2 above:
R⁴ is aryl, (C₂-C₆)alkynyl, vinyl, cyano, R^{k}—C(═O)—, R^{m}RⁿNC(═O)—, or R^{k}—O—C(═O)—;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl; and
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl.

Alternatively, 7-bromo-3-nitroquinolin-4(1H)-one in SCHEME 1 above can be synthesized as shown in SCHEME 3 below:

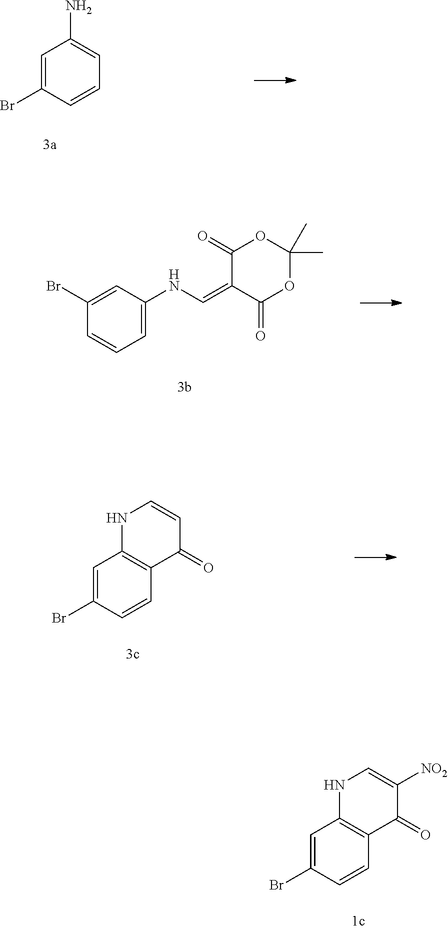 [SMILES: Nc1cccc(Br)c1 ; CC1(C)OC(=O)C(=CNc2cccc(Br)c2)C(=O)O1 ; O=c1cc[nH]c2cc(Br)ccc12 ; O=c1c([N+](=O)[O-])c[nH]c2cc(Br)ccc12]

In some embodiments, provided herein is a procedure that can begin with the installation of a bromo group to the thiazoloquinoline using a modified Niementowski reaction to produce the desired 3-amino-4-hydroxy-7-bromoquinoline. Cyclization to such 7-bromo-thiazoloquinoline can then be afforded by first forming the primary amide at the 3-amino position followed by dehydration using phosphorous pentasulfide in pyridine. The oxidation of the N5 position using 3-chloroperbenzoic acid in dichloromethane in the presence of the C7-bromo group may then follow in good yields (e.g., >70%) to obtain the N5-oxide. In such instances, no or only very small amounts of the N3 oxidation product may be present. The 4-amino group can then be installed as a benzamido surrogate via a 1,3-dipolar cycloaddition reaction using the dipolarophile benzoyl isocyanate with carbon dioxide being released as a byproduct. The final product can then be obtained in a two-step process that generates the C7-phenyl ester using Pd₂(dba)₃ with a catalyst, e.g., XPhos, for the alkoxycarbonylation followed by acid catalyzed hydrolysis and transesterification to yield the target thiazoquinoline compound as shown in

In some embodiments, further provided herein are synthetic methods for various substituted quinolines capable of interacting with one or more TLRs, such as TLR-7 and/or TLR-8. In some aspects, provided herein is a synthetic route for substituted pyrroloquinolines, e.g., as shown in SCHEME 4:

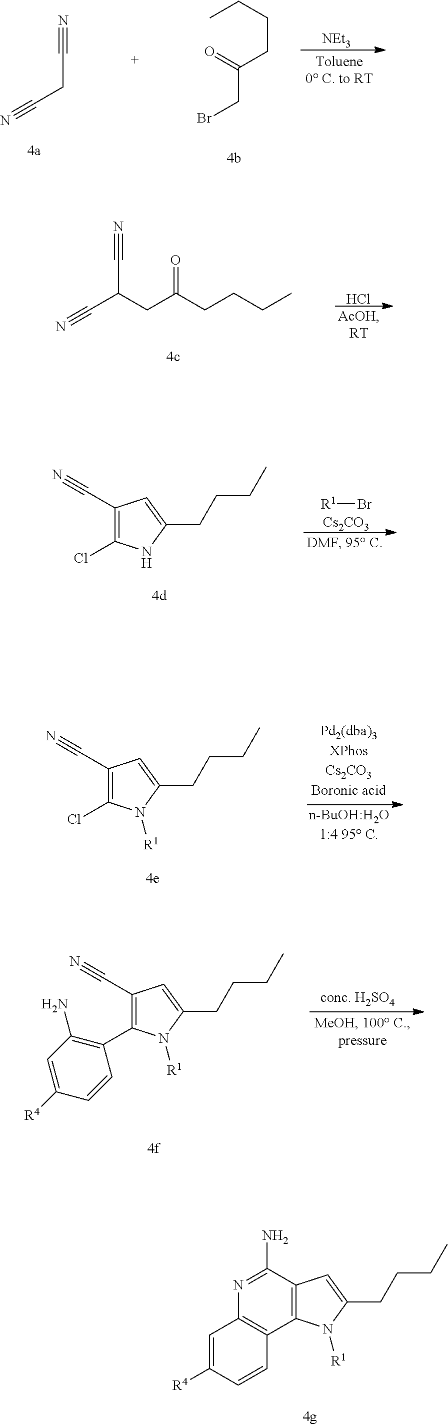

wherein:
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;

Other embodiments of this disclosure provide a synthetic route for substituted furoquinolines, e.g., as shown in SCHEME 5:

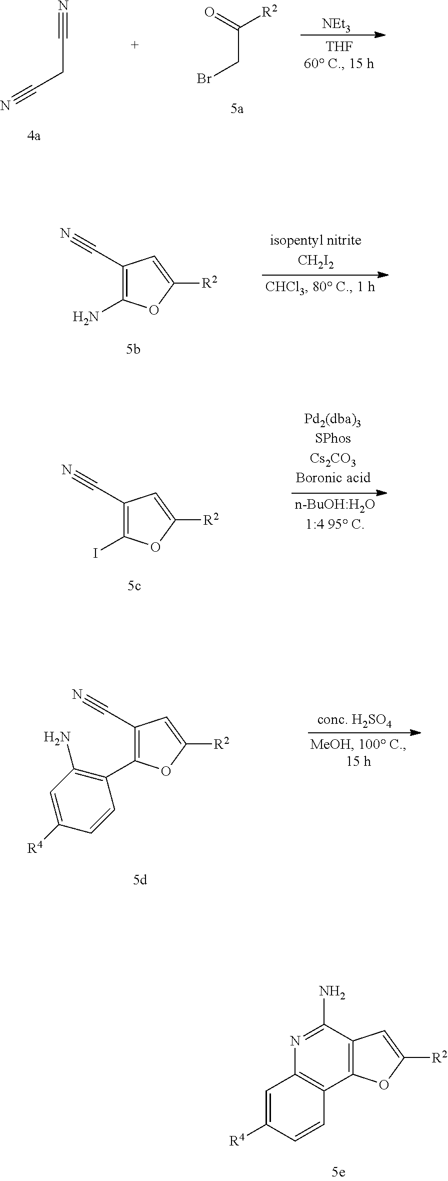

wherein:
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo.

In some embodiments, substituents R¹, R², and R⁴ of SCHEMES 1-5 are independently selected from the group consisting of H, (C₁-C₆)alkyl, aryl, heteroaryl, and (C₃-C₈)cycloalkyl, all of which (except H) can be optionally substituted with (C₁-C₃)alkyl, substituted or unsubstituted (C₃-C₈)cycloalkyl or substituted or unsubstituted aryl. In some embodiments, SCHEMES 1-5 can be used to synthesize compounds of Formulae I-IV, e.g., compounds 1-26 either as intermediates or final products.

III. Linkers

In some embodiments, provided herein are molecules that can comprise a compound of any one of Formulae I-IV and a linker. In such cases, the linking group X of, e.g., Formula I herein, can join the remainder of the compound of Formulae I-IV to another molecule Y. Such other molecule Y can comprise or consist of a small molecule, a peptide, a protein, a nucleic acid, or a combination thereof. In some cases, X—Y can comprise a maleimide moiety coupled to an antigen Y. In various aspects, the nature of the linking group X may not be critical, provided the resulting antigen conjugate retains the biological properties of the unconjugated antigen.

In some embodiments, a linker X can have a molecular weight of from about 20 daltons to about 20,000 daltons, from about 20 daltons to about 5,000 daltons, from about 20 daltons to about 1,000 daltons, or from about 20 daltons to about 200 daltons. In some cases, X can have a length of about 5 angstroms to about 60 angstroms. In some embodiments, a linker X separates an antigen from the remainder of the compound of Formulae I-IV by about 5 angstroms to about 40 angstroms, inclusive, in length.

A linker moiety X of the present disclosure can comprise or consist of a divalent, branched or unbranched, saturated or unsaturated, hydrocarbon chain, having from about 2 to about 25 carbon atoms, wherein one or more (e.g. 1, 2, 3, or 4) of the carbon atoms is optionally replaced by (—O—), and wherein the chain is optionally substituted on a carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy. In another embodiment, a linker X comprises or consists of a polyethyleneoxy chain. Such polyethyleneoxy chain can comprise or consist of 2, 3, 4, 5, 6, 7, 8, 9, or 10 repeating ethyleneoxy units.

In some instances, a linker moiety X can be a divalent radical formed from a protein. In some instances, a linker moiety X can be a divalent radical formed from a peptide. In some instances, a linker moiety X can be a divalent radical formed from an amino acid.

In some embodiments, a linker moiety X can comprise or consist of a molecule selected from the group consisting of:

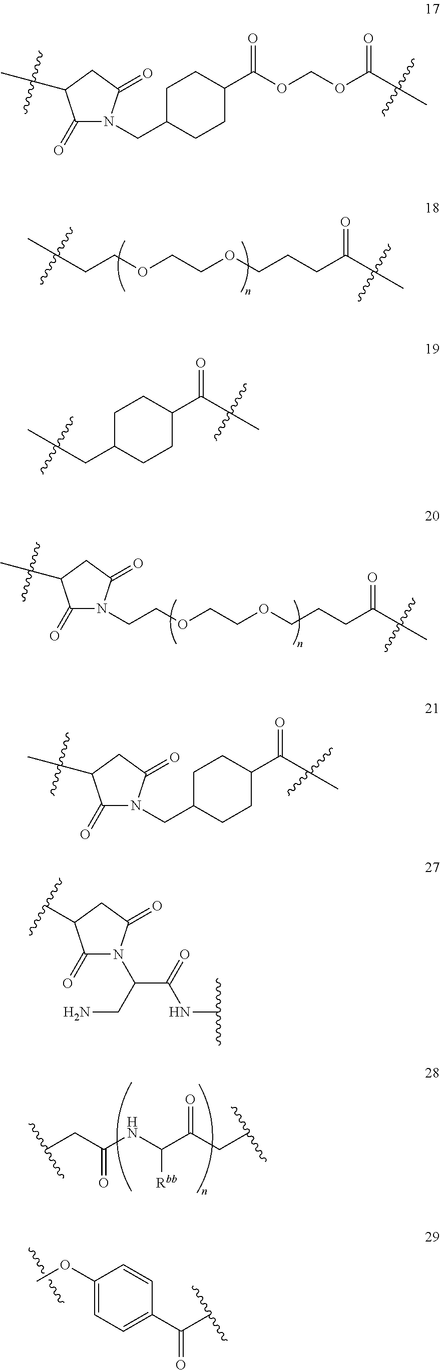

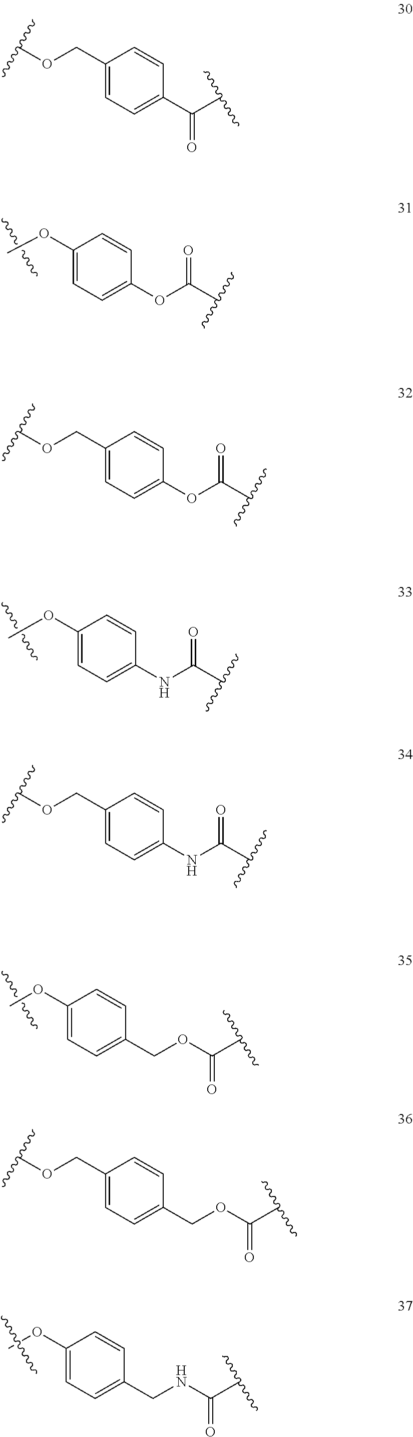

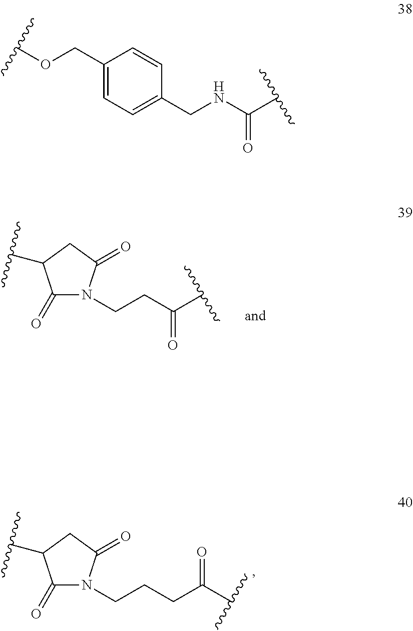

wherein n is 2, 3, 4, 5, or 6, and
wherein R^{bb} is an amino acid side chain.

In some embodiments, a linker moiety X can comprise or consist of a molecule selected from the group consisting of:

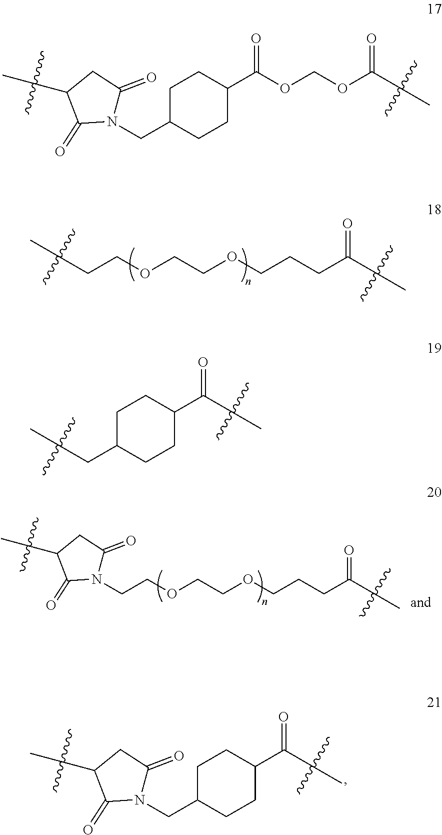

and wherein n is 2, 3, 4, 5, or 6.

In some embodiments, a linker moiety X can comprise or consist of a molecule selected from the group consisting of:

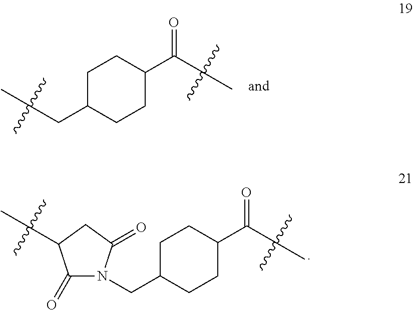 [SMILES: *CC1CCC(C(*)=O)CC1 ; *C(=O)C1CCC(CN2C(=O)CC(*)C2=O)CC1]

IV. Conjugates

The present disclosure further provides conjugates comprising one or more compound(s) of any one of Formulae I-IV coupled to another molecule Y.

As described herein, in some cases, such one or more compound(s) of any one of Formulae I-IV can be coupled to the other molecule Y via a linker. In some cases, the other molecule Y can be a biomolecule such as a peptide or a protein (e.g., an antibody or antigen-binding fragment thereof). Such conjugate can be a molecule of Formula V, which is equal to Formula I with R^{b} is X—Y, and X being a linking group and Y being a peptide, a protein, or maleimide.

In some instances, a compound of Formulae I-IV herein is coupled to a biomolecule Y, such as a peptide or a protein. In some cases, the biomolecule Y is an antibody. In other cases, the biomolecule Y is an antigen. Hence, provided herein are conjugates comprising one or more compound(s) of any one of Formulae I-IV coupled to an antigen Y via a linker X as described herein.

V. Pharmaceutical Compositions

Provided herein are pharmaceutical compositions comprising a one or more compounds of Formulae I-IV and/or one or more conjugates according to the present disclosure. In some instances, administration of a compound of Formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example, calcium) salts of carboxylic acids can also be made.

The compound(s) of Formula I can be formulated as pharmaceutical compositions and administered to a mammalian host, such as a human patient, in a variety of forms adapted to the chosen route of administration, e.g., orally or parenterally, by intravenous, intramuscular, topical or subcutaneous routes. Thus, the presently disclosed compounds may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations can contain at least 0.1% of active compound. The percentage of the compositions and preparations may be varied and may conveniently be between about 2% to about 60% of the weight of a given unit dosage form. The amount of active compound in such therapeutically useful compositions is such that an effective dosage level (e.g., when measured systemically and/or locally post-administration) will be obtained.

A tablet, troche, pill, capsule, and the like comprising one or more compounds of Formula I may further contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Generally, any material used in preparing any unit dosage form should be pharmaceutically acceptable and substantially non-toxic in the amounts employed. In addition, the active compound may be incorporated into sustained-release preparations and devices.

A compound of the present disclosure (e.g., those of any one of Formulae I-IV) may also be administered intravenously or intraperitoneally by infusion or injection. Solutions of such compound(s) or its pharmaceutically acceptable salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, e.g., when they are liquids. However, it will generally be desirable to administer them to the skin as compositions or formulations, in combination with a dermatologically acceptable carrier, which may be a solid or a liquid. Useful solid carriers include finely divided solids such as talc, clay, microcrystalline cellulose, silica, alumina, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the compound(s) of Formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). Useful dosages of the compound(s) of Formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound disclosed herein, or an active pharmaceutically acceptable salt or derivative thereof, for use in treatment may vary not only with the particular salt selected but also with the route of administration, the nature of the condition being treated and the age and condition of the patient and will be ultimately at the discretion of the attendant physician or clinician. The desired dose may conveniently be presented in a single dose or as divided doses administered at appropriate intervals, for example, as two, three, four or more sub-doses per day. The sub-dose itself may be further divided, e.g., into several discrete loosely spaced administrations; such as multiple inhalations from an insufflator or by application of a plurality of 4 drops into the eye.

As described herein, provided herein are pharmaceutical compositions comprising one or more compounds of Formula I, and/or conjugate(s) thereof, and prepared for therapeutic and prophylactic use in a mammal (e.g., a human). In some cases, such pharmaceutical composition is formulated into an oral dosage form or into a dosage form for injection. Exemplary dosage forms are shown below in TABLE 1.

TABLE 1
Dosage Forms according to embodiments of this disclosure

| Tablet 1 | Tablet 2 | Capsule | Injection 1 | Injection 2 | Aerosol |
|---|---|---|---|---|---|
| (mg/tablet) | (mg/tablet) | (mg/capsule) | (mg/mL) | (mg/mL) | (mg/can) |
| Compound of | Compound of | Compound of | Compound of | Compound of | Compound of |
| Formula I | Formula I | Formula I | Formula I - | Formula I - | Formula I (20) |
| (100) | (20) | (10) | free acid (1.0) | free acid (10) | |
| Lactose (77.5) | Microcrystalline | Colloidal | Dibasic | Monobasic | Oleic acid (10) |
| | cellulose | silicon | sodium | sodium | |
| | (410) | dioxide (1.5) | phosphate (12) | phosphate (0.3) | |
| Povidone (15) | Starch (50) | Lactose | Monobasic | Dibasic sodium | Trichloromono- |
| | | (465.5) | sodium | phosphate (1.1) | fluoromethane |
| | | | phosphate | | (5,000) |
| | | | (0.7) | | |
| Croscarmellose | Sodium starch | Pregelatinized | Sodium | Polyethylene | Dichlorodifluoro- |
| sodium (12) | glycolate (15) | starch (120) | chloride (4.5) | glycol 400 | methane (10,000) |
| | | | | (200) | |
| Microcrystalline | Magnesium | Magnesium | 1.0N | 1.0N | Dichlorotetrafluoro- |
| cellulose | stearate (5) | stearate (3.0) | Sodium | Sodium | ethane (5,000) |
| (92.5) | | | hydroxide | hydroxide | |
| | | | solution | solution | |
| | | | (pH | (pH | |
| | | | adjustment to | adjustment to | |
| | | | 7.0-7.5) (q.s.) | 7.0-7.5) (q.s.) | |
| Magnesium | | | Water for | Water for | |
| stearate (3.0) | | | injection (q.s. | injection (q.s. | |
| | | | ad 1 mL) | ad 1 mL) | |

The compositions comprising a compound of Formulae I-IV, a conjugate, and/or a pharmaceutically acceptable salt thereof, can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formulae I-IV, or a pharmaceutically acceptable salt thereof) calculated to produce the desired therapeutic effect, in association with a suitable pharmaceutical excipient.

An effective amount of the active material (i.e., a compound or conjugate of Formulae I-IV, or a pharmaceutically acceptable salt of any of the foregoing) is ordinarily supplied at a dosage level of from about 0.01 mg/kg to about 1000 mg/kg of body weight per day, or any range therein. In some cases, the range is from about 0.05 to about 500 mg/kg of body weight per day, or any range therein. In some cases, from about 0.1 to about 250 mg/kg of body weight per day, or any range therein. In some cases, from about 0.1 to about 100 mg/kg of body weight per day, or any range therein. In an example, the range can be from about 0.1 to about 50.0 mg/kg of body weight per day, or any amount or range therein. In another example, the range can be from about 0.01 to about 15.0 mg/kg of body weight per day, or any range therein. In yet another example, the range can be from about 0.05 to about 7.5 mg/kg of body weight per day, or any amount to range therein. In yet another example, the range can be from about 0.1 to about 5.0 mg/kg of body weight per day, or any amount to range therein. Pharmaceutical compositions comprising a compound of Formulae I-IV, or a pharmaceutically acceptable salt of any of the foregoing, can be administered on a regimen of 1 to 4 times per day or in a single daily dose.

VI. Methods of Use

Provided herein are methods of using one or more compounds of Formulae I-IV, conjugate(s) thereof, and/or pharmaceutical compositions comprising such compounds, for the treatment of a disease or pathological condition in a subject in need thereof. In some embodiments, such methods can comprise administering to a subject in need thereof a compound of Formulae I-IV, conjugate, or pharmaceutical composition of this disclosure in a therapeutically effective amount to thereby treat such disease or condition in the subject. In some cases, the subject is a mammal such as a human or a rodent. Diseases and/or conditions that can be treated using the compounds of the present disclosure include but are not limited to cancer, a viral or a bacterial infection.

Cancers, including, but not limited to, a tumor, metastasis, or other disease or disorder characterized by abnormal cells that are characterized by uncontrolled cell growth in some embodiments are treated or inhibited by administration of a compound of the present disclosure, or a conjugate thereof as disclosed herein. In some embodiments, the subject has previously undergone treatment for the cancer. In some embodiments, the prior treatment is surgery, radiation therapy, administration of one or more anticancer agents, or a combination of any of the foregoing.

In any of the methods described herein, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangio-endothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

The methods provided herein comprise methods of treating one or more disease(s) in a subject in need thereof. Some embodiments provide a method of treating cancer in a subject in need thereof, comprising administering to the subject a therapeutically effective amount a compound of Formulae I-IV, a pharmaceutically acceptable salt thereof, a conjugate thereof, or a pharmaceutical composition thereof. Some embodiments provide a method of inducing an anti-tumor immune response in a subject in need thereof, comprising administering to the subject a therapeutically effective amount a compound of Formulae I-IV, a pharmaceutically acceptable salt thereof, a conjugate thereof, or a pharmaceutical composition thereof.

Some embodiments herein provide a method of treating cancer in a subject in need thereof, comprising administering to the subject a therapeutically effective amount a compound of Formulae I-IV, or a pharmaceutically acceptable salt thereof, or conjugate thereof, in combination with another anticancer therapy (e.g., surgery and radiation therapy) and/or anticancer agent (e.g., an immunotherapy such as nivolumab or pembrolizumab). Compounds of Formulae I-IV, or conjugate(s) thereof, can be administered to the subject before, during, or after administration of the anticancer therapy and/or anticancer agent. In some embodiments, the compounds of Formulae I-IV described herein can be administered to the subject following treatment with radiation and/or after surgery.

Some embodiments provide a method for delaying or preventing acquired resistance to an anticancer agent, comprising administering to the subject a therapeutically effective amount a compound of Formulae I-IV, a pharmaceutically acceptable salt thereof, or conjugate thereof, to a patient at risk for developing or having acquired resistance to an anticancer agent. In some embodiments, the patient is administered a dose of the anticancer agent (e.g., at substantially the same time as a dose of the compound of Formulae I-IV, a pharmaceutically acceptable salt thereof, or conjugate thereof is administered to the patient).

Some embodiments provide a method of delaying and/or preventing development of cancer resistant to an anticancer agent in a subject, comprising administering to the subject a therapeutically effective amount a compound of Formulae I-IV, a pharmaceutically acceptable salt thereof, or conjugate thereof, before, during, or after administration of a therapeutically effective amount of the anticancer agent.

Compounds of Formulae I-IV, and/or conjugates thereof, are useful for inhibiting the multiplication of a cancer cell, causing apoptosis in a cancer cell, for increasing phagocytosis of a cancer cell, and/or for treating cancer in a subject in need thereof. In some embodiments, the cancer is as described herein. In some embodiments, the subject has previously undergone treatment for the cancer. In some embodiments, the prior treatment is surgery, radiation therapy, administration of one or more anticancer agents, or a combination of any of the foregoing. In some embodiments, the subject has discontinued a prior therapy, for example, due to unacceptable or unbearable side effects, wherein the prior therapy was too toxic, or wherein the subject developed resistance to the prior therapy. Some embodiments provide a method for delaying or preventing a disease or disorder, comprising administering to the subject a therapeutically effective amount of a compound of Formulae I-IV, or a pharmaceutically acceptable salt thereof, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the compound of Formulae I-IV, a pharmaceutically acceptable salt thereof, or a conjugate thereof, and the vaccine are administered via the same route (for example, the compound of Formulae I-IV, or a pharmaceutically acceptable salt thereof, and the vaccine are both administered subcutaneously). In some embodiments, the compound of Formulae I-IV, a pharmaceutically acceptable salt thereof, or conjugate thereof and the vaccine are administered via different routes. In some embodiments, the vaccine and the compound of Formulae I-IV, or a pharmaceutically acceptable salt thereof, are provided in a single formulation. In some embodiments, the vaccine and the compound of Formulae I-IV, or a pharmaceutically acceptable salt thereof, are provided in separate formulations. In some embodiments, the compounds of Formulae I-IV described herein are present in the form of a salt when used for treatment. In some embodiments, the salt is a pharmaceutically acceptable salt.

VI. Compound Activity and Testing

Provided herein are various experimental procedures for synthesizing, evaluating, and using the compounds (e.g., those of Formulae I-IV) and conjugates of the present disclosure.

Screening Binding Ability of Compounds to TLR-7/8-expressing Cells: Toll-like receptor (TLR)-7 or TLR-8 positive cell lines and HEK-Blue TLR cells can be used in the screening assay. HEK-Blue TLR cells can be engineered HEK293 cells that stably express a TLR gene and an inducible NF-kB-SEAP (secreted embryonic alkaline phosphase) reporter gene. Binding of ligand(s) (e.g., a compound of Formulae I-IV) with the TLR in HEK-Blue cells can induce SEAP that has pNPP as substrate of phosphase to become blue. In some cases, a compound of the present disclosure, e.g., at about 20 nmol/ml or about 5.2 nmol/ml concentration, can be added in triplicate to HEK-Blue-TLR7 or TLR8 cells, cultured at 37° C. and 5% CO₂ condition. After a time period, e.g., 24 hours later, a volume (e.g., 5 μL) of supernatant of each experiment can be mixed with about 200 μl of pNPP-included detection medium. After one hour, SEAP activity can be read out as OD at 650 nm with a microplate reader to determine binding affinity of the compounds tested.

TLR7/8 Modulator NF-κB Reporter Assay: Human embryonic kidney (HEK) cells stably transfected with human TLR-7 or TLR-8 and an NF-κB-responsive secreted embryonic alkaline phosphatase (SEAP) gene (HEK-TLR-7 and -8) can be purchased, and can then be stimulated with 30 μM of compound (e.g., a compound of Formulae I-IV) in a 96-well plate in DMEM containing 10% FBS and 0.01% Normocin (InvivoGen) for about 24 h. Subsequently, a volume (e.g., about 20 μL) of the supernatant from each well can be incubated with Quanti-blue substrate solution (InvivoGen) at 37° C. for about 1 h and absorbance can be read at 650 nm wavelength.

Measurement of Proimflammatory Cytokines with Cytometric Bead Assay: Bone marrow derived dendritic cells (BMDC) can be generated by isolating a single cell suspension of marrow from the femur of C57BL/6 mice (6-8 weeks of age). Red blood cells can then be lysed with 0.83% NH₄Cl, 0.1% KHCO₃ and 0.009% 0.5 million cells can be seeded in each of well of a 6 well plate in complete RPMI media (Invitrogen, Grand Island, NY), supplemented with mouse 20 ng/ml Granulocyte-Macrophage Colony Stimulating Factor (PeproTech, Rocky Hill, NJ). Several days after culture, BMDC can be stimulated with 30 μM of compound (e.g., a compound of Formulae I-IV) for 3 days. Subsequently, a volume of about 25 μL of supernatant can be removed and assayed for TNFα, IL-12p40, IL-1β and IL-10 using a flow cytometric bead array according to the manufacturers' instructions (e.g., BD Bioscience, San Jose, CA). Controls can be performed using the addition of media and carrier with no drug.

Evaluating Effects on Human Monocyte-Derived Dendritic Cells (MoDC): MoDCs can be plated into 96-well plate and stimulated for about 48 hours with several (e.g., 5 or more) different concentration of one or more compounds of Formula I at various concentration, e.g., about 0.3, 0.5, 1.3, 5.2 and 20 nmol per ml cell medium, and performed in triplicate. For immunostaining and flow cytometry analysis, and about 48 hours after stimulation, the cells can be stained with anti-HLA-DR, CD11c, CD-86, CD80, CD83, CD8a, CD123, combinations thereof, and relevant isotype controls, followed by analysis, including cytokine analysis.

In Vivo Evaluation of IL-6 Levels: TLR7 mutant mice and C57BL/6j mice, 8-12 weeks old, can be used to introduce a TLR7 mutant gene to 129S1/Sv derived from CJ7 embryonic stem cells. The cell line can be backcrossed ten times to C57BL/6Ncr, with no TLR7 RNA expression detected in bone marrow-derived macrophages. A single cell suspension of splenocytes from C57BL/6j or TLR7 mutant mice can then be isolated. Splenocytes in culture medium can be treated in triplicate with various concentrations of one or more compounds of the present disclosure. Supernatant from the culture medium can be harvested, e.g., at 12 hours and 24 hours after treatment. A cytometric bead array (BD Biosciences, San Jose, CA) can be used for measurement of IL-6 level.

Proliferation of pmel CD8 and IFNγ Production: BMDC cells from C56BL/6j mice and single cell suspension of splenocytes from pmel mice can be treated in triplicate with about 3.5 μg of human gp100 peptide per well (gp100 peptide sequence: CALLAVGATKVPR-NQDWLGVSRQLRTK, GenScript, Piscataway, NJ) and test compound (e.g., a compound of Formula I) at concentration of about 10.4 nmol/ml. BMDC cell can then be washed twice with complete RPMI medium and followed by coculture with pmel CD8 splenocytes CFSE-labeled and isolated from pmel splenocytes with CD8+T Cell isolation Kit at a ratio of 1:3 of DC/CD8. Four days after coculture, supernatants can be harvested, followed by IFNγ measurement. The cell pellets can be washed and stained with fluorocore-labeled antibodies.

IL-2 Production of OT-I cells after Stimulation with a Compound of Formulae I-IV: Single cell suspension of C57BL/6j can be treated in triplicate with test compound (e.g., one of Formula I) at about 20.8 nmol/ml concentration and added with and without 15 μg of ovalbumin per well. Four days later, the cells can be washed twice with complete RPMI medium and cultured with isolated OT-I CD8 T cells. After four days coculture, the supernatant can be harvested and detected. CBA can be conducted for TL-2 production.

### EMBODIMENTS

For further illustration, additional non-limiting embodiments of the present disclosure are set forth below.

Embodiment P1: a compound of Formula I:

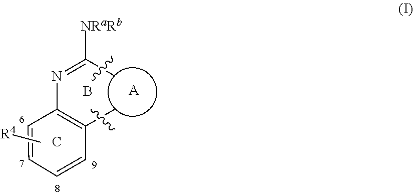

wherein:
the fused ring A is selected from the group consisting of:

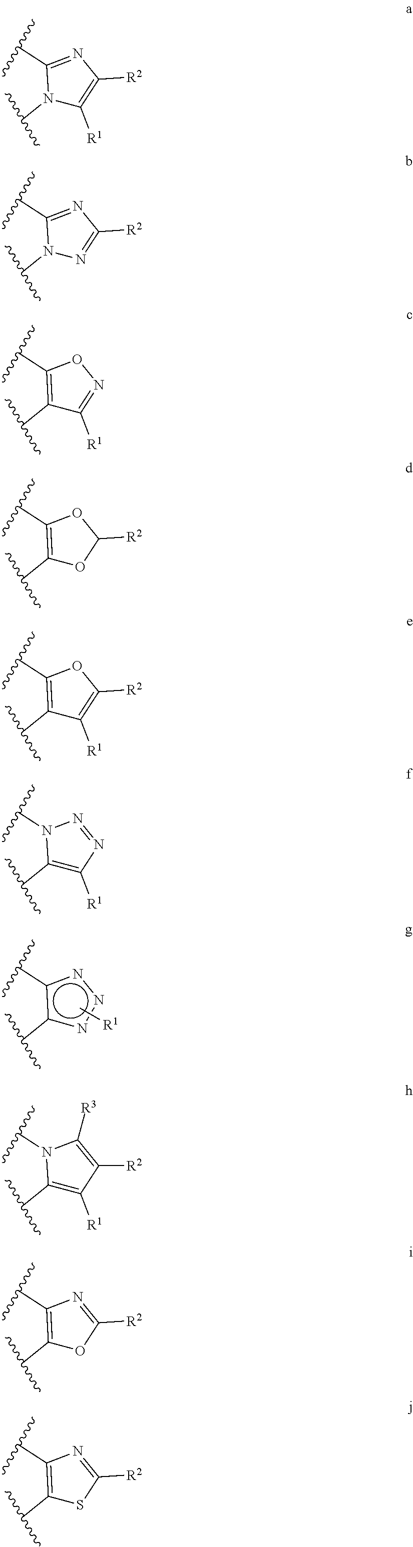

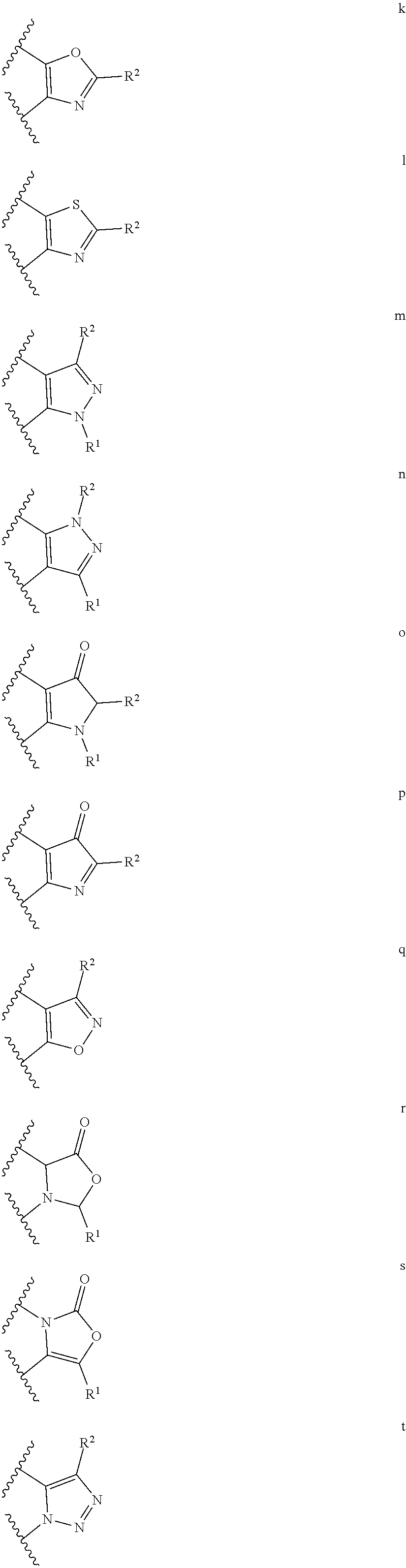

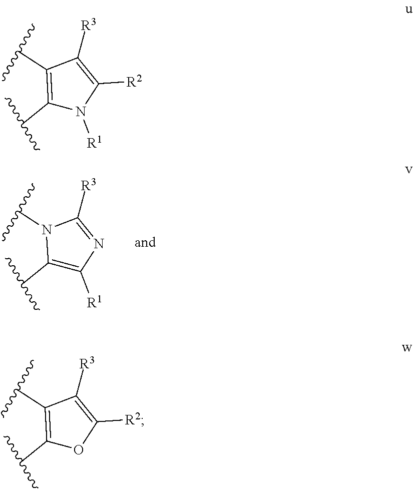

R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—; R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

Embodiment P2: the compound of Embodiment P1, wherein R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}.

Embodiment P3: the compound of Embodiment P1 or P2, wherein R⁴ is R^{e}S(O)₂NR^{f}— and R^{e} is R^{w}.

Embodiment P4: the compound of Embodiment P1 or P2, wherein R⁴ is R^{k}—C(═O)—, R^{k}—O—C(═O)—, or R^{k}—S(O)₂—O—.

Embodiment P5: the compound of Embodiment P1, wherein R⁴ is 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—.

Embodiment P6: the compound of any one of Embodiments P1, P2, P4, or P5, wherein R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, or (C₃-C₈)cycloalkyl.

Embodiment P7: the compound of any one of Embodiments P1-P6, wherein ring A is selected from the group consisting of:

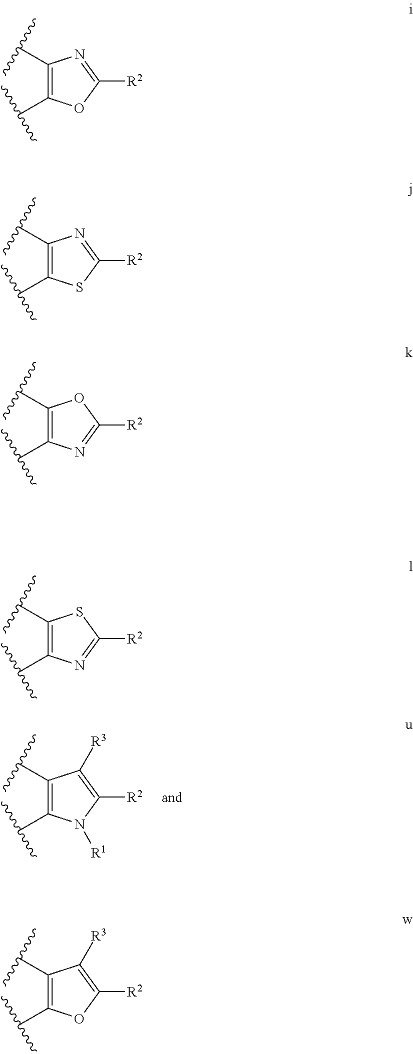

Embodiment P8: the compound of Embodiment P7, wherein R¹ and R³ are each independently H or optionally substituted (C₁-C₆)alkyl.

Embodiment P9: the compound of Embodiment 7P or P8, wherein R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

Embodiment P10: the compound of any one of Embodiments P1-P9, wherein ring A is selected from the group consisting of:

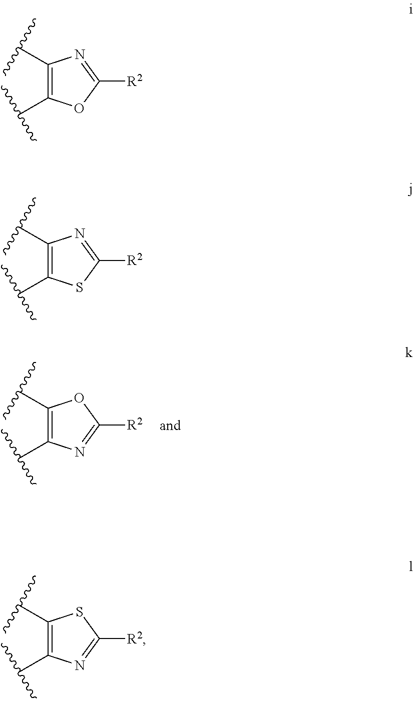

Embodiment P11: the compound of any one of Embodiments P1-P10, wherein R² is H or optionally substituted (C₁-C₆)alkyl.

Embodiment P12: the compound of any one of Embodiments P9-P11, wherein ring A is:

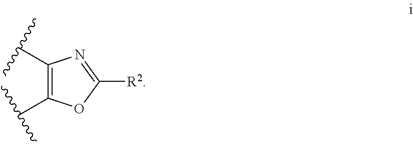

Embodiment P13: the compound of any one of Embodiments P9-P11, wherein ring A is:

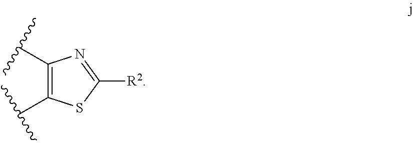

Embodiment P14: the compound of any one of Embodiments P9-P11, wherein ring A is:

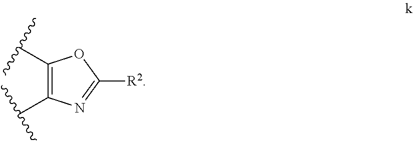

Embodiment P15: the compound of any one of Embodiments P9-P11, wherein ring A is:

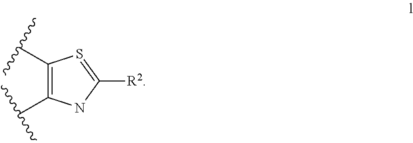

Embodiment P16: a compound of Formula II:

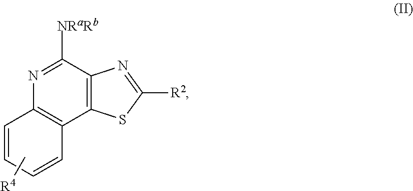

or a pharmaceutically acceptable salt thereof,
wherein:
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, or R^{k}—S(O)₂—O—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

Embodiment P17: the compound of Embodiment P16, wherein R² is H or optionally substituted (C₁-C₆)alkyl.

Embodiment P18: the compound of Embodiments P16 or P17, wherein R⁴ is R^{k}—C(═O)— or R^{k}—O—C(═O)—.

Embodiment P19: the compound of Embodiment P18, wherein R^{k} is H or optionally substituted (C₁-C₆)alkyl.

Embodiment P20: the compound of any one of Embodiments P16-P19, wherein R^{a} is H or optionally substituted (C₁-C₆)alkyl.

Embodiment P21: the compound of any one of Embodiments P16-P20, wherein R^{h} is H or X—Y.

Embodiment P22: the compound of any one of Embodiments P16-P21, having the formula of:

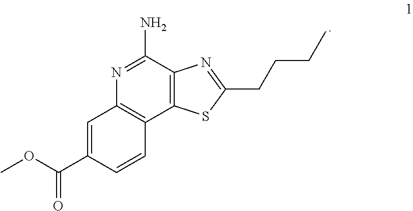 [SMILES: CCCCc1nc2c(N)nc3cc(C(=O)OC)ccc3c2s1]

Embodiment P23: the compound of any one of Embodiments P1-P7, wherein ring A is:

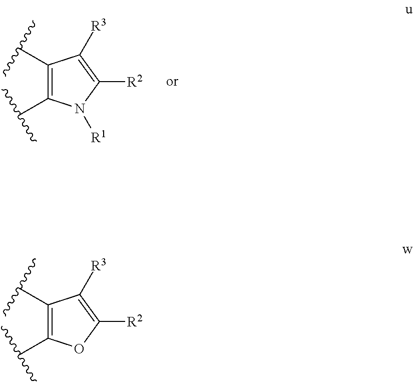

wherein
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}; and
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}

Embodiment P24: a compound of Formula III or Formula IV:

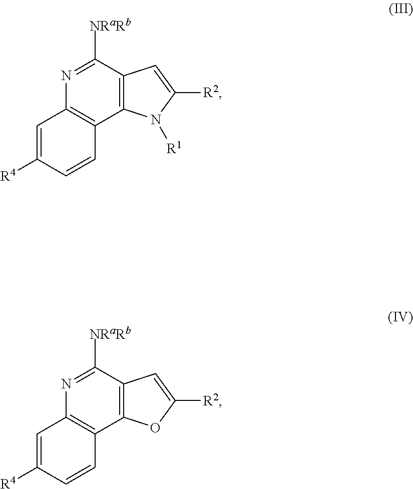

or a pharmaceutically acceptable salt thereof,
wherein:
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, or R^{k}—S(O)₂—O—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

Embodiment P25: the compound of Embodiment P24, wherein R¹ and R² are independently H or (C₁-C₆)alkyl that can be optionally substituted with (C₁-C₃)alkyl, substituted or unsubstituted (C₃-C₈)cycloalkyl or substituted or unsubstituted aryl.

Embodiment P26: the compound of Embodiment P24 or P25, wherein R⁴ is R^{k}—O—C(═O)—, or R^{k}—S(O)₂—O—.

Embodiment P27: the compound of Embodiment P24 or P25, selected from the group consisting of:

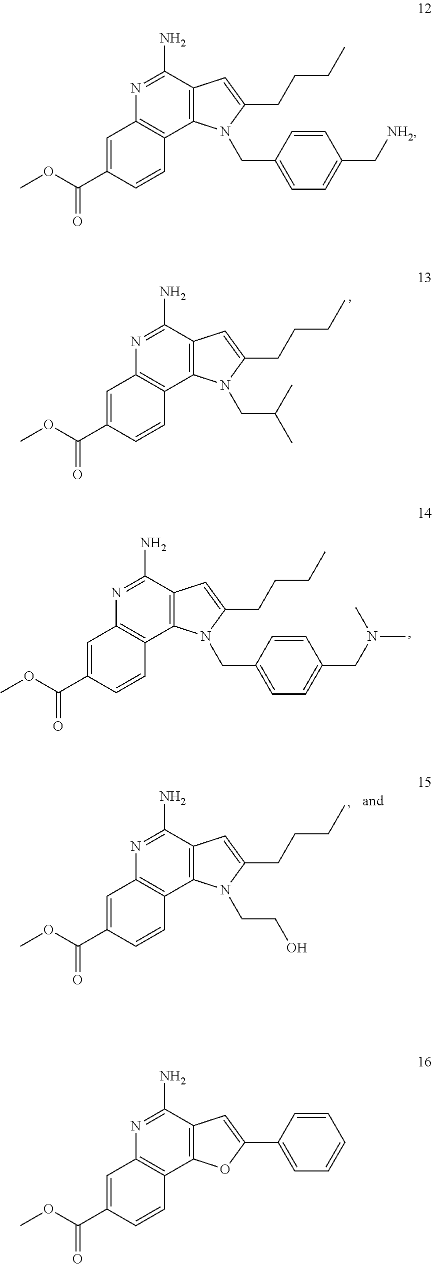

Embodiment P28: the compound of any one of Embodiments P1-P21 or P23-P26, wherein X is (C₁-C₆)alkyl, (C₂-C₆)alkenyl, or (C₁-C₆)alkynyl, which (C₁-C₆)alkyl, (C₂-C₆)alkenyl, or (C₁-C₆)alkynyl is optionally substituted with oxo.

Embodiment P29: the compound of any one of Embodiments P1-P21 or P23-P26, wherein X is selected from the group consisting of:

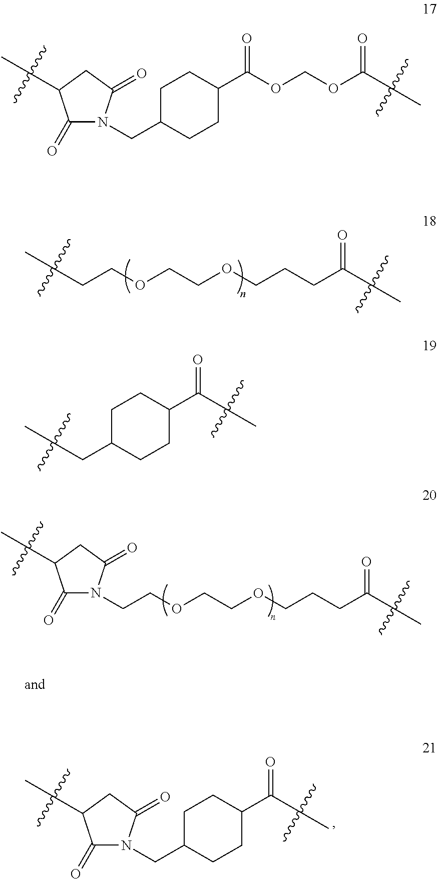

and wherein n is 2, 3, 4, 5, or 6.

Embodiment P30: the compound of any one of Embodiments P1-P21, P23-P26, or P28-P29, wherein Y is maleimide.

Embodiment P31: the compound of any one of Embodiments P1-P21, P23-P26, or P28-P29, wherein Y is an antigen.

Embodiment P32: the compound of Embodiment P31, wherein the antigen is associated with a bacterium or a virus, and wherein the virus is selected from the group consisting of an influenza virus, HIV, and HCV.

Embodiment P33: the compound of any one of Embodiments P1-P21, P23-P26, or P28-P29, wherein Y is an antigen associated with a tumor cell or a tumor cell lysate.

Embodiment P34: the compound of any one of Embodiments P31-P33, wherein Y is an antigen comprising a peptide sequence comprising cysteine, lysine, or both.

Embodiment P35: the compound of any one of Embodiments P1-P34, wherein the compound is any one of compounds 1 or 22-26.

Embodiment P36: a pharmaceutical composition comprising a compound according to any one of Embodiments P1-P35 and a pharmaceutically acceptable diluent or carrier.

Embodiment P37: a method of treating a pathological condition in an animal, comprising administering to the animal a compound according to any one of Embodiments P1-P35, or a pharmaceutically acceptable salt thereof, or a pharmaceutical composition according to Embodiment P36.

Embodiment P38: a method of stimulating an immune response in an animal, comprising administering to the animal a compound according to any one of Embodiments P1-P35, or a pharmaceutically acceptable salt thereof, or a pharmaceutical composition according to Embodiment P36.

Embodiment P39: a method of treating a cancer in an animal, comprising administering to the animal a compound according to any one of Embodiments P1-P35, or a pharmaceutically acceptable salt thereof, or a pharmaceutical composition according to Embodiment P36.

Embodiment P40: a method of synthesizing a compound of any one of Embodiments P1-P35, the method comprising performing a reaction according to any one of reaction SCHEMES 1-5.

### EXAMPLES

The following examples are given for the purpose of illustrating various embodiments of the disclosure and are not meant to limit the present disclosure in any fashion. The present examples, along with the methods described herein are presently representative of some embodiments, are exemplary, and are not intended as limitations on the scope of the disclosure. Changes therein and other uses which are encompassed within the spirit of the disclosure as defined by the scope of the claims will occur to those skilled in the art.

### Example 1

### General Synthetic Procedures

This example demonstrates the synthesis of compounds of Formulas I and II.

General Experimental Conditions: All solvents were used as received from commercial vendors, except for DMF, which was purified by passage through 2 alumina columns in a solvent purification system. 2-amino-4-bromobenzoic acid was purchased from AstaTech. Pd2(dba)₃ and triethylamine were purchased from Chem-Impex. XPhos was purchased from TCI America. Phosphorous pentasulfide was purchased from Acros Organics. All other general chemicals were purchased from Sigma-Aldrich, Macron, Alfa Aesar, Fisher Chemical, or were synthesized using the cited literature protocol. Thin layer chromatography (TLC) was performed on 0.25 mm hard-layer silica G plates; developed plates were visualized with a hand-held UV lamp. Silica used for chromatography was SiliaFlash® P60 from Silicycle. ¹H and ¹³C NMR spectra were recorded on a Varian 600 MHz or a Varian 400 MHz spectrometer in noted solvent; peaks are reported as chemical shift (multiplicity, J couplings in Hz, number of protons). High resolution mass spectra were obtained on an Agilent TOF II TOF/MS instrument equipped with either an ESI or APCI interface.

### Example 2

### Synthesis of 4-bromo-2-((2-nitroethylidene)amino)benzoic acid (3)

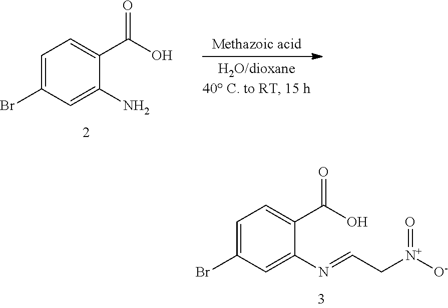 [SMILES: Nc1cc(Br)ccc1C(=O)O ; O=C(O)c1ccc(Br)cc1/N=C/C[N+](=O)[O-]]

2-Amino-4-bromobenzoic acid (2, 3.0 g, 13.89 mmol, 1 equiv.) was dissolved in a 1:1 mixture of water:dioxane (90 mL) at 50° C. The addition of the methazoic acid (40 mL, ˜28 mmol, ˜2 equiv) resulted in the immediate precipitation of an off-white solid. The solution was allowed to cool to 25° C. and stirred for 18 hours. The light yellow precipitate was filtered and rinsed with water until the filtrate was clear. The filtered solid was dried to constant mass in a vacuum oven. This crude yellow solid 3 was carried forward without characterization.

### Example 3

### Methazoic Acid Synthesis

Sodium hydroxide (6.72 g, 168 mmol) was dissolved in water (12 mL) and maintained at 25° C. To this aqueous sodium hydroxide, nitromethane (3 mL, 56 mmol) was added dropwise with rapid stirring and heated to 35° C. for 10 minutes. The reaction was cooled to 25° C. and another portion of nitromethane (3 mL, 56 mmol) was added while carefully maintaining the temperature at or below 35° C. After 10 minutes of rapid stirring, the reaction was heated to 50° C. for 5 minutes and then allowed to cool to 25° C. for 10 minutes. The reaction mixture was poured into cold water (50 mL) and then concentrated hydrochloric acid was added. Additional water was added until the total volume was 80 mL. The crude methazoic acid solution was used immediately in the next step or frozen (−20° C.) and then carefully thawed just before use in the reaction shown in EXAMPLE 2.

### Example 4

### Synthesis of 7-bromo-3-nitroquinolin-4-ol (4)

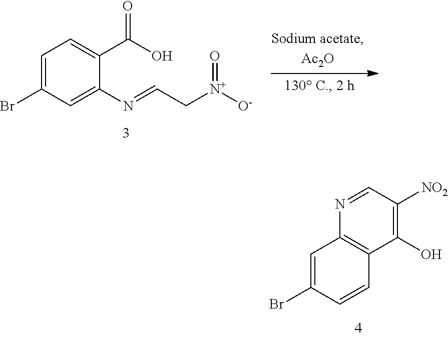 [SMILES: O=C(O)c1ccc(Br)cc1/N=C/C[N+](=O)[O-] ; O=[N+]([O-])c1cnc2cc(Br)ccc2c1O]

Crude 4-bromo-2-((2-nitroethylidene)amino)benzoic acid (3, 0.45 g, 1.57 mmol, 1 equiv.) was added to a flask with stir bar and acetic anhydride (5 mL). This suspension was heated to 100° C. for 45 minutes or until fully dissolved. Anhydrous sodium acetate was added to the reaction in one portion while vigorously stirring. The reaction was heated to reflux for 2 hours and then allowed to cool to 25° C. The contents of the flask were diluted with cooled 80% aqueous acetic acid and the solid was filtered. The solid was rinsed with acetic acid until the filtrate was clear. The solid was dried in a vacuum oven at 45° C. overnight to give a light tan powder that easily lifts off filter paper. The crude solid 4 has limited solubility in most solvents and thus was carried on without characterization.

### Example 5

### Synthesis of 3-amino-7-bromo-4-hydroxyquinoline (5)

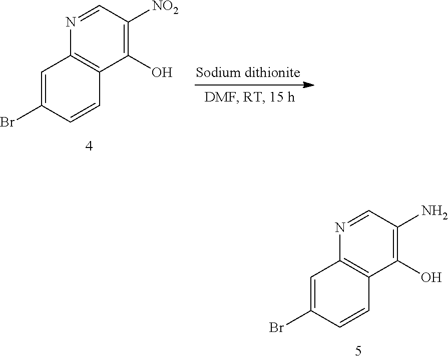 [SMILES: O=[N+]([O-])c1cnc2cc(Br)ccc2c1O ; Nc1cnc2cc(Br)ccc2c1O]

To a solution of crude 7-bromo-3-nitroquinolin-4-ol (0.34 g, 1.26 mmol) in anhydrous DMF (16 mL) was added sodium dithionite (1.0 g). The reaction mixture was stirred for 18 hours under N₂ at 25° C. DMF was removed en vacuo and the crude solid was suspended in ethyl acetate (15 mL) and then washed with hot water (35 mL). This mixture was stirred and heated while saturated aqueous sodium carbonate (10 mL) until all the remaining sodium dithionite had decomposed. The organics were separated from the aqueous layer and extracted with ethyl acetate (4×10 mL). Organics were combined and concentrated en vacuo. The crude compound was purified by silica column 85:9:3:3 EtOAc:MeOH:H₂O:Et₃N to give an off-white solid in 24.8% yield: ¹H NMR ((CD₃)₂SO, 400 MHz) δ 11.48 (br s, 1H), 8.01 (d, J=8.8 Hz, 1H), 7.68 (s, 1H), 7.55 (s, 1H), 7.29 (d, J=8.8 Hz, 1H), 4.72 (br s, 2H).

### Example 6

### Synthesis of 7-bromo-2-butylthiazolo[4,5-c]quinoline (6)

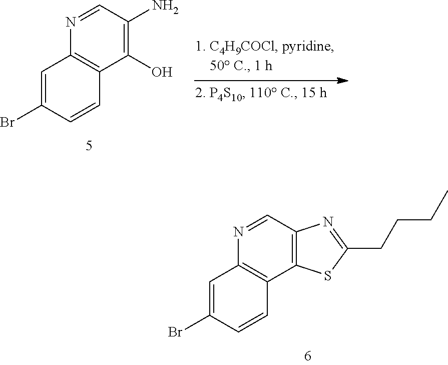 [SMILES: Nc1cnc2cc(Br)ccc2c1O ; CCCCc1nc2cnc3cc(Br)ccc3c2s1]

To a solution of 3-amino-7-bromo-4-hydroxyquinoline (0.07 g, 0.29 mmol, 1 equiv) in anhydrous pyridine (3.4 mL) being purged with N₂ was added valeryl chloride (0.044 g, 0.37 mmol, 1.25 equiv) and heated to 60° C. for 2 hours in a pressure vessel. The reaction was cooled to 25° C. and put under N₂ stream again. Phosphorus pentasulfide (0.14 g, 0.32 mmol, 1.1 equiv) was added and the reaction was heated to 105° C. for 18 hours in the pressure vessel. The reaction was cooled to 25° C. and aqueous sodium carbonate (10 mL) and ethyl acetate (10 mL) was added. The mixture was separated and the aqueous layer extracted with ethyl acetate (2×5 mL) (Caution, there will be trace hydrogen sulfide in the organics, usually pulling a light vacuum with some gentle heating for 2 hours will remove it). The organics were combined and concentrated en vacuo and purified by silica column 1:99 MeOH:DCM to give a yellow waxy solid in 86.1% yield: ¹H NMR (CDCl₃, 400 MHz) δ 9.43 (s, 1H), 8.43 (d, J=1.6 Hz, 1H), 7.84 (d, J=8.4 Hz, 1H), 7.73 (dd, J=8.4, 1.6 Hz, 1H), 3.23 (t, J=7.6 Hz, 2H), 1.94 (quint, J=7.6 Hz, 2H), 1.51 (app-sext, J=7.6, 7.2 Hz, 2H), 1.01 (t, J=7.2 Hz, 3H).

### Example 7

### Synthesis of 7-bromo-2-butylthiazolo[4,5-c]quinoline 5-oxide (7)

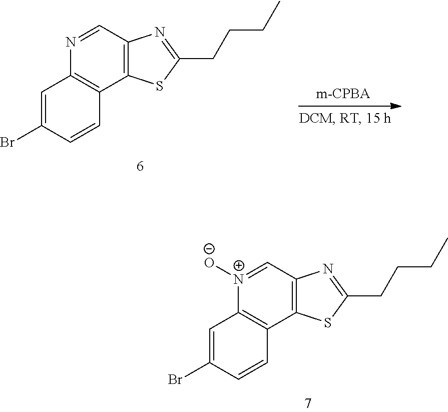 [SMILES: CCCCc1nc2cnc3cc(Br)ccc3c2s1 ; CCCCc1nc2c[n+]([O-])c3cc(Br)ccc3c2s1]

To a solution of 7-bromo-2-butylthiazolo[4,5-c]quinoline (0.075 g, 0.23 mmol, 1 equiv) in dichloromethane (3 mL) was added 53% m-chloroperoxybenzoic acid (0.27 g, 0.82 mmol, 3.5 equiv). This was stirred at 25° C. for 15 hours. TLC indicated a near complete reaction. The reaction mixture was washed with saturated aqueous sodium carbonate (10 mL), diluted with an additional portion of dichloromethane (10 mL) and water (10 mL), the mixture was separated, the aqueous layer was extracted with DCM (2×10 mL), and organics combined and concentrated en vacuo. The crude solid was purified by silica column 1:99-2:98 MeOH:DCM (linear gradient) to give a white powder in 92.7% yield: ¹H NMR (CDCl₃, 400 MHz) δ 9.19 (s, 1H), 9.11 (s, 1H), 7.84 (s, 2H), 3.20 (t, J=7.6 Hz, 2H), 1.92 (quint, J=7.6 Hz, 2H), 1.51 (sext, J=7.6, 2H), 1.00 (t, J=7.6 Hz, 3H).

### Example 8

### Synthesis of N-(7-bromo-2-butylthiazolo[4,5-c]quinolin-4-yl)benzamide (8)

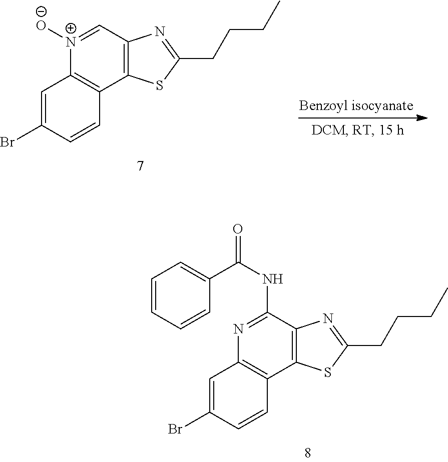 [SMILES: CCCCc1nc2c[n+]([O-])c3cc(Br)ccc3c2s1 ; CCCCc1nc2c(NC(=O)c3ccccc3)nc3cc(Br)ccc3c2s1]

To a solution of 7-bromo-2-butylthiazolo[4,5-c]quinoline 5-oxide (0.073 g, 0.22 mmol, 1 equiv) in dichloromethane (8 mL) was added benzoyl isocyanate (0.127 g, 0.86 mmol, 4 equiv). The solution turned a light yellow color after the benzoyl isocyanate was fully dissolved and was stirred at 25° C. for 18 hours. The reaction mixture was concentrated en vacuo and purified by silica column 0:100-1:99 MeOH:DCM (linear gradient) to give a white solid in 73.4% yield: ¹H NMR (CDCl₃, 400 MHz) δ 9.69 (br s, 1H), 8.41 (s, 1H), 8.08 (s, 1H), 8.06 (s, 1H), 7.70 (d, J=8.4 Hz, 1H), 7.65-7.53 (m, 4H), 3.21 (t, J=7.6 Hz, 2H), 1.92 (quint, J=7.6 Hz, 2H), 1.53 (app-sext, J=7.6, 7.2 Hz, 2H), 1.02 (t, J=7.2 Hz, 3H). ¹³C{¹H} NMR (CDCl₃, 600 MHz) δ 173.0, 164.6, 145.4, 144.3, 140.7, 139.3, 134.7, 132.4, 132.2, 129.2, 128.9, 127.7, 125.5, 122.9, 120.2, 34.0, 31.9, 22.3, 13.8. (ESI+): calcd C₂₁H₁₈BrN₃OS [M+H]+ 440.0427, found 440.0437 (error 2.3 ppm).

### Example 9

### Synthesis of N-(2-butyl-7-phenoxycarbonylthiazolo[4,5-c]quinolin-4-yl)benzamide (9)

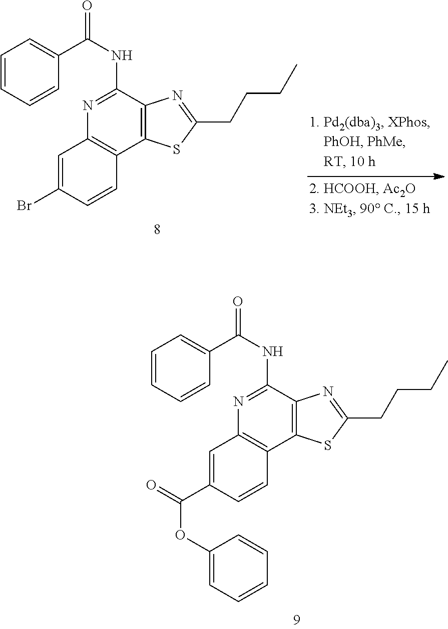 [SMILES: CCCCc1nc2c(NC(=O)c3ccccc3)nc3cc(Br)ccc3c2s1 ; CCCCc1nc2c(NC(=O)c3ccccc3)nc3cc(C(=O)Oc4ccccc4)ccc3c2s1]

A solution of formic acid (2 mL) in acetic anhydride (5 mL) was stirred at 35° C. for 1 hour to generate acetic formic anhydride. N-(7-bromo-2-butylthiazolo[4,5-c]quinolin-4-yl)benzamide (0.07 g, 0.16 mmol, 1 equiv), phenol (0.061 g, 0.65 mmol, 4.1 equiv), tris(dibenzylideneacetone)dipalladium(0) [Pd₂(dba)₃] (0.015 g, 0.016 mmol, 10 mol %), and 9,9-dimethyl-9H-xanthene-4,5-diylbis(diphenylphosphane) [XPhos] (0.021 g, 0.037 mmol, 23 mol %) were added to a flask and suspended in toluene (4 mL) under a stream of N₂. This mixture was stirred for 1 minute and then the premade acetic formic anhydride (75 μL, 0.95 mmol, 6 equiv) was added in 1 portion followed immediately by triethylamine (100 μL, 0.72 mmol, 4.5 equiv) while stirring. The flask was capped with a septum and stirred for 2 minutes. Then an additional portion of triethylamine was added (100 μL) (1.44 mmol, 9 equiv total) followed by an additional portion of acetic formic anhydride (30 μL) (1.33 mmol, 8.3 equiv total). The reaction was stirred and heated to 85° C. for 18 hours then cooled to 25° C. and diluted with ethyl acetate (5 mL). The crude reaction mixture was filtered through a small silica plug, concentrated en vacuo, and purified by silica column 1:99 MeOH:DCM-4:96 MeOH:DCM to give an off-white solid in 15.7% yield: ¹H NMR (CDCl₃, 600 MHz) δ 9.76 (br s, 1H), 9.17 (s, 1H), 8.25 (dd, J=8.4, 1.8 Hz, 1H), 8.09 (d, J=7.8 Hz, 2H), 7.94 (d, J=8.4 Hz, 1H), 7.65-7.43 (m, 5H), 7.35-7.75 (m, 3H), 3.25 (t, J=7.8 Hz, 2H), 1.95 (quint, J=7.8 Hz, 2H), 1.54 (app-sext, J=7.8, 7.2 Hz, 2H), 1.03 (t, J=7.2 Hz, 3H). ¹³C{¹H} NMR (CDCl₃, 600 MHz) δ 174.4, 164.7, 164.4, 151.0, 145.4, 143.1, 140.3, 140.2, 134.6, 132.4, 129.9, 129.6, 129.51, 129.50, 128.9, 127.6, 126.1, 125.9, 124.8, 121.6, 34.0, 31.9, 22.3, 13.7. (ESI+): calcd C₂₈H₂₃N₃O₃S [M+H]+ 482.1533, found 482.1572 (error 8.1 ppm).

### Example 10

### Synthesis of 4-amino-2-butyl-7-methoxycarbonylthiazolo[4,5-c]quinoline (1)

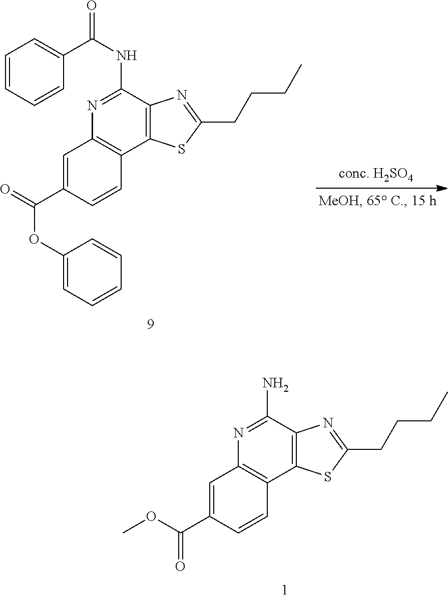

N-(2-Butyl-7-phenoxycarbonylthiazolo[4,5-c]quinolin-4-yl)benzamide (0.010 g, 0.020 mmol) was added to a small vial and suspended in anhydrous methanol (3 mL). The mixture was sonicated for 1 minute to break up any larger solid chunks. Under a stream of N₂, concentrated sulfuric acid (3 drips, approximately 125 mg) was added which quickly dissolved the fine suspended solids. The vial was capped and stirred and heated at 60° C. for 15 hours. The reaction was cooled to 25° C. and neutralized with solid sodium carbonate powder, diluted with ethyl acetate (5 mL) and water (5 mL), and stirred for 10 minutes. This mixture was concentrated en vacuo and redissolved in the same amount of ethyl acetate and water. The mixture was separated, and the aqueous was extracted with ethyl acetate (3×5 mL). The organics were combined and concentrated en vacuo. The crude was purified by silica column 25:75 EtOAc:Hexs to give the final product as a white solid in 68.7% yield: ¹H NMR (CDCl₃, 600 MHz) δ 8.53 (d, J=1.2 Hz, 1H), 8.03 (dd, J=8.4, 1.2 Hz, 1H), 7.81 (d, J=8.4 Hz, 1H), 6.86 (br s, 2H), 3.98 (s, 3H), 3.20 (t, J=7.8 Hz, 2H), 1.91 (app-quint, J=7.8, 7.2 Hz, 2H), 1.51 (sext, J=7.2 Hz, 2H), 1.01 (t, J=7.2 Hz, 3H). ¹³C{¹H} NMR (CDCl₃, 600 MHz) δ 174.5, 166.2, 151.0, 141.5, 138.7, 138.1, 131.5, 125.5, 125.2, 124.6, 121.5, 52.6, 33.9, 31.8, 22.2, 13.7. (APCI+): calcd C₁₆H₁₇N₃O₂S [M+H]+ 316.1114, found 316.1108 (error 1.9 ppm).

This synthetic route provides a superior route allowing more diverse substitutions to the C7 positions of 4-amino-quinolines and demonstrated by synthesizing 4-amino-2-butyl-7-methoxycarbonylthiazolo[4,5-c]quinoline 1. The approach overcomes the limitation of conventional routes requiring formation of a 5N-oxide intermediate that is not compatible with electron withdrawing groups at the C7 position. Instead, it was surprisingly found that d catalyzed carbonylation reactions allow the design and efficient and scalable synthesis of substituted quinoline nucleoside base analogs as well as highly substituted isoquinolines.

### Example 11

### Synthesis of 4-amino-2-butyl-7-methoxycarbonyl thiazolo[4,5-c]quinoline (1)

Compound 1 was synthesized according to the following reaction conditions.

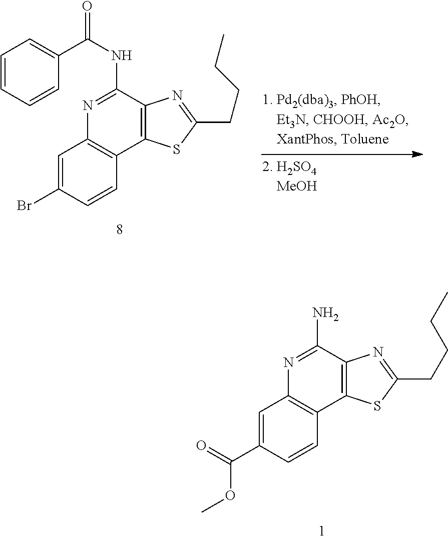 [SMILES: CCCCc1nc2c(NC(=O)c3ccccc3)nc3cc(Br)ccc3c2s1 ; CCCCc1nc2c(N)nc3cc(C(=O)OC)ccc3c2s1]

### Example 12

### Heyerocyclic Compounds of the Present Disclosure

The compounds of TABLE 2 were produced using the synthetic methods described in EXAMPLES 1-11 and reaction SCHEMES 1-5.

TABLE 2
Compounds produced according to embodiments of the disclosure

| Cmpd | | |
|---|---|---|
| No. | Structure | Characterization |
| 1 | 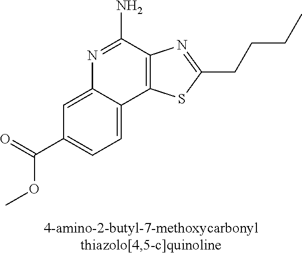 [SMILES: CCCCc1nc2c(N)nc3cc(C(=O)OC)ccc3c2s1] | ¹H NMR (CDCl₃, 600 MHz) δ 8.53 (d, J = 1.2, 1H), 8.03 (dd, J = 8.4, 1.2 Hz, 1H), 7.81 (d, J = 8.4, 1H), 6.86 (br s, 2H), 3.98 (s, 3H), 3.20 (t, J = 7.8 Hz, 2H), 1.91 (app- quint, J = 7.8, 7.2 Hz, 2H), 1.51 (sext, J = 7.2, 7.2 Hz, 2H), 1.01 (t, J = 7.2 Hz, 3H). ¹³C{¹H} NMR (CDCl₃, 600 MHz) δ 174.5, 166.2, 151.0, 141.5, 138.7, 138.1, 131.5, 125.5, 125.2, 124.6, 121.5, 52.6, 33.9, 31.8, 22.2, 13.7. (APCI+): calcd C₁₆H₁₇N₃O₂S [M + H]+ 316.1114, found 316.1108. |
| 22 | 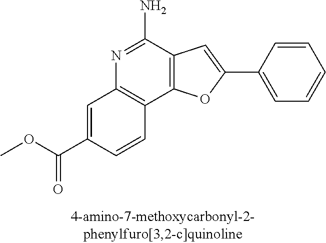 [SMILES: COC(=O)c1ccc2c(c1)nc(N)c1cc(-c3ccccc3)oc12] | ¹H NMR (CDCl₃, 400 MHz) δ 8.43 (s, 1H), 8.14 (d, J = 8.4 Hz, 1H), 7.99 (d, J = 8.4 Hz, 1H), 7.87 (d, J = 8.0 Hz, 2H), 7.52-7.38 (m, 3H), 7.21 (s, 1H), 6.26 (br s, 2H), 3.94 (s, 3H). (MM+): calcd C₁₉H₁₄N₂O₃ [M + H]+ 319.1077, found 319.1070. |
| 23 | 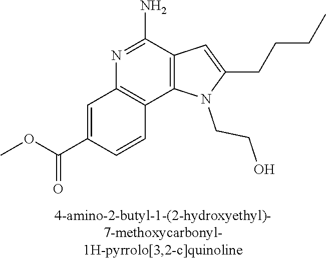 [SMILES: CCCCc1cc2c(N)nc3cc(C(=O)OC)ccc3c2n1CCO] | ¹H NMR (DMSO-d₆, 400 MHz) δ 8.15 (d, J = 8.8, 1H), 8.14 (d, J = 2.0 Hz, 1H), 7.68 (dd, J = 8.8, 2.0 Hz, 1H), 6.68 (s, 1H), 6.61 (s, 2H), 5.07 (t, J = 4.8 Hz, 1H), 4.54 (t, J = 6.0 Hz, 2H), 3.88 (s, 3H), 3.79 (dd, J = 6.0, 4.8 Hz, 2H), 2.83 (t, J = 7.6 Hz, 2H), 1.72 (quint, J = 7.6 Hz, 2H), 1.48 (app-sext, J = 7.6, 7.2 Hz, 2H), 0.98 (t, J = 7.2 Hz, 3H). (APCI+): calcd C₁₉H₂₃N₃O₃ [M + H]+ 342.1812, found 342.1806. |
| 24 | 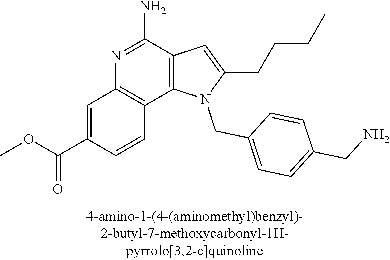 [SMILES: CCCCc1cc2c(N)nc3cc(C(=O)OC)ccc3c2n1Cc1ccc(CN)cc1] | ¹H NMR (DMSO-d₆, 400 MHz) δ 8.05 (s, 1H), 7.87 (d, J = 9.0 Hz, 1H), 7.43 (d, J = 9.0 Hz, 1H), 7.20 (d, J = 8.0 Hz, 2H), 6.84 (d, J = 8.0 Hz, 2H), 6.79 (s, 1H), 6.68 (s, 2H), 5.74 (s, 2H), 3.79 (s, 3H), 3.59 (s, 2H), 2.71 (t, J = 7.6 Hz, 2H), 1.62 (quint, J = 7.6 Hz, 2H), 1.37 (app-sext, J = 7.6, 7.2 Hz, 2H), 0.87 (t, J = 7.2 Hz, 3H). (APCI+): calcd C₂₅H₂₈N₄O₂ [M + H]+ 417.2285, found 417.2284. |
| 25 | 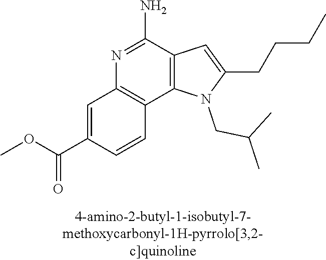 [SMILES: CCCCc1cc2c(N)nc3cc(C(=O)OC)ccc3c2n1CC(C)C] | ¹H NMR (CDCl₃, 400 MHz) δ 8.51 (d, J = 1.8, 1H), 8.01 (d, J = 8.4 Hz, 1H), 7.94 (dd, J = 8.4, 1.8 Hz, 1H), 6.36 (s, 1H), 5.13 (br s, 2H), 4.25 (br s, 2H), 3.96 (s, 3H), 2.78 (t, J = 7.6 Hz, 2H), 2.34 (app-sept, J = 7.2, 6.8 Hz, 1H) 1.77 (quint, J = 7.6 Hz, 2H), 1.50 (app- sext, J = 7.6, 7.2 Hz, 2H), 1.01 (t, J = 7.2 Hz, 3H), 0.96 (t, J = 6.4 Hz, 6H). (MM+): calcd C₂₁H₂₇N₃O₂ [M + H]+ 354.2176, found 354.2182. |
| 26 | 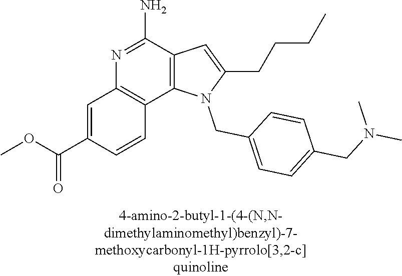 | ¹H NMR (DMSO-d₆, 400 MHz) δ 8.09 (s, 1H), 7.90 (d, J = 8.4 Hz, 1H), 7.46 (d, J = 8.4 Hz, 1H), 7.20 (d, J = 8.0 Hz, 2H), 6.91 (d, J = 8.0 Hz, 2H), 6.82 (s, 1H), 6.71 (s, 2H), 5.78 (s, 2H), 3.83 (s, 3H), 3.29 (s, 2H), 2.75 (t, J = 7.6 Hz, 2H), 2.06 (s, 6H) 1.63 (app-quint, J = 7.6, 7.2 Hz, 2H), 1.38 (sext, J = 7.2 Hz, 2H), 0.87 (t, J = 7.2 Hz, 3H). (APCI+): calcd C₂₇H₃₂N₄O₂ [M + H]+ 445.2598, found 445.2597. |

### Example 13

### Testing of Compounds Using a TLR7/8-NF-κB Reporter Assay

Human embryonic kidney (HEK) cells that were stably transfected with human TLR-7 or TLR-8 and an NF-κB-responsive secreted embryonic alkaline phosphatase (SEAP) gene (HEK-TLR-7 and -8) are purchased from InvivoGen (San Diego, CA). HEK-TLR7/8 cells are stimulated with 30 μM of compound in a 96-well plate in DMEM containing 10% FBS and 0.01% Normocin (InvivoGen) for 24 h. 20 μL of the supernatant from each well is incubated with Quanti-blue substrate solution (InvivoGen) at 37° C. for 1 h and absorbance is read at 650 nm using a Synergy plate reader (Biotek, Winooski, VT).

### Example 14

### Measurement of Proimflammatory Cytokines with Cytometric Bead Assay

Bone marrow derived dendritic cells (BMDC) are generated by isolating a single cell suspension of marrow from the femur of C57BL/6 mice (6-8 weeks of age). Red blood cells are lysed with 0.83% NH₄Cl, 0.1% KHCO₃ and 0.009%. 5 million cells are seeded in each of well of a 6 well plate in complete RPMI media (Invitrogen, Grand Island, NY), supplemented with mouse 20 ng/ml Granulocyte-Macrophage Colony Stimulating Factor (PeproTech, Rocky Hill, NJ). About 6 days after culture, BMDC are stimulated with 30 μM of compound for 3 days. 25 μL of supernatant is then removed and assayed for TNFα, IL-12p40, IL-1β and IL-10 using a flow cytometric bead array according to the manufacturers' instructions (BD Bioscience, San Jose, CA). Controls are performed using the addition of media and carrier with no drug. Flow cytometry is performed on a FACS canto-II (BD Bioscience) and data are analyzed using Flowjo software (Tree Star, Inc. Ashland, OR).

### Example 15

### Measurement of Proimflammatory Cytokines with Cytometric Bead Assay

Dendritic cells are generated from peripheral blood monocytes as described (Brossart P, et al. Blood. 1998; 92: 4238-4247). In brief, CD14 positive monocytes are from a healthy human peripheral blood mononuclear cells (PBMC) obtained via isolation with Lymphocyte Separation Medium (Mediatech, Inc, Manassas, VA) and after purification with CD14 microbeads from Miltenyi Biotec Inc (Auburn, CA). The CD14 positive monocytes (>95% CD14) are cultured into immature monocyte-derived dendritic cells (MoDC) by further 6-day culture with GM-CSF (100 ng/ml) and IL-4 (100 ng/ml) (R&D, MN).

About 0.1 million of MoDC cells are plated into a 96-well plate and stimulated for 48 hours with 5 different concentration of the TLR-modulating compounds of the present disclosure at concentrations of 0, 0.325, 1.3, 5.2 and 20.8 nmol/ml in triplicate. For immunostaining and flowcytometric analysis, and about 48 hours after stimulation with the compounds, the cells are stained with anti-HLA-DR, CD11c, CD-86, CD80, CD83, CD8a, CD123 and relevant isotype controls (eBioscience, San Diego, CA). The cells are loaded on FACS-canto II and analyzed with FACSDiva and Flowjo. For Cytometric Bead Assay (CBA), the supernatant is harvested 48 hours after stimulation with the TLR modulating compounds. Inflammatory cytokines level is identified with CBA, following the producer's instruction (BD, San Jose, CA).

### Example 16

### Evaluation of IL-6 Levels after Stimulation with Compounds of the Disclosure

TLR7 mutant mice and C57BL/6j mice, 8-12 weeks old, are obtained from Jackson Lab (Bar Harbor, Maine). TLR7 mutant gene is introduced to 129S1/Sv derived from CJ7 embryonic stem cells. The cell line is backcrossed ten times to C57BL/6Ncr. No TLR7 RNA expression is detected in bone marrow-derived macrophages. The homologues TLR7 mutant mice are developed from backcrossing heterologous mutant mice with wild type C57BL/6j.

Single cell suspensions of splenocytes from C57BL/6j or TLR7 mutant mice is isolated after whole spleen is squeezed through 70 μm cell strainer and red blood cell lysis process. Splenocytes are pulsed in triplicate with 2.08 nmol/ml or 20.8 nmol/ml of Imiquimod (IMQ), hydroxyl Imiquimod (IMQ-OH) or 10 μg/ml of CpG685 in complete RPMI-1640 medium (10% heat-inactivated FBS, glutamine, 1% penicillin/streptomycin, 55 nmol 2-ME, 10 mmol HEPES). Supernatant from the culture medium is harvested 12 hours and 24 hours after pulsing and frozen at −80° C. until analysis. A cytometric bead array (BD Biosciences, San Jose, CA) are used for measurement of IL-6 level according to the manufacture's instruction. An analysis is performed on FACScanto-II machine with FACSAria II software and further analyzed with Flowjo software (Tree Star, Inc, Ashland, OR). Standard curves and negative control (PBS) are included for calculation of the cytokine concentration in the samples.

### Example 17

### In Vitro Binding Ability Screening to TLR7/8-Expressing Cells

TLR-7 or TLR-8 positive cell lines, e.g., HEK-Blue TLR cells (Invivogen, San Diego, CA), are used for this screening assay. HEK-Blue TLR cells are engineered HEK293 cells. Such cells stably express TLR gene and an inducible NF-kB-SEAP (secreted embryonic alkaline phosphase) reporter gene. Bounding of ligands, e.g., compounds of this disclosure, with TLR in HEK-Blue cells induces SEAP to generate blue color. Screening assays are conducted following the manufacture's instruction. TLR targeting compounds, at 20.8 nmol/ml or 5.2 nmol/ml concentration, are added in triplicate to HEK-Blue-TLR7 or TLR8 cells, cultured at 37° C. and 5% CO₂ condition. 24 hours later, 5 μl of supernatant of cultures is mixed with 200 μl of pNPP-included detection medium. After one hour, SEAP activity is read out as OD at 650 nm with a microplate reader (BioTek Synergy 2, Vermont).

### Example 18

### Inflammatory Cytokine Detection in BMDC and Splenocytes

Bone marrow cells are harvested from femurs and tibias of C57BL/6j mice. After red blood cells are removed with ammonium-chloride-potassium buffer, the bone marrow cells are cultured with complete RPMI-1640 medium and 2 ng/ml of granulocyte macrophage colony-stimulating factor (GM-CFS) at 5% CO₂ and 37° C. for 6 days. Medium is changed twice during the 6 days culture. Single cell suspension of splenocytes is prepared as described herein. BMDC or splenocytes are stimulated in triplicate with test compounds of Formula I at various concentrations of 20.8 nmol/ml, 5.2 nmol/ml, 1.3 nmol/ml, 0.325 nmol/ml and 0 nmol/ml. 48 hours after stimulation, the supernatants are harvested and frozen at −80° C. until analysis. A cytometric bead array (CBA, BD Bioscience) is performed on inflammatory cytokines following the manufacture's instruction. 500 events are collected. Analysis of all samples is performed on FACScanto-II machine with software and further analyzed with Flowjo. Standard curves and negative control (PBS) are included for each cytokine to calculate the cytokine concentration in the samples.

Detection of co-stimulator level on BMDC. Two days after stimulation with test compounds of Formula I, the BMDCs are stained in triplicate with different fluorocore-labeled antibodies obtained from eBioscience (San Diego, CA). The antibodies include anti-MHC-II (I-A/I-E, clone M5/114.15.2), anti-CD86 (clone GL1), anti-CD80 (clone 16-10A1), anti-CD8α (clone 53-6.7), anti-CD11b (clone, M1/70), antiCD-205 (clone 205yekta), anti-CD3 (clone 17A2) and anti-CD11c (clone N418). All samples are acquired on a FACSCanto II flow cytometer (BD Biosciences, San Jose, CA). Between 50,000 and 100,000 events are collected. All data are analyzed with Flowjo software (Tree Star, Inc, Ashland, OR). Gate is based on CD3-CD11c+ population.

Proliferation of pmel CD8 and IFNγ production after cross-presentation. BMDCs from C56BL/6j mice and single cell suspension of splenocytes from pmel mice (T-cell receptor transgenic mice containing human gp100₂₅₋₃₃\H2db specific receptors, Jackson Lab) are prepared as described above in this EXAMPLE. BMDCs are pulsed in triplicate with 3.5 μg of human gp100 peptide per well (Peptide sequence: CALLAVGATKVPRNQDWLGVSRQLRTK, GenScript, Piscataway, NJ) and test compound of Formula I at the concentration of 10.4 nmol/ml and hgp100 peptide control and PBS negative control for 48 hours. BMDCs are washed twice with complete RPMI medium followed by coculture with pmel CD8 splenocytes that are CFSE-labeled and that are isolated from pmel splenocytes with CD8+T Cell isolation Kit (Miltenyi Biotec, Auburn, CA) at a ratio of 1:3 of DC/CD8. Four days after coculture, supernatants are harvested and frozen at −80° C. until detection of INFγ with CBA kit. CBA for IFNγ measurement is conducted according to manufacture's instruction. The cell pellets are washed and stained with fluorocore-labeled antibodies, all of which are obtained from eBioscience. They are anti-CD3 (clone, 17A2) and anti-CD8α (clone 53-6.7). Flowcytometric data are acquired from the stained samples on FACSCanto II floweytometer and analyzed with Flowjo software. Gate is from CD3+CD8+ populations.

IL-2 production of OT-I cells after stimulation with IMQ-derived new TLR7 ligands. Single bone marrow cell suspensions from C57BL/6j mice are prepared as previously described in this EXAMPLE. The cells are pulsed in triplicate with test compounds of Formula I at 20.8 nmol/ml concentration and added with and without 15 μg of ovalbumin (Sigma-Aldrich, St. Louis, MO) per well. Four days later, the cells are washed twice with complete RPMI medium and cultured with isolated OT-I CD8 T cells using CD8+isolation Kit, (Miltenyl Biotec, Auburn, CA). After four days of coculture, the supernatants are harvested and detected. CBA is conducted for IL-2 production according to BD Bioscience's instruction. Data are acquired on FACSCanto-II flowcytometer and 500 events are collected and analyzed with Flowjo software.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by some embodiments, exemplary embodiments and optional features, modification and variation of the concepts herein disclosed may be resorted to by those skilled in the art, and that such modifications and variations are considered to be within the scope of this disclosure as defined by the appended claims. The specific embodiments provided herein are examples of useful embodiments of the present disclosure and it will be apparent to one skilled in the art that the present disclosure may be carried out using a large number of variations of the devices, device components, methods steps set forth in the present description. As will be obvious to one of skill in the art, methods and devices useful for the present methods can include a large number of optional composition and processing elements and steps.

What is claimed is:

1. A compound of Formula I: 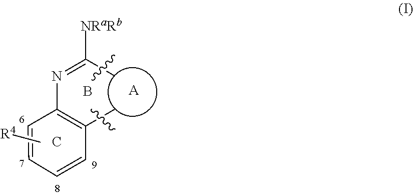
wherein:
the fused ring A is selected from the group consisting of: 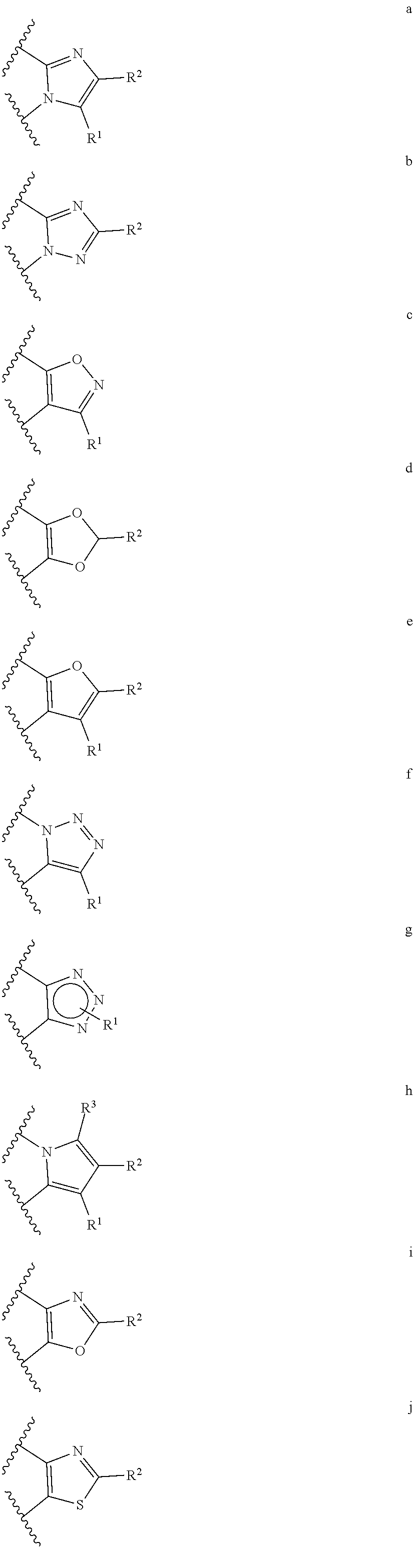 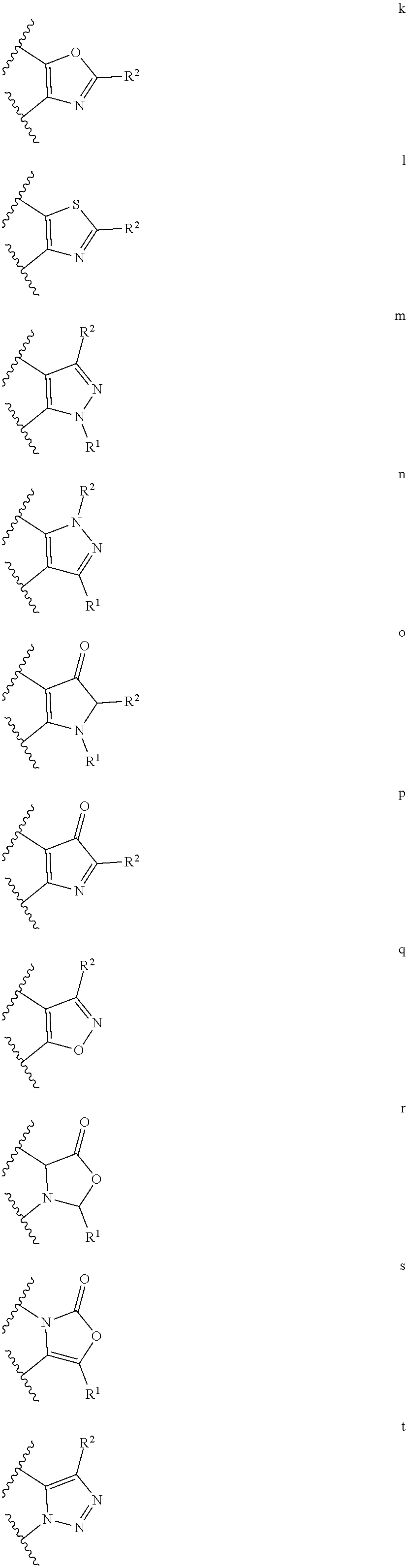 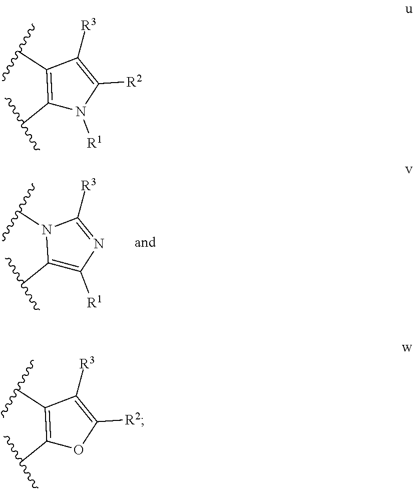
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{w}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

2. A compound of Formula I: 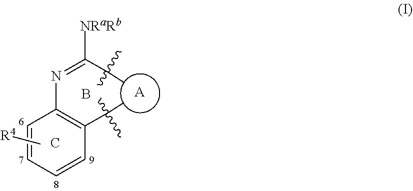
wherein:
the fused ring A is selected from the group consisting of: 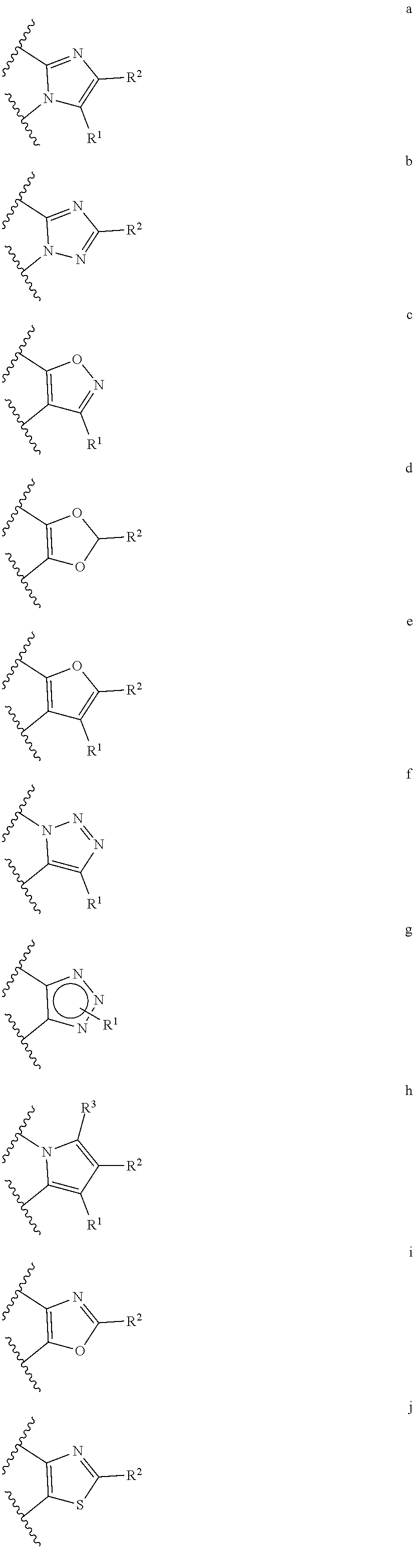 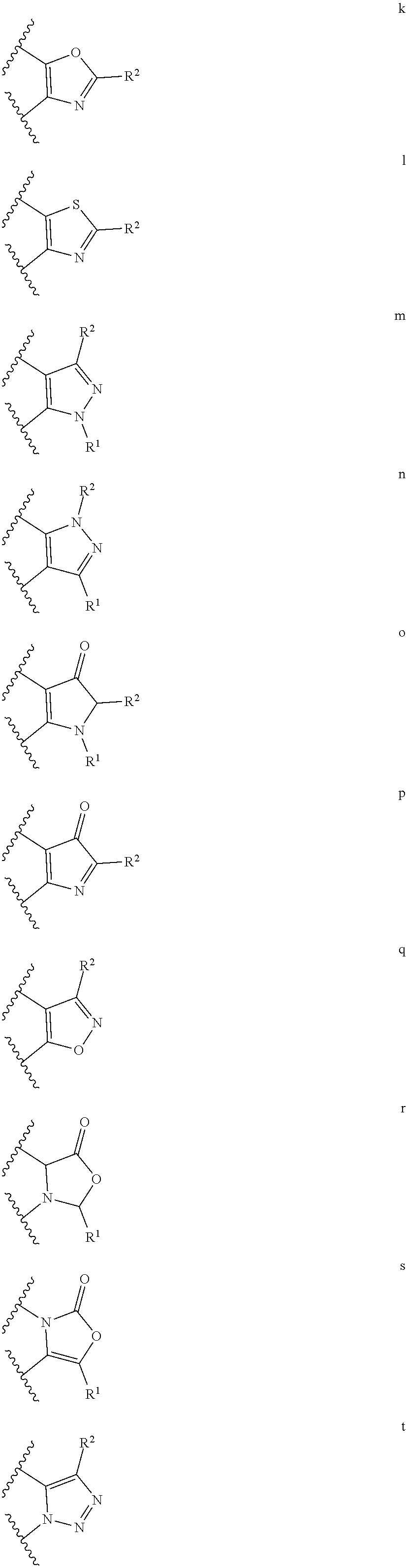 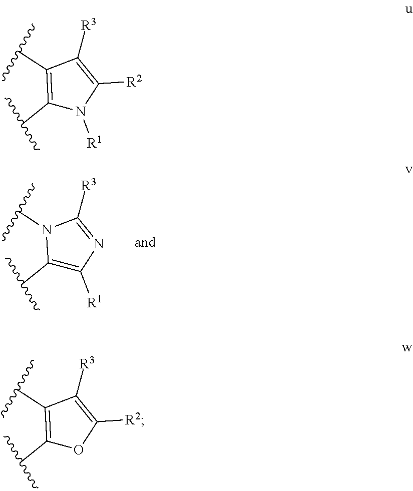
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

3. The compound of claim 1 or 2, wherein R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, or (C₃-C₈)cycloalkyl.

4. The compound of any one of claims 1-6, wherein ring A is selected from the group consisting of:

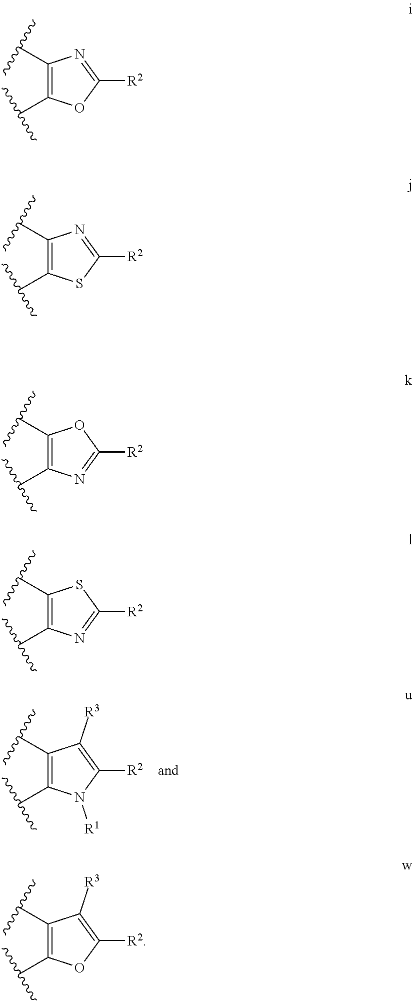

5. The compound of claim 4, wherein R¹ and R³ are each independently H or optionally substituted (C₁-C₆)alkyl.

6. The compound of claim 4 or 5, wherein R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

7. The compound of any one of claims 1-6, wherein ring A is selected from the group consisting of:

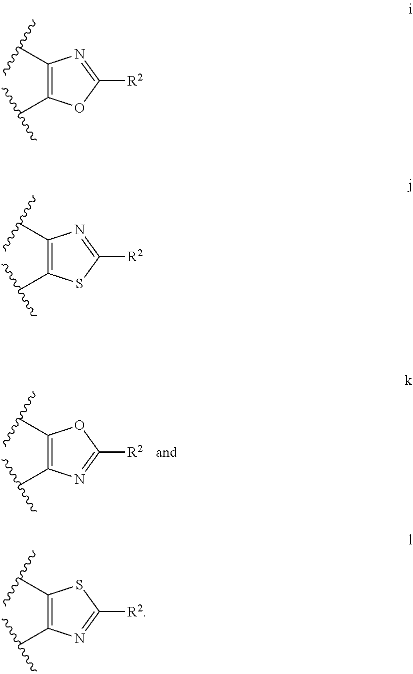

8. The compound of any one of claims 1-7, wherein R² is H or optionally substituted (C₁-C₆)alkyl.

9. The compound of any one of claims 6-8, wherein ring A is:

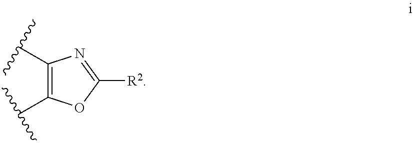

10. The compound of any one of claims 6-8, wherein ring A is:

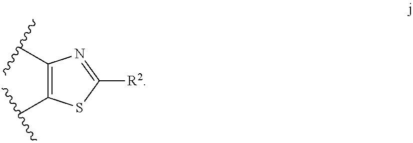

11. The compound of any one of claims 6-8, wherein ring A is:

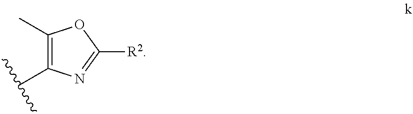

12. The compound of any one of claims 6-8, wherein ring A is:

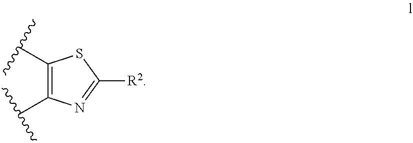

13. A compound of Formula I: 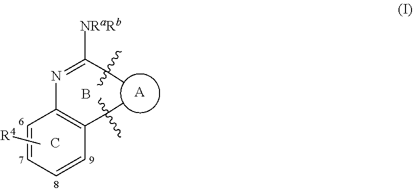
wherein:
the fused ring A is: 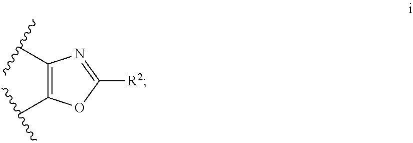
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

14. A compound of Formula I: 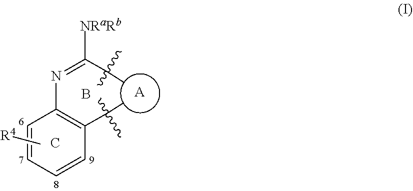
wherein:
the fused ring A is: 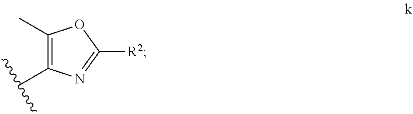
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

15. A compound of Formula I: 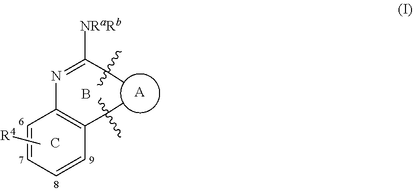
wherein:
the fused ring A is: 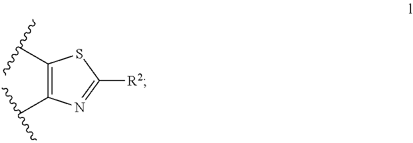
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h},
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

16. A compound of Formula I: 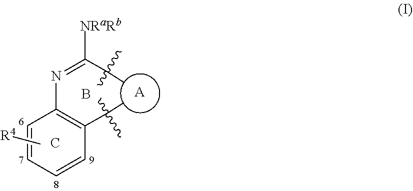
wherein:
the fused ring A is: 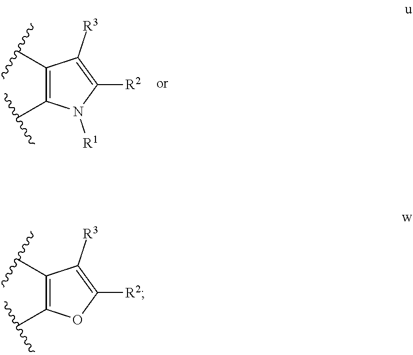
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}; and
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

17. A compound of Formula II: 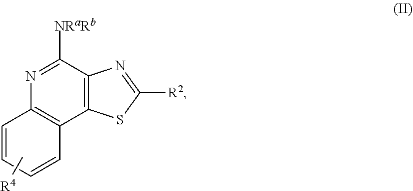
or a pharmaceutically acceptable salt thereof,
wherein:
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)— or R^{k}—O—C(═O)—;
R^{a} is H or optionally substituted (C₁-C₆)alkyl;
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

18. The compound of claim 17, wherein R² is H or optionally substituted (C₁-C₆)alkyl.

19. The compound of either claim 17 or 18, wherein R^{k} is H or optionally substituted (C₁-C₆)alkyl.

20. The compound of any one of claims 17-19, having the formula of:

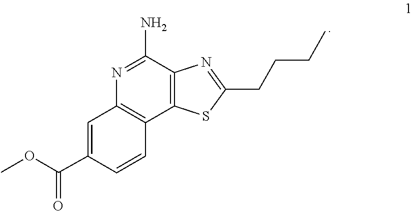 [SMILES: CCCCc1nc2c(N)nc3cc(C(=O)OC)ccc3c2s1]

21. A compound of Formula III or Formula IV: 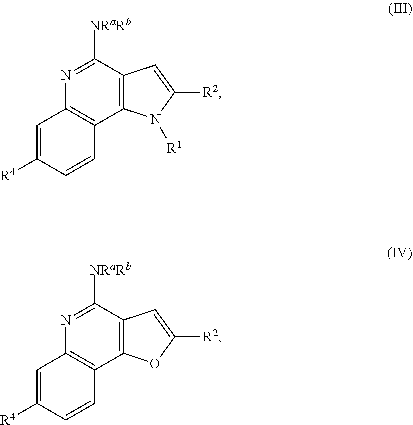
or a pharmaceutically acceptable salt thereof,
wherein:
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, or R^{k}—S(O)₂—O—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
X is a linking group; and
Y is a peptide, a protein, or maleimide.

22. The compound of claim 21, wherein R¹ and R² are independently H or (C₁-C₆)alkyl that can be optionally substituted with (C₁-C₃)alkyl, substituted or unsubstituted (C₃-C₈)cycloalkyl or substituted or unsubstituted aryl.

23. The compound of claim 21 or 22, wherein R⁴ is R^{k}—O—C(═O)—, or R^{k}—S(O)₂—O—.

24. The compound of claim 21 or 22, selected from the group consisting of:

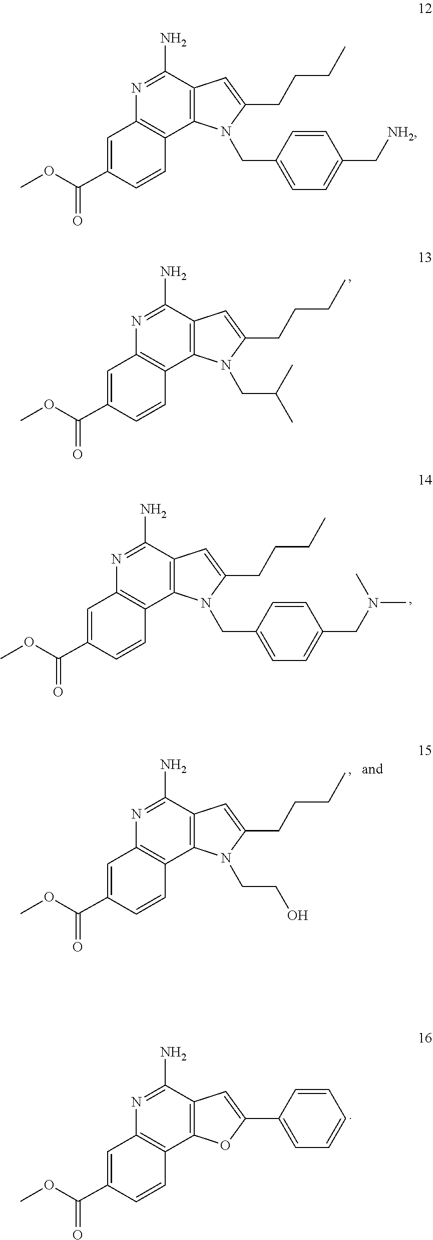

25. The compound of any one of claims 1-19 or 21-23, wherein X is (C₁-C₆)alkyl, (C₂-C₆)alkenyl, or (C₁-C₆)alkynyl, which (C₁-C₆)alkyl, (C₂-C₆)alkenyl, or (C₁-C₆)alkynyl is optionally substituted with oxo.

26. The compound of any one of claims 1-19 or 21-23, wherein X is selected from the group consisting of: 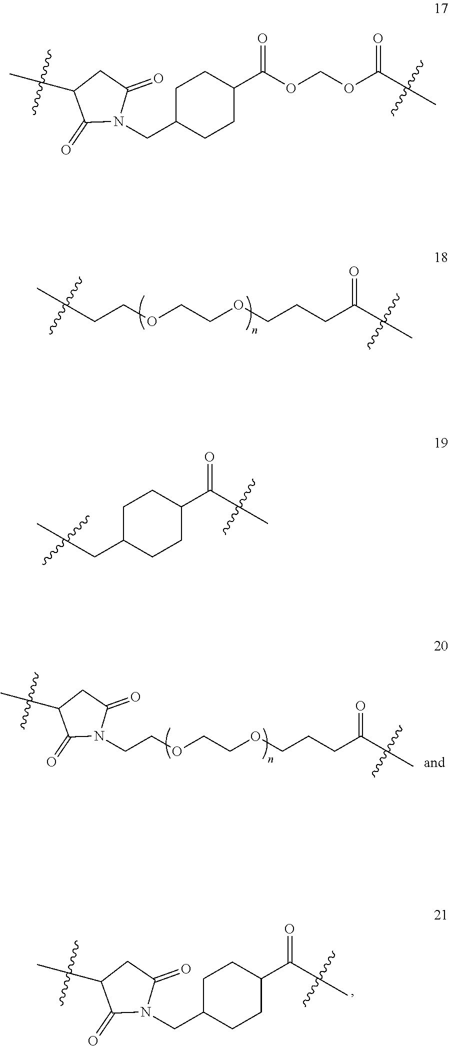
and wherein n is 2, 3, 4, 5, or 6.

27. The compound of any one of claims 1-19, 21-23, or 25-26, wherein Y is maleimide.

28. The compound of any one of claims 1-19, 21-23, or 25-26, wherein Y is an antigen.

29. The compound of claim 28, wherein the antigen is associated with a bacterium or a virus, and wherein the virus is selected from the group consisting of an influenza virus, HIV, and HCV.

30. The compound of any one of claims 1-19, 21-23, or 25-26, wherein Y is an antigen associated with a tumor cell or a tumor cell lysate.

31. The compound of any one of claims 28-30, wherein Y is an antigen comprising a peptide sequence comprising cysteine, lysine, or both.

32. The compound of any one of claims 1-31, wherein the compound is any one of compounds 1 or 22-26.

33. A compound of Formula I: 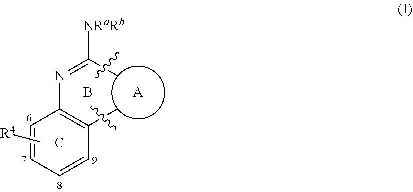
wherein:
the fused ring A is selected from the group consisting of: 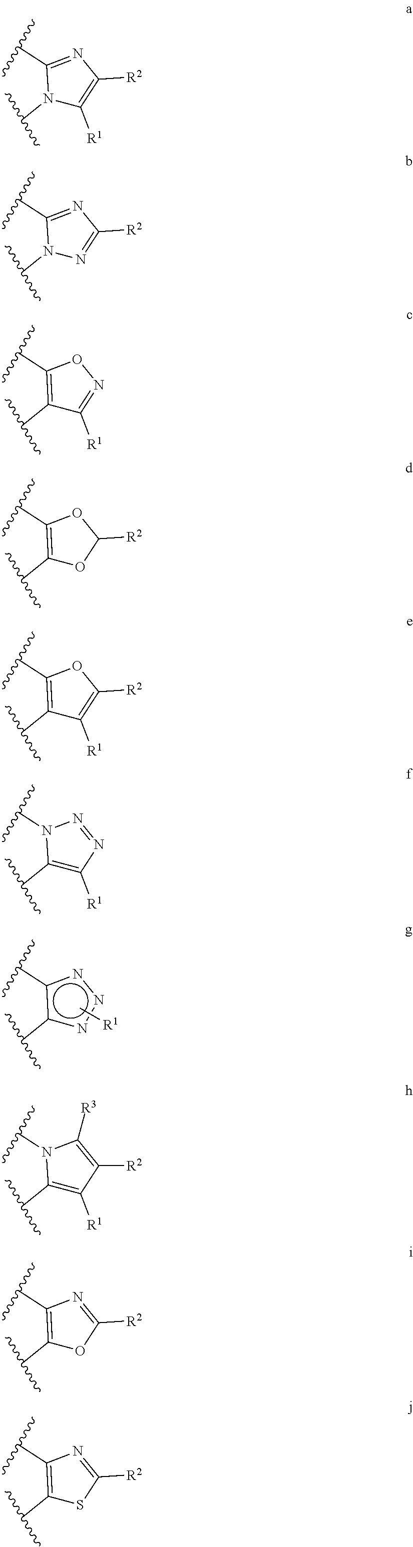 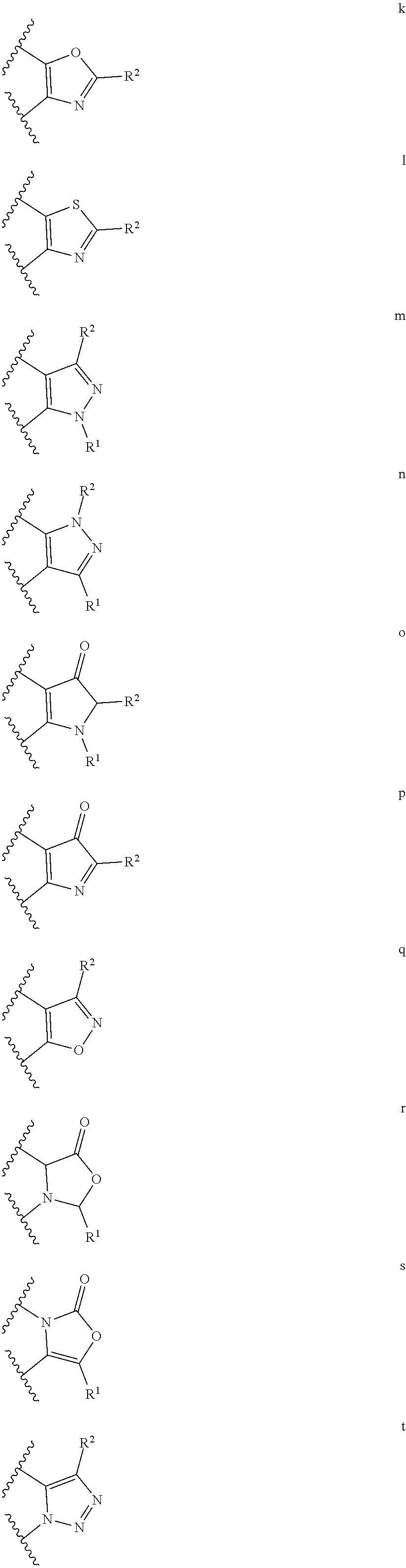 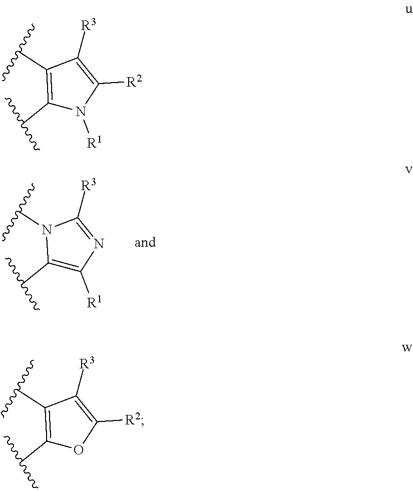
R¹ is H, (C₁-C₆)alkyl, (C₂-C₆)alkenyl, (C₂-C₆)alkynyl, (C₁-C₁)alkanoyl, (C₁-C₆)alkoxycarbonyl, (C₁-C₆)alkanoyloxy, (C₃-C₆)cycloalkyl, aryl, heteroaryl, heterocycle, NRR or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, or heteroaryl, wherein any (C₁-C₆)alkyl, (C₂-C₆)alkenyl, (C₂-C₆)alkynyl, (C₁-C₆)alkanoyl, (C₁-C₆)alkoxycarbonyl, (C₃-C₆)cycloalkyl, aryl, and heteroaryl is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

34. A compound of Formula I: 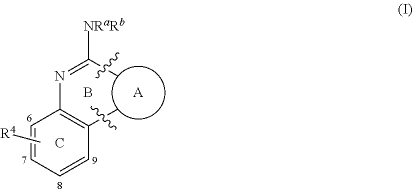
wherein:
the fused ring A is selected from the group consisting of: 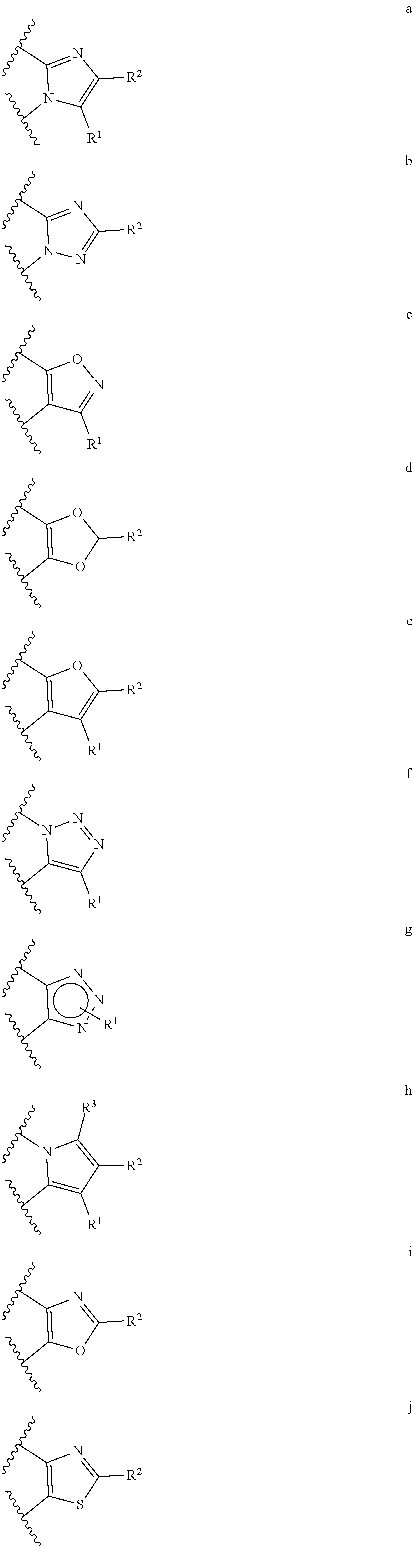 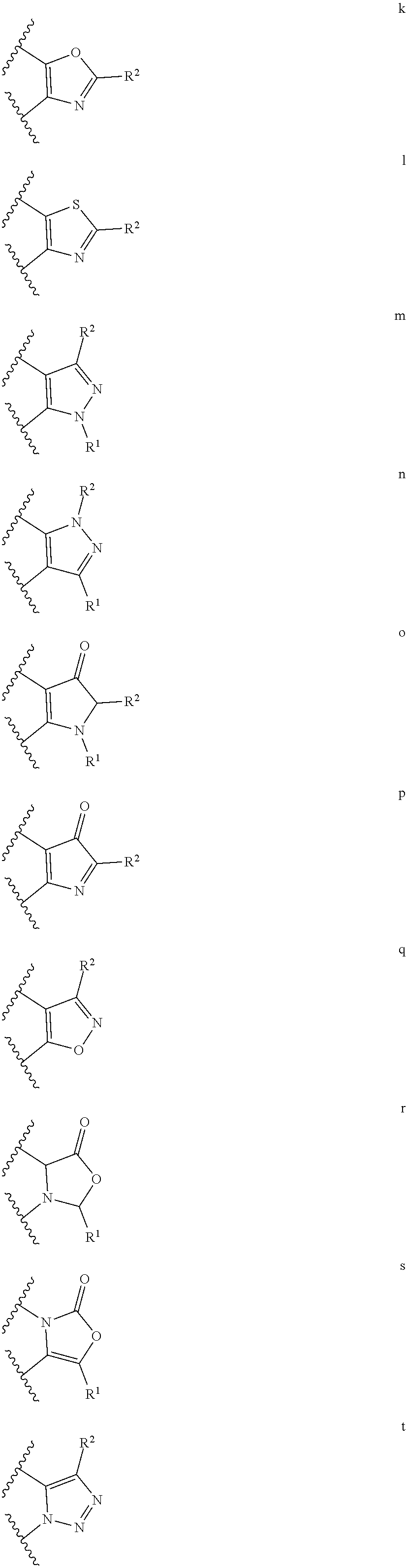 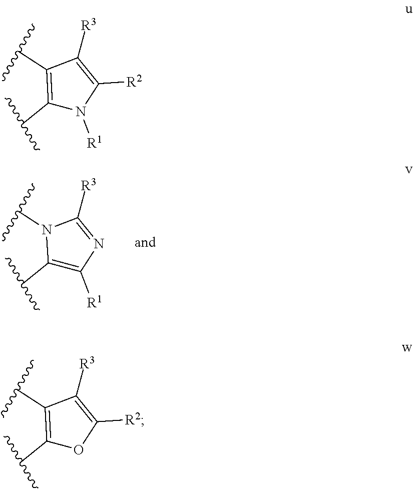
R¹ is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, and heterocycle, is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy;
R² is H, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, heterocycle, NR^{g}R^{h}, or R^{m}RⁿNC(═O)—, wherein any (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)cycloalkyl, aryl, heteroaryl, and heterocycle, is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h};
R⁴ is R^{k}—C(═O)—, R^{k}—O—C(═O)—, R^{k}—S(O)₂—O—, R^{c}R^{d}NC(═O)—, R^{c}R^{d}NS(O)₂—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NC(═O)—, R^{c}R^{d}NS(O)₂—, R^{e}C(═O)N(R^{e})—, or R^{e}S(O)₂NR^{f}—;
R^{a} is H, (C₁-C₆)alkyl, (C₂-C₆)alkenyl, (C₂-C₆)alkynyl, (C₁-C₆)alkanoyl, or (C₁-C₆)alkoxycarbonyl, wherein any (C₁-C₆)alkyl, (C₂-C₆)alkenyl, (C₂-C₆)alkynyl, (C₁-C₆)alkanoyl, and (C₁-C₆)alkoxycarbonyl, is optionally substituted with one or more groups independently selected from the group consisting of hydroxy, halo, —SH, cyano, oxiranyl, (C₃-C₈)cycloalkyl, aryl, heteroaryl, (C₁-C₆)alkoxy, (C₁-C₆)alkylthio, and NR^{g}R^{h};
R^{b} is H or X—Y;
each R^{c} and R^{d} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{e} is H, (C₁-C₆)alkyl, or R^{aa};
R^{f} is H or (C₁-C₆)alkyl;
each R^{g} and R^{h} is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{k} is H, (C₁-C₆)alkyl, (C₂-C₆)alkenyl, (C₂-C₆)alkynyl, (C₃-C₈)cycloalkyl, trifluoromethyl, aryl, or aryl(C₁-C₆)alkyl, wherein each (C₁-C₆)alkyl can optionally be substituted with one or more halo, (C₁-C₆)alkanoyloxy, (C₁-C₆)alkoxy, (C₃-C₈)cycloalkyl;
each R^{m} and Rⁿ is independently H, (C₁-C₆)alkyl, (C₂-C₆)alkenyl, (C₃-C₈)cycloalkyl, (C₃-C₈)cycloalkyl(C₁-C₆)alkyl, aryl, aryl(C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
X is a linking group; and
Y is a peptide, a protein, or maleimide;
wherein rings B and C in formula I can optionally be further substituted on one or more carbons with one or more groups independently selected from the group consisting of halo, hydroxy, nitro, (C₁-C₆)alkyl, (C₁-C₆)alkenyl, (C₁-C₆)alkynyl, (C₁-C₆)alkoxy, (C₁-C₆)alkanoyl, (C₁-C₆)alkanoyloxy, (C₁-C₆)alkoxycarbonyl, trifluoromethyl, trifluoromethoxy, cyano, and NR^{p}R^{q};
each R^{p} and R^{q} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl; and
R^{r} is H, aryl, or (C₁-C₁₀)alkyl that is optionally substituted with halo or aryl;
R^{s} is H, aryl, or (C₁-C₁₀)alkyl that is optionally substituted with halo or aryl;
each R^{u} and R^{v} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{w} is H, aryl, or (C₁-C₁₀)alkyl that is optionally substituted with halo or aryl;
each R^{x} and R^{y} is independently H or (C₁-C₆)alkyl; or taken together with the nitrogen to which they are attached form a aziridino, azetidino, morpholino, piperazino, pyrrolidino or piperidino ring, which ring may optionally be substituted with one or more (C₁-C₆)alkyl;
R^{z} is H, aryl, or (C₁-C₁₀)alkyl that is optionally substituted with halo or aryl; and
R^{aa} is aryl that is optionally substituted with one or more groups independently selected from the group consisting of hydroxy, halo, nitro, cyano, (C₁-C₆)alkoxy, and (C₁-C₆)alkyl that is optionally substituted with one or more halo;
or a pharmaceutically acceptable salt thereof.

35. The compound of any one of claims 1-6, 16, 21, 23, 25-31, or 33-34, wherein R¹ is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, cyano, oxo, oxiranyl, (C₃-C₈)cycloalkyl, aryl, that is optionally substituted with alkyl that is substituted with NR^{u}R^{v}, aryl that is substituted with carboxy, heteroaryl, (C₁-C₆)alkoxy, (C₁-C₆)alkylthio, OR^{z}, —N(H)S(O)₂R^{r}, R^{s}C(═O)O—, —S—R^{w}, —NR^{x}R^{y}, (C₁-C₆)alkoxycarbonyl, and carboxy.

36. The compound of any one of claims 1-6, 16, 21, 23, 25-31, or 33-35, wherein R¹ is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, cyano, oxo, oxiranyl, (C₃-C₈)cycloalkyl.

37. The compound of any one of claims 1-6, 16, 21, 23, 25-31, or 33-36, wherein R¹ is H, (C₁-C₆)alkyl, (C₂-C₆)alkenyl, (C₂-C₆)alkynyl, or —NR^{g}R^{h}, wherein any (C₁-C₆)alkyl, (C₂-C₆)alkenyl, and (C₂-C₆)alkynyl is optionally substituted with one or more groups independently selected from the group consisting of halo, cyano, oxo, oxiranyl, (C₃-C₈)cycloalkyl.

38. The compound of any one of claims 1-6, 16, 21, 23, 25-31, or 33-37, wherein R¹ is H or (C₁-C₆)alkyl optionally substituted with one or more groups independently selected from the group consisting of halo and cyano.

39. The compound of any one of claims 1-6, 16, 21, 23, 25-31, or 33-38, wherein R¹ is H or (C₁-C₆)alkyl.

40. The compound of any one of claims 1-6, 16, 21, 23, 25-31, or 33-39, wherein R¹ is H.

41. The compound of any one of claims 1-6, 16, 21, 23, 25-31, or 33-39, wherein R¹ is not H.

42. The compound of any one of claims 1-5, 7, 9-17, 19, 21, 23, 25-31, or 33-41, wherein R² is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

43. The compound of any one of claims 1-5, 7, 9-17, 19, 21, 23, 25-31, or 33-42, wherein R² is H, (C₁-C₆)alkyl, (C₂-C₆)alkenyl, (C₂-C₆)alkynyl, (C₁-C₆)alkanoyl, (C₁-C₆)alkoxycarbonyl, or —NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, —SH, cyano, (C₃-C₈)cycloalkyl, and NR^{g}R^{h}.

44. The compound of any one of claims 1-5, 7, 9-17, 19, 21, 23, 25-31, or 33-43, wherein R² is H, (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of halo, —SH, cyano, (C₃-C₈)cycloalkyl, and NR^{g}R^{h}.

45. The compound of any one of claims 1-5, 7, 9-17, 19, 21, 23, 25-31, or 33-44, wherein R² is H, (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of halo, —SH, cyano, (C₃-C₈)cycloalkyl, and NR^{g}R^{h}.

46. The compound of any one of claims 1-5, 7, 9-17, 19, 21, 23, 25-31, or 33-45, wherein R² is H or (C₁-C₆)alkyl optionally substituted with one or more groups independently selected from the group consisting of halo, —SH, cyano, and NR^{g}R^{h}.

47. The compound of any one of claims 1-5, 7, 9-17, 19, 21, 23, 25-31, or 33-46, wherein R² is H or (C₁-C₆)alkyl.

48. The compound of any one of claims 1-5, 7, 9-17, 19, 21, 23, 25-31, or 33-47, wherein R² is H.

49. The compound of any one of claims 1-5, 7, 9-17, 19, 21, 23, 25-31, or 33-47, wherein R² is not H.

50. The compound of any one of claims 1-4, 6, 16, 25-31, or 33-49, wherein R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, or NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of hydroxy, halo, —SH, cyano, oxo, oxiranyl, (C₃-C₈)cycloalkyl, aryl, heteroaryl, (C₁-C₆)alkoxy, (C₁-C₆)alkylthio, and NR^{g}R^{h}.

51. The compound of any one of claims 1-4, 6, 16, 25-31, or 33-50, wherein R³ is H, halo, hydroxy, (C₁-C₆)alkyl, (C₂-C₆)alkenyl, (C₂-C₆)alkynyl, (C₁-C₆)alkanoyl, (C₁-C₆)alkoxycarbonyl, or NR^{g}R^{h}, wherein any (C₁-C₆)alkyl, (C₂-C₆)alkenyl, (C₂-C₆)alkynyl, (C₁-C₆)alkanoyl, and (C₁-C₆)alkoxycarbonyl is optionally substituted with one or more groups independently selected from the group consisting of halo, —SH, cyano, (C₃-C₈)cycloalkyl, and NR^{g}R^{h}.

52. The compound of any one of claims 1-4, 6, 16, 25-31, or 33-51, wherein R³ is H, (C₁-C₆)alkyl, (C₂-C₆)alkenyl, (C₂-C₆)alkynyl, or NR^{g}R^{h}, wherein any (C₁-C₆)alkyl, (C₂-C₆)alkenyl, and (C₂-C₆)alkynyl, is optionally substituted with one or more groups independently selected from the group consisting of halo, —SH, cyano, (C₃-C₈)cycloalkyl, and NR^{g}R^{h}.

53. The compound of any one of claims 1-4, 6, 16, 25-31, or 33-52, wherein R³ is H or (C₁-C₆)alkyl optionally substituted with one or more groups independently selected from the group consisting of halo, —SH, cyano, (C₃-C₈)cycloalkyl, and NR^{g}R^{h}.

54. The compound of any one of claims 1-4, 6, 16, 25-31, or 33-53, wherein R³ is H or (C₁-C₆)alkyl.

55. The compound of any one of claims 1-4, 6, 16, 25-31, or 33-54, wherein R³ is H.

56. The compound of any one of claims 1-4, 6, 16, 25-31, or 33-54, wherein R³ is not H.

57. The compound of any one of claims 13-16, 21-22, 25-31, or 33-56, wherein R⁴ is R^{k}—C(═O)—, R^{k}—O—C(═O)—, R^{k}—S(O)₂—O—, R^{c}R^{d}NS(O)₂—, R^{e}S(O)₂NR^{f}—, or 1-ethylene that is substituted at the 2-position with R^{k}—C(═O)—, R^{k}—O—C(═O)—, R^{k}—S(O)₂—O—, R^{c}R^{d}NS(O)₂—, or R^{e}S(O)₂NR^{f}—.

58. The compound of any one of claims 13-16, 21-22, 25-31, or 33-57, wherein R⁴ is R^{k}—C(═O)—, R^{k}—O—C(═O)—, or 1-ethylene that is substituted at the 2-position with R^{k}—C(═O)—, R^{k}—O—C(═O)—.

59. The compound of any one of claims 13-16, 21-22, 25-31, or 33-57, wherein R⁴ is R^{k}—S(O)₂—O—, R^{c}R^{d}NS(O)₂—, R^{e}S(O)₂NR^{f}—, or 1-ethylene that is substituted at the 2-position with R^{k}—S(O)₂—O—, R^{c}R^{d}NS(O)₂—, or R^{e}S(O)₂NR^{f}—.

60. The compound of any one of claims 13-16, 21-22, 25-31, or 33-58, wherein R⁴ is R^{k}—C(═O)— or R^{k}—O—C(═O)—.

61. The compound of any one of claims 1-19, 25-31, or 33-60, wherein the compound of Formula I comprises a structure of Formula (Ia) or Formula (Ib):

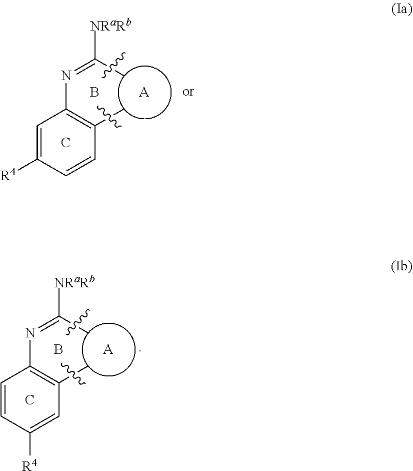

62. The compound of any one of claims 1-19, 25-31 or 33-61, wherein the compound of Formula I comprises a structure of Formula (Ia):

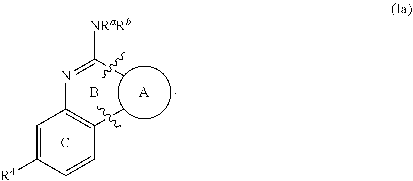

63. The compound of any one of claims 1-19, 21-23, 25-31, or 33-62, wherein R^{a} is H, (C₁-C₆)alkyl, (C₂-C₆)alkenyl, or (C₂-C₆)alkynyl, wherein any (C₁-C₆)alkyl, (C₂-C₆)alkenyl, or (C₂-C₆)alkynyl is optionally substituted with one or more groups independently selected from the group consisting of halo, —SH, cyano, oxiranyl, (C₃-C₈)cycloalkyl, (C₁-C₆)alkoxy, (C₁-C₆)alkylthio, and NR^{g}R^{h}.

64. The compound of any one of claims 1-19, 21-23, 25-31, or 33-63, wherein R^{a} is H, (C₁-C₆)alkyl, (C₂-C₆)alkenyl, or (C₂-C₆)alkynyl, wherein any (C₁-C₆)alkyl, (C₂-C₆)alkenyl, or (C₂-C₆)alkynyl is optionally substituted with one or more groups independently selected from the group consisting of halo, —SH, cyano, and NR^{g}R^{h}.

65. The compound of any one of claims 1-19, 21-23, 25-31, or 33-64, wherein R^{a} is H or (C₁-C₆)alkyl optionally substituted with one or more groups independently selected from the group consisting of halo, —SH, cyano, and NR^{g}R^{h}.

66. The compound of any one of claims 1-19, 21-23, 25-31, or 33-65, wherein R^{a} is H or (C₁-C₆)alkyl.

67. The compound of any one of claims 1-19, 21-23, 25-31, or 33-66, wherein R^{a} is H.

68. The compound of any one of claims 1-19, 21-23, 25-31, or 33-66, wherein R^{a} is not H.

69. The compound of any one of claims 1-3, 25-31, or 33-68, wherein the fused ring A is selected from the group consisting of:

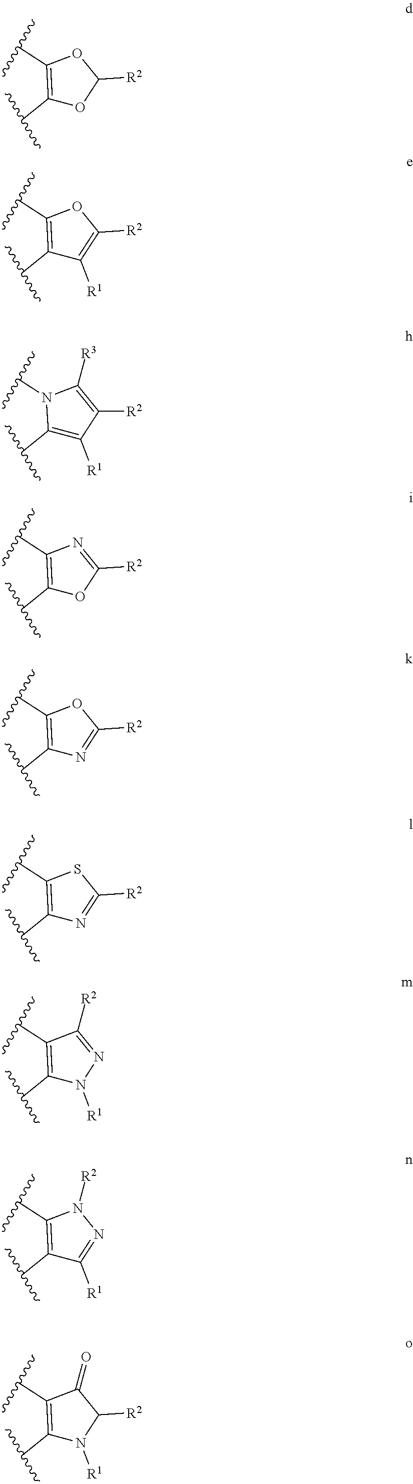

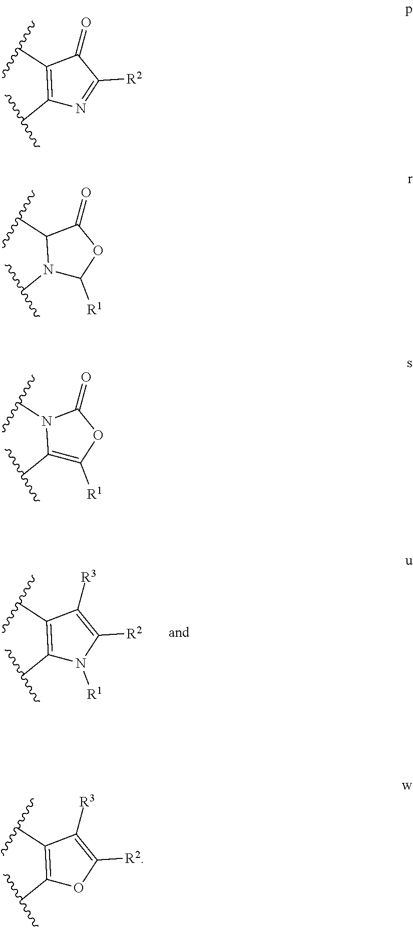

70. The compound of any one of claims 1-3, 25-31, or 33-69, wherein the fused ring A is selected from the group consisting of:

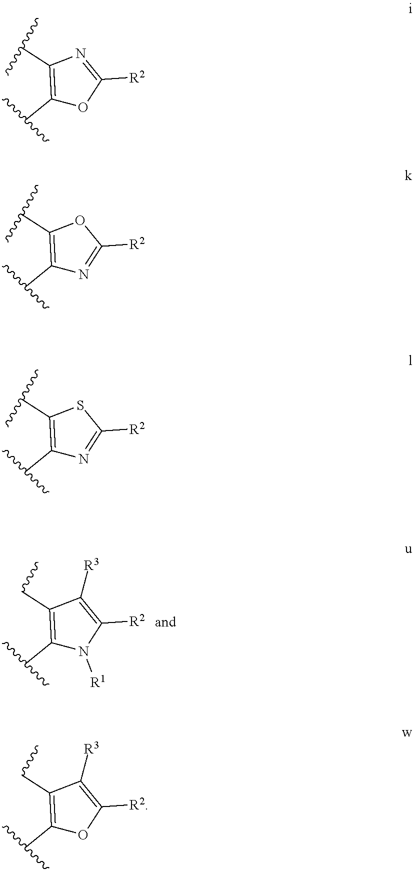

71. The compound of any one of claims 1-3, 25-31, or 33-70, wherein the fused ring A is selected from the group consisting of:

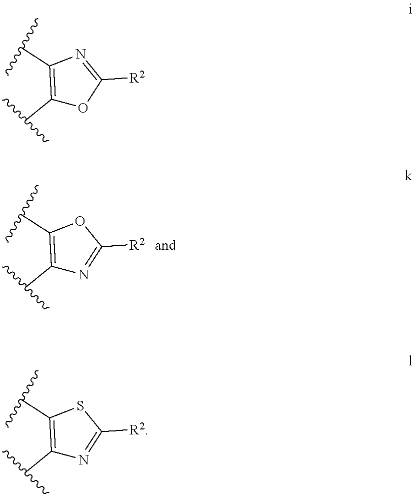

72. The compound of any one of claims 33-68, wherein fused ring A is

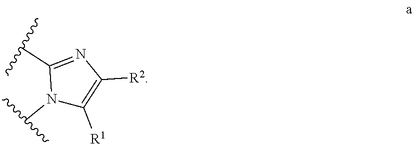

73. The compound of any one of claims 33-68, wherein fused ring A is selected from the group consisting of

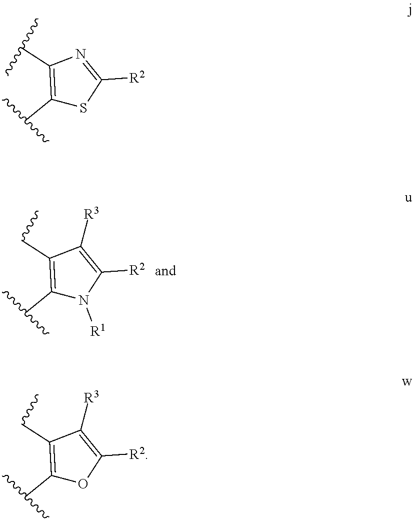

74. The compound of any one of claims 33-68 or 73, wherein fused ring A is selected from the group consisting of

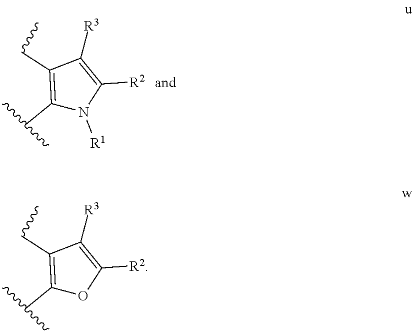

75. The compound of any one of claims 33-68 or 73, wherein fused ring A is

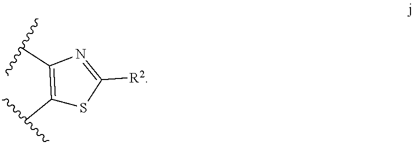

76. A pharmaceutical composition comprising a compound according to any one of claims 1-75 and a pharmaceutically acceptable diluent or carrier.

77. A method of treating a pathological condition in an animal, comprising administering to the animal a compound according to any one of claims 1-75, or a pharmaceutically acceptable salt thereof, or a pharmaceutical composition according to claim 76.

78. A method of stimulating an immune response in an animal, comprising administering to the animal a compound according to any one of claims 1-75, or a pharmaceutically acceptable salt thereof, or a pharmaceutical composition according to claim 76.

79. A method of treating a cancer in an animal, comprising administering to the animal a compound according to any one of claims 1-75, or a pharmaceutically acceptable salt thereof, or a pharmaceutical composition according to claim 76.

80. A method of synthesizing a compound of any one of claims 1-75, the method comprising performing a reaction according to any one of reaction SCHEMES 1-5.
